(19) United States
(12) Patent Application Publication
(10) Pub. No.: US 2004/0230090 A1
(43) Pub. Date: Nov. 18, 2004

(54) Vascular assist device and methods

(76) Inventors: Anant V. Hegde, Newark, CA (US); Halil I. Karabey, San Jose, CA (US)

Correspondence Address: COOLEY GODWARD, LLP, 3000 EL CAMINO REAL, 5 PALO ALTO SQUARE, PALO ALTO, CA, 94306, US

(21) Appl. No.: 10/781,357

(22) Filed: Feb. 17, 2004

Related U.S. Application Data
(63) Continuation-in-part of application No. 10/681,821, filed on Oct. 7, 2003.
(60) Provisional application No. 60/451,212, filed on Feb. 28, 2003.
(60) Provisional application No. 60/416,477, filed on Oct. 7, 2002.

(51) Int. Cl. A61N 1/362; A61M 25/00

(52) U.S. Cl. 600/18

(57) ABSTRACT

Several electroactive polymer (EAP) actuated vascular assist devices are provided that can be readily implanted within the body of a patient without coming in direct blood contact. The devices are also readily repositioned and/or removed from contact with the internal vasculature or may even be turned OFF remotely. In addition, there is provided a method of fabrication and a method of implanting such devices. There are also provided methods for the augmentation of a body lumen through the use of hemodynamic signals such as pressure or ECG signals to synchronize EAP actuation in the vascular assist system.

### CROSS-REFERENCE TO RELATED APPLICATIONS

This application claims the benefit of U.S. Provisional Application No. 60/451,212, entitled “Electroactive Polymeric Assist Device” and filed on Feb. 28, 2003, and is a continuation-in-part of U.S. patent application Ser. No. 10/681,821, entitled “Vascular Assist Device and Methods” and filed on Oct. 7, 2003, which claims the benefit of U.S. Provisional Application No. 60/416,477, entitled “Vascular Assist Device” and filed on Oct. 7, 2002, the disclosures of which are incorporated herein by reference in their entirety.

### BACKGROUND

1. Field of the Invention

The field of the present invention relates to vascular assist devices and methods, and more particularly directed to electroactive polymer vascular assist devices and conventional vascular assist devices activated by electroactive polymer pumps and actuators.

2. Description of the Related Art

Congestive heart failure is a condition that causes the heart to pump less efficiently. Typically the heart has been weakened over time by an underlying problem, such as clogged arteries, high blood pressure, a defect in its muscular walls or valves, or some other medical condition. The body depends on the heart's pumping action to deliver oxygen and nutrient-rich blood so it can function normally. In people with congestive heart failure, the body fails to get an adequate supply. As a result, they tend to feel weak, fatigued, or short of breath. Everyday activities such as walking, climbing stairs, carrying groceries and yard work can become quite difficult.

Congestive heart failure develops over time. The slow onset and progression of congestive heart failure is caused by the heart's own efforts to compensate for the weakening of the heart muscles. The heart tries to compensate for the weakening by enlarging and forcing a faster pumping rate to move more blood through the vasculature of the body.

If the left side of the heart is not working properly, blood and other fluids back up into the lungs leading to the shortness of breath and exhaustion discussed above. If the right side of the heart is not working properly, the slow blood flow causes build up of fluid in the veins causing the legs and ankles to show signs of swelling (edema). Edema often spreads to the lungs, liver, and stomach. Such a fluid buildup may also cause kidney failure due to the body's ability to dispose of salt and water. As heart failure progresses, a patient's heart becomes weaker and the symptoms begin to manifest.

People at risk for congestive heart failure may undertake various therapies to ease the workload of the heart. Such treatment may include lifestyle changes, medicines, transcatheter interventions, and surgery. While lifestyle changes and medicines are often effective non-invasive procedures that can be undertaken, they are not as effective as the alternative, albeit more invasive, procedures. That being said, transcatheter interventions and surgical procedures are highly invasive and can create substantial risk in more delicate patients (e.g., elderly people, obese people, etc.).

Examples of transcatheter interventions include angioplasty, stenting, and inotropic drug therapy. Surgical procedures include heart valve repair or replacement, pacemaker insertion, correction of congenital heart defects, coronary artery bypass surgery, mechanical assist devices, and heart transplant.

When the heart can no longer adequately function and a patient is at risk of dying it is referred to as end-stage congestive heart failure. In such cases heart transplants are often required. Mechanical assist devices such as ventricular assist devices (VADs) and axial pumps have proven to be effective in offloading the workload of the heart. These devices can act as a temporary assist for a patient's heart prior to transplant. Studies have shown that approximately twenty percent (20%) of people using VADs have recovered or healed by offloading the heart for some period of time.

Recently, ventricular assist devices have been considered as an alternative to heart transplant and have been successfully implanted in several patients worldwide. Ventricular assist devices are able to totally offload the heart, potentially leading to recovery of the heart.

There are several types of ventricular assist devices. Left ventricular assist devices that offload the left ventricle of the heart, right ventricular assist devices that offload the right ventricle of the heart and atrial assist devices that offload the atrium of the heart. These devices come into direct contact with the blood. Such direct blood contact is a major concern with respect to thrombus formation and it is necessary to give blood thinners and anticoagulants to patients fitted with such ventricular assist devices. To insert such a device it is necessary to make incisions in the heart chambers and aorta, thereby leading to infection at the implant site as well as around the conduits connecting to external devices.

Another type of assist device is the intra-aortic balloon pump (IABP). IABPs provide assistance by decreasing myocardial oxygen consumption by reducing heart afterload, as well as increasing coronary artery profusion by augmenting diastolic coronary artery flow. IABPs do not require surgical intervention to install, but rather is placed through an open approach to the common femoral artery.

Another device that is often used is an impeller, which is a miniature pump catheter that continuously pumps the blood. Aortomyplasty is another way to augment the diastolic pressure and increase coronary artery flow.

To avoid the problems of biomaterial interface and to avoid disadvantages of other known methods of increasing blood flow, devices that compress the aorta externally were developed. Such devices may often include rigid mechanical jaws that are not compliant, thereby increasing the likelihood of injury to the aorta. Additionally such devices limit the mobility of patients, thus compromising the quality of life.

Conventional vascular assist devices are often configured to increase arterial blood flow from the heart. Generally speaking, many conventional vascular assist devices are both difficult to install and cumbersome for the patient. Several vascular assist devices are configured to be inserted into the vasculature, thereby causing potential infection and other related difficulties. Other devices that are configured to be installed externally to the vasculature include many components that need to be installed in very small areas. Moreover, when the devices need to be adjusted and/or removed, complex procedures are required. Moreover, such devices also are not synchronized with the cardiac cycle, thereby not appropriately timing the compression of the aorta.

### SUMMARY OF THE INVENTION

In light of the previously described problems associated with conventional vascular assist devices, one object of the embodiments of the present invention is to provide a vascular assist device that can be readily implanted within the body of the patient without involving direct blood contact. The device is also readily repositioned and/or removed.

In one embodiment, there is provided device for engaging a body lumen including a first layer having an electroactive polymer and coupled to a second layer. The second layer having a length sufficient to at least partially encircle a body lumen and a stiffness greater than that of the first layer.

In another embodiment, there is provided a system for compressing a lumen including a cuff having an expandable layer and a cover layer. The cover layer is coupled to the expandable layer defining a cavity there between. The cavity has a volume and the cover layer defining an opening that is in fluid communication with the cavity. An electroactive polymer pump that has an output in communication with the opening, wherein the electroactive polymer pump moves a fluid to expand the expandable layer in synchronization with a portion of a cardiac cycle.

There is provided in another embodiment a device for compressing a lumen in a body comprising a cuff having a complaint layer, a semi-compliant layer coupled to the compliant layer so as to form a cavity there between; and an electroactive polymer pump in communication with the cavity.

There is provided in another embodiment a method for augmenting flow in a body lumen comprising detecting a cardiac cycle trigger; pumping a fluid through the actuation of an electroactive polymer; and deforming at least a portion of a body lumen in response to the cardiac cycle using the pumped fluid.

In yet another embodiment, there is provided a method for augmenting blood flow in a vessel comprising enlarging a cavity formed between a first layer and a second layer by activating an electroactive polymer and deforming the first layer in response to enlarging the cavity; and deforming the walls of a vessel adjacent the first layer in response to the deforming of the first layer.

In yet another embodiment there is provided a system for compressing a lumen in a body including a cuff having a compliant layer and a semi-compliant layer coupled to the compliant layer to form a cavity there between and an electroactive polymer diaphragm pump having an output. There is also a conduit connecting the output and the cavity wherein activation of the electroactive polymer diaphragm pump expands the compliant layer.

There is also provided in another embodiment a device for compressing a lumen in a body comprising a cuff having a compliant layer and a semi-compliant layer and a cavity formed between the compliant layer and the semi-compliant layer, a deformable fluid reservoir containing a fluid. There is a conduit coupling the fluid reservoir to the cavity. In addition, an electroactive polymer layer including a first electrode, a second electrode and a polymer layer disposed between the first electrode and the second electrode wherein activation of the electroactive polymer layer deforms the deformable fluid reservoir to urge the fluid into the cavity.

In another embodiment, there is a provided a system, comprising an electroactive polymer pump and a controller configured to receive a signal associated with the cardiac cycle of a heart and actuate the electroactive polymer pump in response thereto. There is also a cuff having a compliant first layer configured to engage internal vasculature; a second layer coupled to the first layer and having a stiffness greater than a stiffness of the first layer and having an opening formed therein. The compliant first layer and the second layer being coupled to form a cavity bounded by the first layer and the second layer, the cavity being in communication with the opening in the second layer. There is a conduit coupled between the opening and the electroactive polymer pump, wherein actuation of the electroactive polymer pump moves a fluid into the cavity and deforms the first layer.

In another embodiment, there is provided a system for compressing a blood vessel, comprising a cuff having an expandable layer and a cover layer, the cover layer coupled to the expandable layer defining a cavity there between; and a rolled electroactive polymer pump configured to move a fluid into the cavity to expand the expandable layer in synchronization with a portion of a cardiac cycle.

In another embodiment, there is provided a system for compressing a blood vessel, comprising a pair of lever arms coupled at a pivot point; and a rolled electroactive polymer coupled to an output shaft wherein actuation of the rolled electroactive polymer moves the output shaft; and wherein one of the lever arms is attached to the output shaft.

In yet another embodiment, there is provided a device for compressing a blood vessel, comprising a first layer comprising an electroactive polymer and coupled to a second layer; the second layer having a length sufficient to at least partially encircle a body lumen and a stiffness greater than that of the first layer; a cavity formed between the first layer and the second layer; and a bias element disposed within the cavity and configured to expand the electroactive polymer when the electroactive polymer is in an non-actuated state.

In another embodiment, there is provided a device for compressing a blood vessel in a body, comprising a deformable bladder containing a fluid; a cuff having an expandable layer and a cover layer, the cover layer coupled to the expandable layer to define a cavity there between; and a “C” ring electroactive polymer actuator disposed about the bladder such that actuation of the electroactive polymer actuator deforms the bladder and forces fluid into the cavity.

In another embodiment, there is provided a method for augmenting blood flow in a body, comprising sensing the R wave of the ECG of the body; computing the QT interval to the end of the T wave; and actuating an electroactive polymer based vascular assist system in relation to the T wave.

In another embodiment, there is provided a method for augmenting blood flow in a body by sensing a pressure wave related to a hemodynamic pressure in the body; and based on a portion of the pressure wave, actuating an electroactive polymer based system to augment blood flow in the body.

In yet another embodiment, there is provided a method of forming a stacked electroactive polymer actuator by forming a plurality of adjacent electrodes on a single polymer layer; and folding the polymer layer so that adjacent electrodes are stacked so that at least a single polymer layer exists between each adjacent electrode.

Another object of the embodiments of the present invention is to provide a method of fabrication and a method of implanting such a vascular assist device.

A further object of the embodiments of the present invention is to provide a method including increasing a pressure of a liquid or gas in an aortic cuff based on a control signal related to the systole and/or diastole of the heart and/or the aortic pressure.

Other objects, advantages and features associated with the embodiments of the present invention will become more readily apparent to those skilled in the art from the following detailed description. As will be realized, the invention is capable of other and different embodiments and its several details are capable of modification in various obvious aspects, all without departing from the invention. Accordingly, the drawings and the description are regarded as illustrative in nature, and not limiting.

### BRIEF DESCRIPTION OF THE DRAWINGS

Embodiments of the present invention are described with reference to the accompanying drawings. In the drawings, like reference numbers indicate similar elements.

FIG. 1 includes Table A entitled “Comparison of Electroactive Polymer (EAP) Types.”

FIG. 2 includes Table B entitled “EAP Material Requirement.”

FIGS. 3A and 3B are perspective views of an inactivated (FIG. 3A) and actuated (FIG. 3B) dielectric electroactive polymer actuator.

FIG. 4 is a perspective view of an exemplary ion-exchange polymer metal composite electroactive polymer actuator.

FIGS. 5A and 5B illustrate an exemplary diaphragm pump in an inactivated state (FIG. 5A) and actuated state (FIG. 5B).

FIGS. 6A and 6B illustrate a perspective view (FIG. 6A) and an exploded view (FIG. 6B) of an embodiment of a stacked multi-layered electroactive polymer actuator of the present invention.

FIGS. 7A, 7B, 7C, and 7D illustrate alternative electrode shape embodiments for multi-layer electroactive polymer actuators of the present invention.

FIGS. 8A, 8B, 8C, 8D, and 8E illustrate various views of an illustrative rolled electroactive polymer actuator.

FIGS. 9A , 9B, and 9C illustrate various views of a multi-stage rolled electroactive polymer actuator.

FIGS. 10A and 10B illustrate cross section views of electroactive polymer actuator assemblies.

FIG. 11 is a perspective view of a single polymer layer used for a stacked electrode actuator.

FIG. 12 is illustrates an embodiment of an electroactive polymer pump actuated vascular assist system of the present invention.

FIGS. 13A and 13B illustrate section views A-A of the electroactive polymer pump embodiment of FIG. 12 in actuated (FIG. 13B) and inactivated (FIG. 13A) modes.

FIGS. 14A, 14B, and 14C illustrate perspective, exploded and section views of an exemplary expandable cuff vascular assist device.

FIG. 15 is a section view of an alternative electroactive polymer actuated pump according to one embodiment of the present invention.

FIGS. 16A, 16B, 16C, and 16D illustrate several views of a single chamber electroactive polymer actuated diaphragm pump according to one embodiment of the present invention.

FIGS. 16E and 16F illustrate EAP actuators having positive (FIG. 16E) and negative (FIG. 16F) bias.

FIGS. 17A, 17B, 17C, and 17D illustrate several views of a single chamber electroactive polymer actuated diaphragm pump according to another embodiment of the present invention.

FIGS. 18A, 18B, 18C, and 18D illustrate several views of a dual chamber electroactive polymer actuated diaphragm pump according to an embodiment of the present invention.

FIGS. 19A, 19B, 19C, and 19D illustrate several views of two embodiments of an electroactive polymer actuated vascular assist system of the present invention.

FIG. 20 is a system view of an embodiment of an electroactive polymer actuated vascular assist system of the present invention implanted in a human body.

FIG. 21 is a section view of an embodiment of a multi-chamber EAP pump with a single input.

FIG. 22 illustrates a cross section view of an embodiment of a multi-chamber EAP pump having an inlet and an outlet.

FIG. 23 is a perspective view of an embodiment of a planar cross-connected multi-chamber EAP.

FIGS. 24A and 24B are views of an embodiment of a multi-chamber array EAP pump.

FIG. 25 is a schematic view of an embodiment of an EAP actuated vascular augmentation system having an embodiment of an EAP cuff.

FIGS. 26A, 26B, 27A and 27B are cross section views of alternative embodiments of the EAP cuff of FIG. 25.

FIGS. 28A and 28B illustrate various views of an embodiment of a minimally invasive EAP actuated cuff.

FIGS. 29, 30, and 31 illustrate several views of an embodiment of an EAP cuff.

FIGS. 32A and 32B illustrate alternative embodiments of vascular assist EAP devices of the present invention.

FIG. 33 illustrates an embodiment of a vascular assist EAP cuff of the present invention in position to augment blood flow in the ascending aorta.

FIGS. 34A and 34B are EAP cuffs having fabric for securing the cuff about a vessel.

FIG. 35 is a perspective view of an EAP cuff having an embodiment of a vessel protection layer of the present invention.

FIGS. 36A and 36B illustrate embodiments of a segmented EAP actuated cuff of the present invention.

FIGS. 37A and 37B illustrate segmented cuffs according to embodiments of the present invention.

FIGS. 38A, 38B, 38C, 38D, 39A, 39B, 40A, 40B, 40C, 40D, 41A, 41B, 42, 43, 44, 45A, 45B, 46, and 47 illustrate various alternative embodiments of connection mechanisms for coupling cuffs of the present invention about body lumens.

FIGS. 48A, 48B, and 48C illustrate an embodiment of a rolled EAP with radial actuation.

FIGS. 49A and 49B illustrate an embodiment of a rolled EAP with axial actuation.

FIGS. 50A, 50B, and 50C are rolled EAP actuators on a vessel compression device.

FIG. 51 is an embodiment of a diaphragm actuation coupled to a shaft.

FIG. 52 is an embodiment of a plurality of rolled EAP actuators on a body lumen.

FIG. 53 is an illustrative embodiment of a multiple rolled EAP actuators on a vessel compression device.

FIG. 54 is another embodiment of a rolled EAP actuator driving another vessel compression device.

FIG. 54 is another embodiment of a rolled EAP actuator on a vessel compression device.

FIGS. 55A and 55B schematically illustrate an energy efficient operating scheme for high-energy utilization.

FIG. 56 illustrates a high efficiency EAP pump used to drive a piston and actuate fluid for actuation of inflatable cuffs of the present invention.

FIG. 57 contains “Comparison of Assist Device Technologies” (Table C).

FIG. 58 is a conventional screw driven vascular assist system.

FIG. 59 is a conventional impeller driven vascular assist system

FIG. 60 is a conventional total artificial heart (TAH).

FIG. 61 illustrates representative pressure and ECG waves generated by an embodiment of the vascular assist system of the present invention operated in copulsation mode.

FIG. 62 illustrates representative pressure and ECG waves generated by an embodiment of the vascular assist system of the present invention operated in counterpulsation mode.

### DETAILED DESCRIPTION

The following documents discuss electroactive polymer actuator materials, fabrication techniques and device application. Each document listed below is incorporated by reference in its entirely for all purposes.

1. Pelrine et al., “Electroactive Polymer Electrodes,” U.S. Pat. No. 6,376,971, issued Apr. 23, 2002.

2. Pelrine et al., “Electroactive Polymer Electrodes,” U.S. patent application Ser. No. 09/993,871, filed on Nov. 15, 2001, allowed, to be issued.

3. Pelrine et al., “Electroactive Polymer Fabrication,” U.S. Pat. No. 6,543,110, issued Apr. 8, 2003.

4. Pelrine et al., “Electroactive Polymer Transducers and Actuators,” U.S. patent application Ser. No. 09/620,025, filed on Jul. 20, 2000.

5. Pelrine et al., “Electroactive Polymer Devices,” U.S. Pat. No. 6,545,384, issued Apr. 8, 2003.

6. Pelrine et al., “Improved Electroactive Polymers,” U.S. patent application Ser. No. 09/619,847, filed on Jul. 20, 2000.

7. Pelrine et al., “Monolithic Electroactive Polymers,” U.S. patent application Ser. No. 09/779,203, filed on Feb. 7, 2001.

8. Pelrine et al., “Energy Efficient Electroactive Polymers and Electroactive Polymers Devices,” U.S. patent application Ser. No. 09/779,373, filed on Feb. 7, 2001.

9. Pelrine et al., “Electroactive Polymer Sensors,” U.S. patent application Ser. No. 10/007,705, filed on Dec. 6, 2001.

10. Pelrine et al., “Electroactive Polymer Devices for Moving Fluid,” U.S. patent application Ser. No. 10/393,506, filed on Mar. 18, 2003.

11. Heim et al., “Electroactive Polymer Devices for Controlling Fluid Flow,” U.S. patent application Ser. No. 10/383,005, filed on Mar. 5, 2003.

12. Pei et al., “Rolled Electroactive Polymers,” U.S. patent application Ser. No. 10/154,449, filed on May 21, 2002.

13. Pelrine et al., “Electroactive Polymers,” European Patent Application No. EP2000000959149, filed on Jul. 20, 2000.

14. Pelrine et al., “Electroactive Polymers,” Japanese Patent Application No. 2001-510928, filed on Jul. 20, 2000.

15. Pelrine et al., “Improved Electroactive Polymers,” European Patent Application No. EP2000000948873, filed on Jul. 20, 2000.

16. Pelrine et al., “Improved Electroactive Polymers,” Japanese Patent Application No. 2001-510924, filed on Jul. 20, 2000.

17. Heim et al., “Electroactive Polymer Devices for Controlling Fluid Flow,” PCT Patent Application No. US03/07115, filed on Mar. 5, 2003.

18. Pelrine et al., “Electroactive Polymer Devices for Moving Fluid,” PCT Patent Application (number not yet assigned), filed on Mar. 18, 2003.

19. Shahinpoor, et al., “Soft Actuators and Artificial Muscles,” U.S. Pat. No. 6,109,852 issued Aug. 29, 2000.

20. Shahinpoor, et al., “Ionic Polymer Sensors and Actuators,” U.S. Pat. No. 6,475,639 issued Nov. 5, 2002.

Electroactive Polymers Types and Characteristics:

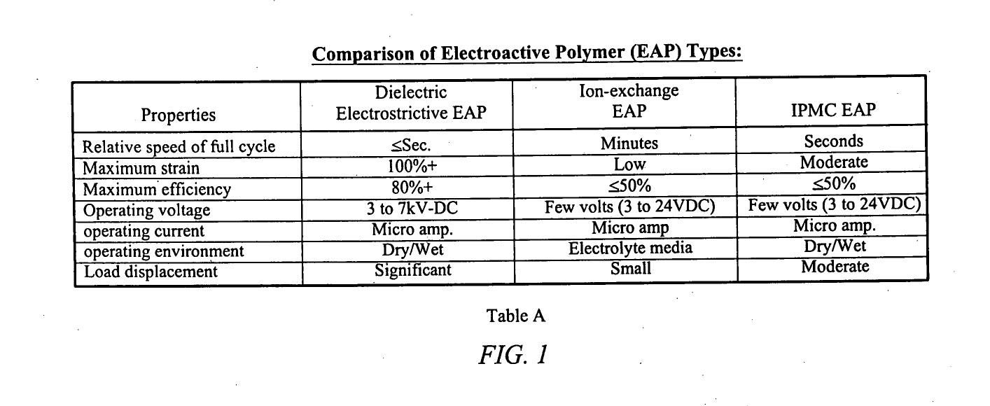

FIG. 1 includes Table A that is entitled “Comparison of Electroactive Polymer (EAP) Types” and compares several properties of electroactive polymers (EAP) namely, dielectric electostrictive electroactive polymers, ion-exchange electroactive polymers and ionomeric polymer-metal composite (IPMC) electroactive polymers. For most vascular assist applications, the relative speed of full cycle or response time of the material is an important design consideration. Given that the resting human heart beats anywhere from about 50 to 80 beats per minute, existing dielectric electostrictive EAP and IPMC EAP provide a response time within a useful range for vascular assist embodiments of the present invention. Still more responsive EAPs are under development and those materials may also be advantageously employed in embodiments of the present invention. On the other hand, the current state of ion-exchange EAP materials have not yet reached the same desirous performance characteristics of the dielectric electostrictive electroactive polymers, and ion-exchange electroactive polymers. However, advancements in ion-exchange EAP are underway and more responsive ion-exchange materials, when developed, can also be used in the vascular augmentation embodiments of the present invention. In view of the forgoing, it is to be appreciated that the term electroactive polymer as used herein refers generally to the above described and other types of materials that repeatably deflect when exposed to an actuation source.

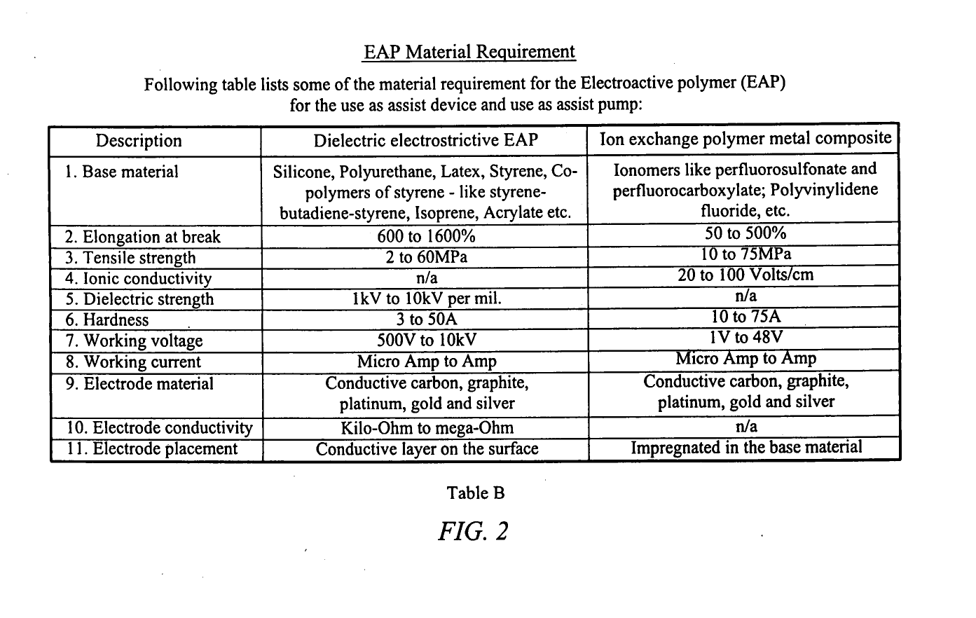

FIG. 2 includes a Table B that is entitled “EAP Material Requirement” that includes some of the desired material characteristics of two of the existing EAP materials suited to the vascular augmentation embodiments of the present invention. Table B details some of the material requirements for electroactive polymer materials that may be advantageously employed in the vascular assist devices, assist pumps and system embodiments of the present invention. The material details provided in Tables A and B are for purposes of illustration and not limitation. Other materials under development will provide even more response and efficient EAPs suited to the novel vascular assist applications described herein. Numerous publications exist that detail more completely the state of the art in EAP development. One of the more comprehensive discussions of all areas of EAP development is “Electroactive Polymer (EAP) Actuators as Artificial Muscles: Reality, Potential and Challenges” by Yoseph Bar Cohen (Editor) (2001). This book is incorporated by reference in its entirely for all purposes. The above listed and incorporated patents and patent applications to Pelrine et al., Heim et al., Pei et al. and Shahinpoor further describe the current state of the art of electroactive polymer actuators, devices and systems.

The present invention is described in detail with reference to a few preferred embodiments as illustrated in the accompanying drawings. In the following description, numerous specific details are set forth in order to provide a thorough understanding of the present invention. It will be apparent, however, to one skilled in the art, that the present invention may be practiced without some or all of these specific details. In other instances, well known process steps and/or structures have not been described in detail in order to not unnecessarily obscure the present invention.

Brief Discussion of Electroactive Polymers:

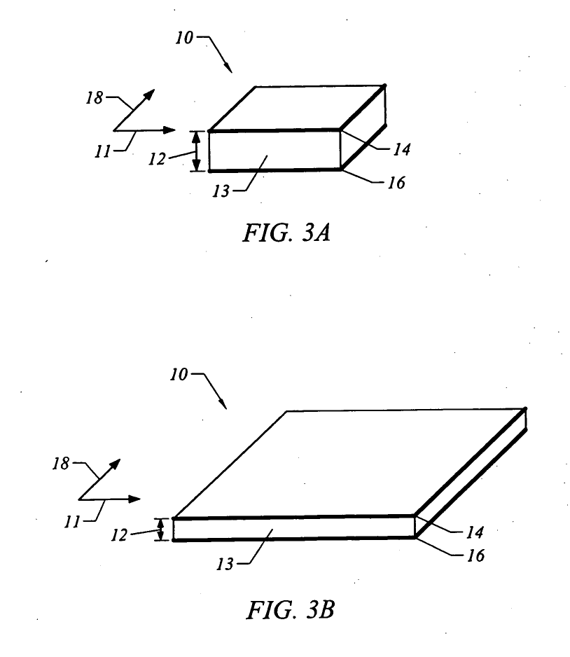

Before describing electroactive polymer vascular assist devices of embodiments of the present invention, the basic principles of electroactive polymer construction and operation will first be described with reference to FIG. 3A and FIG. 3B. Embodiments of EAP cuffs, pumps, devices, and systems of the present invention are described in greater detail below. The transformation between electrical and mechanical energy in devices of the present invention is based on energy conversion of one or more active areas of an electroactive polymer. Electroactive polymers are capable of converting between mechanical energy and electrical energy. In some cases, an electroactive polymer may change electrical properties (for example, capacitance and resistance) with changing mechanical strain.

To help illustrate the performance of an electroactive polymer in converting between electrical energy and mechanical energy, FIG. 3A illustrates a top perspective view of an exemplary electroactive polymer actuator 10. The electroactive polymer actuator 10 comprises an elastomeric polymer layer 13 between a pair of compliant electrodes 14 and 16 configured for converting between electrical energy and mechanical energy. The elastomeric polymer layer 13 refers to a polymer that acts as an insulating dielectric between two electrodes and may deflect upon application of a voltage difference between the two electrodes 14 and 16 (a ‘dielectric elastomer’). Top and bottom electrodes 14 and 16 are attached to the polymer 13 on its top and bottom surfaces, respectively, to provide a voltage difference across polymer 13, or to receive electrical energy from the polymer 13. Polymer 13 may deflect with a change in electric field provided by the top and bottom electrodes 14 and 16. Deflection of the electroactive polymer 10 in response to the application of an appropriate actuation energy, here in response to a change in electric field provided by the electrodes 14 and 16, is referred to as ‘actuation’. Actuation typically involves the conversion of electrical energy to mechanical energy. The deflection of polymer 13 as it changes size may then be used to produce mechanical work.

Without wishing to be bound by any particular theory, in some embodiments, the polymer 13 may be considered to behave in an electostrictive manner. The term electostrictive is used here in a generic sense to describe the stress and strain response of a material to the square of an electric field. The term is often reserved to refer to the strain response of a material in an electric field that arises from field induced intra-molecular forces but we are using the term more generally to refer to other mechanisms that may result in a response to the square of the field. Electrostriction is distinguished from piezoelectric behavior in that the response is proportional to the square of the electric field, rather than proportional to the field. The electrostriction of a polymer with compliant electrodes may result from electrostatic forces generated between free charges on the electrodes (sometimes referred to as “Maxwell stress”) and is proportional to the square of the electric field. The actual strain response in this case may be quite complicated depending on the internal and external forces on the polymer, but the electrostatic pressure and stresses are proportional to the square of the field.

FIG. 3B illustrates a top perspective view of the electroactive polymer actuator 10 in an actuated condition and including deflection. In general, deflection refers to any displacement, expansion, contraction, torsion, linear or area strain, or any other deformation of a portion of the polymer 13. For actuation, a change in electric field corresponding to the voltage difference applied to or by the electrodes 14 and 16 produces mechanical pressure within polymer 13. In this case, the unlike electrical charges produced by electrodes 14 and 16 attract each other and provide a compressive force between electrodes 14 and 16 and an expansion force on polymer 13 in planar directions 18 and 11, causing polymer 13 to compress between electrodes 14 and 16 and stretch in the planar directions 18 and 11.

As is well known, electrodes 14 and 16 are compliant and change shape with polymer 13. The configuration of polymer 13 and electrodes 14 and 16 provides for increasing polymer 13 response with deflection. More specifically, as the electroactive polymer 10 deflects, compression of polymer 13 brings the opposite charges of electrodes 14 and 16 closer and the stretching of polymer 13 separates similar charges in each electrode. In some embodiments, one of the electrodes 14 and 16 is ground. During actuation of the electroactive polymer actuator 10, the polymer layer 13 continues to deflect until mechanical forces balance the electrostatic forces driving the deflection. The mechanical forces include elastic restoring forces of the polymer 13 material, the compliance of electrodes 14 and 16, and any external resistance provided by a device, load or bias member coupled to the electroactive polymer actuator 10. The deflection of the electroactive polymer actuator 10 as a result of an applied voltage may also depend on a number of other factors such as the polymer 13 dielectric constant and the size of polymer 13.

Electroactive polymers in accordance with embodiments of the present invention are capable of deflection in any direction. After application of a voltage between the electrodes 14 and 16, the electroactive polymer 13 increases in size in both planar directions 18 and 11. In some cases, the electroactive polymer 13 is incompressible, e.g. has a substantially constant volume under stress. In this case, the polymer 13 decreases in thickness as a result of the expansion in the planar directions 18 and 11. It should be noted that the present invention is not limited to incompressible polymers and deflection of the polymer 13 may not conform to such a simple relationship.

Application of a relatively large voltage difference between electrodes 14 and 16 on the electroactive polymer actuator 10 shown in FIG. 3A will cause the polymer layer 13 to change to a thinner, larger area shape as shown in FIG. 3B. In this manner, the electroactive polymer actuator 10 converts electrical energy to mechanical energy. The electroactive polymer actuator 10 may also be used to convert mechanical energy to electrical energy.

Turning now to a brief discussion of the composition and general operation of ion-exchange polymer metal composite electroactive polymers. Ion-exchange polymer metal composite electroactive polymers are actuators that incorporate the use of ion-exchange membrane actuators made from ion-exchange membranes (or any ionomer membrane, ion-exchange resin, gel, beads, powder, filaments, or fiber) by chemically, mechanically and electrically treating them with at least one noble metal such as platinum. Ion-exchange polymer metal composite electroactive polymers are described more fully in “Soft Actuators and Artificial Muscles,” U.S. Pat. No. 6,109,852 issued Aug. 29, 2000 to Shahinpoor, et al., and “Ionic Polymer Sensors and Actuators,” U.S. Pat. No. 6,475,639, issued Nov. 5, 2002 to Shahinpoor, et al. Ion-exchange membranes (or any ionomer membrane) such as a perflourinated sulfonic acid polymer or an ionomer such as Nafion®, available from DuPont Corporation, Fayetteville, N.C. Nafion® is a perfluorinated sulfonic acid ion-exchange polymer membrane having industrial applications for separation processes, production of caustic sodas and fuel cell applications.

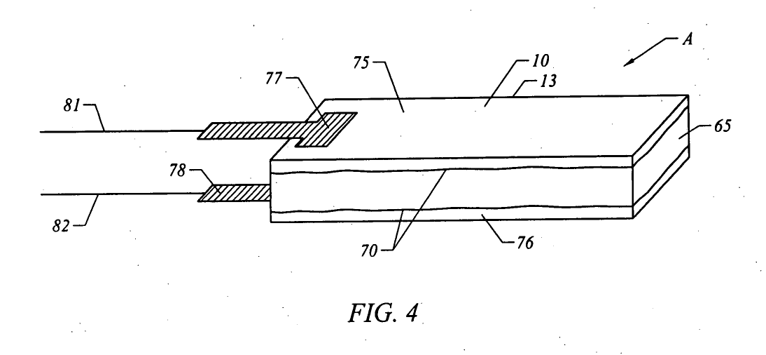

FIG. 4 depicts such an exemplary ion-exchange polymer metal composite electroactive polymer actuator made by chemically and mechanically treating Nafion®. membranes with platinum. FIG. 4 is a perspective view of a treated planar membrane actuator A. The treated Nafion® membrane 65 is sandwiched between compliant electrodes 75, 76. Compliant electrodes 75, 76 are connected to power supply 85 via terminal connections 77, 78 and wires 81, 82. When actuated, the membrane 65, along with the compliant electrodes 75, 76, deflect. This deflection is adjustable and controllable and may be used to produce useful work.

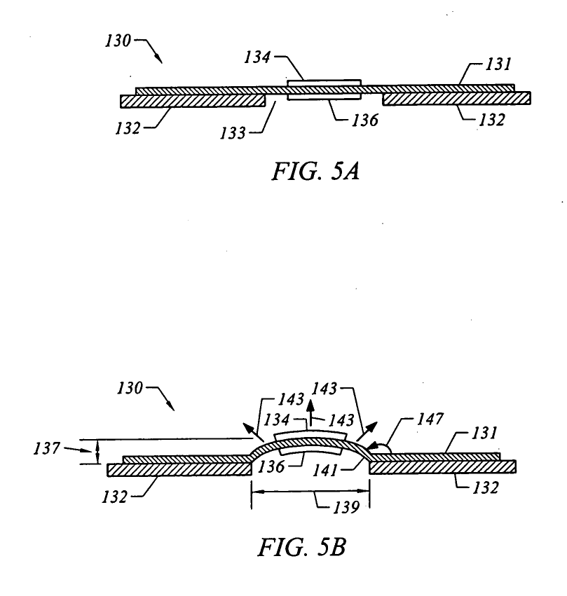

Operation of EAP actuators may be better appreciated through reference to the actuation of a simple diaphragm pump. A diaphragm pump 130 is illustrated in an inactivated state (FIG. 5A) and an actuated state (FIG. 5B). FIG. 5A illustrates a cross-sectional side view of a diaphragm actuator 130 including a polymer 131 in an inactivated state. The polymer 131 may be pre-strained before being attached to a frame 132. The frame 132 includes a circular hole 133 that allows deflection of the polymer 131 perpendicular to the area of the circular hole 133. The diaphragm actuator 130 includes circular electrodes 134 and 136 on either side of the polymer 131 to provide a voltage difference across a portion of the polymer 131.

In the inactivated or voltage-off configuration of FIG. 5A, the polymer 131 is stretched and secured to the frame 132 with tension to achieve pre-strain, if desired. Upon application of a suitable voltage to the electrodes 134 and 136, the polymer film 131 expands away from the plane of the frame 132 as illustrated in FIG. 5B. The electrodes 134 and 136 are compliant and change shape with the polymer 131 as it deflects.

The amount of expansion for the diaphragm actuator 130 will vary based on a number of factors including the polymer 131 material, the applied voltage, the amount of pre-strain, any bias pressure, compliance of the electrodes 134 and 136, etc. In some embodiments, the polymer 131 is capable of deflections to a height 137 of at least about 50 percent of the diameter 139 and may take a hemispheric shape at large deflections. In this case, an angle 147 formed between the polymer 131 and the frame 132 may be less than 90 degrees.

Electroactive polymer actuators used in the present invention are not limited to any particular actuator type, shape, rolled geometry or type of deflection. For example, the polymer and electrodes may be formed into any geometry or shape including tubes and multi-layer rolls, rolled polymers attached between multiple rigid structures, rolled polymers attached across a frame of any geometry—including curved or complex geometries, across a frame having one or more joints, etc. Similar structures may be used with polymers in flat sheets. Deflection of an actuator as used herein includes linear expansion and compression in one or more directions, bending, axial deflection when the polymer is rolled, deflection out of a hole provided on an outer cylindrical around the polymer, etc. Deflection of an actuator may be affected by how the polymer is constrained by a frame or rigid structures attached to the polymer.

Exemplary materials suitable for use as an electroactive polymer include any substantially insulating polymer or rubber (or combination thereof) that deforms in response to an electrostatic force or whose deformation results in a change in electric field. One suitable material is Nosily CF19-2186 as provided by Nosily Technology of Carpentaria, Calif. Other exemplary materials suitable for use as a polymer include any dielectric elastomeric polymer, silicone rubbers, silicone elastomers, acrylic elastomers such as VHB 4910 acrylic elastomer as produced by 3M Corporation of St. Paul, Minn., silicones such as Dow Coming HS3 as provided by Dow Coming of Wilmington, Del., fluorosilicones such as Dow Coming 730 as provided by Dow Coming of Wilmington, Del., etc, and acrylic polymers such as any acrylic in the 4900 VHB acrylic series as provided by 3M Corp. of St. Paul, Minn., polyurethanes, thermoplastic elastomers, copolymers comprising PVDF, pressure-sensitive adhesives, fluoroelastomers, polymers comprising silicone and acrylic moieties, and the like. Polymers comprising silicone and acrylic moieties may include copolymers comprising silicone and acrylic moieties, polymer blends comprising a silicone elastomer and an acrylic elastomer, for example. Combinations of some of these materials may also be used as the electroactive polymer in actuators employed by embodiments of the vascular assist devices of the present invention.

Materials to be used as an electroactive polymer may be selected based on one or more material properties such as a high electrical breakdown strength, a low modulus of elasticity—(for large or small deformations), a high dielectric constant, etc. In one embodiment, the polymer is selected such that is has an elastic modulus at most about 100 MPa. In another embodiment, the polymer is selected such that is has a maximum actuation pressure between about 0.05 MPa and about 10 MPa, and preferably between about 0.3 MPa and about 3 MPa. In another embodiment, the polymer is selected such that is has a dielectric constant between about 2 and about 20, and preferably between about 2.5 and about 12. For some applications, an electroactive polymer is selected based on one or more application demands such as a wide temperature and/or humidity range, repeatability, accuracy, low creep, reliability and endurance.

An electroactive polymer layer in actuators used in embodiments of the present invention may have a wide range of thicknesses. For example, polymer thickness may range between about 1 micrometer and 2 millimeters. Polymer thickness may be reduced by stretching the film in one or both planar directions. In many cases, electroactive polymers of the present invention may be fabricated and implemented as thin films. Thicknesses suitable for these thin films may be below 50 micrometers.

As electroactive polymers of the present invention may deflect at high strains, electrodes attached to the polymers should also deflect without compromising mechanical or electrical performance. The ability of the electrodes to deflect and conform with the polymer layer during actuation is generally referred to as compliance. Suitable electrodes may be of any shape and material provided that they are able to supply a suitable voltage to, or receive a suitable voltage from, a polymer layer. The voltage may be either constant or varying over time. In some electroactive polymer actuators, the electrodes adhere to a surface of the polymer. Electrodes adhering to the polymer are preferably highly compliant and conform to the changing shape of the polymer during actuation. As such, electroactive polymer actuators used herein may include compliant electrodes that conform to the shape of an electroactive polymer to which they are attached. The electrodes may be only applied to a portion of an electroactive polymer and define an active area according to their geometry. Several examples of electrodes that only cover a portion of an electroactive polymer will be described in further detail below.

Various types of electrodes suitable for use with electroactive polymer actuators used by the novel vascular augmentation devices and systems of the present invention are described in co-pending U.S. patent application Ser. No. 09/619,848, which was previously incorporated by reference above. Electrodes described therein and suitable for use include structured electrodes comprising metal traces and charge distribution layers, textured electrodes comprising varying out of plane dimensions, conductive greases such as carbon greases or silver greases, colloidal suspensions, high aspect ratio conductive materials such as carbon fibrnls and carbon nanotubes, and mixtures of ionically conductive materials.

Materials used for electrodes may vary. Suitable materials used in an electrode may include graphite, carbon black, colloidal suspensions, thin metals including silver and gold, silver filled and carbon filled gels and polymers, and ionically or electronically conductive polymers. Other suitable electrode material include conductive carbon, graphite, platinum, gold and silver.

It is understood that certain electrode materials may work well with particular polymers and may not work as well for others. By way of example, carbon fibrils work well with acrylic elastomer polymers while not as well with silicone polymers. For most actuators, desirable properties for the compliant electrode may include one or more of the following: low modulus of elasticity, low mechanical damping, low surface resistivity, uniform resistivity, chemical and environmental stability, chemical compatibility with the electroactive polymer, good adherence to the electroactive polymer, and the ability to form smooth surfaces. In some cases, an electroactive polymer may include two different types of electrodes, e.g. a different electrode type for each active area or different electrode types on opposing sides of a polymer.

In some cases, the electrodes cover a limited portion of the polymer relative to the total area of the polymer. This may done to prevent electrical breakdown around the edge of polymer or achieve customized deflections in certain portions of the polymer. As the term is used herein, an active region is defined as a portion of the polymer material having sufficient electrostatic force to enable deflection of the portion. As will be described below, electroactive polymers may advantageously utilize multiple active regions. Polymer material outside an active area may act as an external spring force on the active area during deflection. More specifically, material outside the active area may resist active area deflection by its contraction or expansion. Removal of the voltage difference and the induced charge causes the reverse effects.

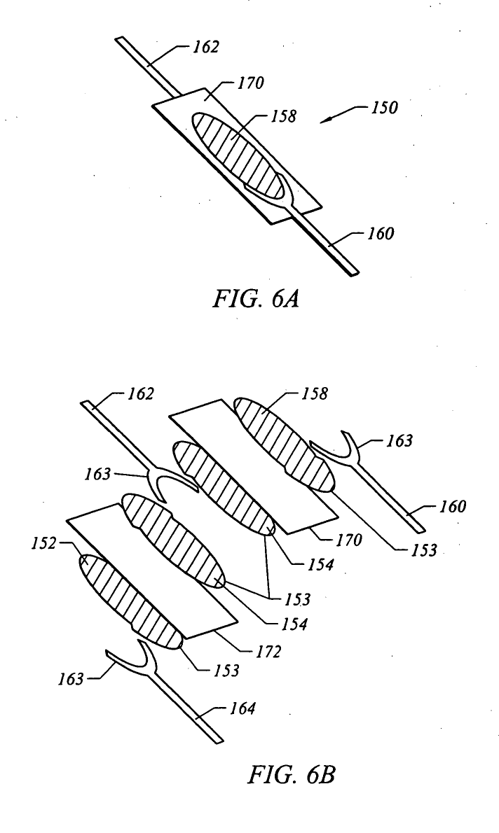

FIG. 6A and FIG. 6B illustrate a perspective and exploded view of an embodiment of a multi-layer electroactive polymer actuator 150 of the present invention. The stacked multi-layer electroactive polymer actuator 150 includes compliant electrodes 152, 154, 156, 158 that change shape with the deflection of polymer layers 172, 170. Conductors 164 and 160 couple actuation energy, here electric power from a power source, (not shown) to the electrodes 152 and 158, respectively at attachment point 153. Advantageously, conductor 162 couples actuation energy, here electric power from a power source, (not shown) to the electrodes 154 and 156. For example, conductors 164, 160 may be connected to a positive electrical potential making electrodes 158 and 152 cathodes while conductor 162 may be connected to a negative electrical potential making electrodes 154, 156 anodes. The electrical potential attached to the conductors may also be changed. The number of polymer/electrode stacks is not limited to that illustrated in this embodiment. Additional polymer layers and electrodes may be added. In that case, conductors 160 and 164 may be used to power two electrodes as in the illustrated embodiment where conductor 162 powers both electrodes 154, 156. The configuration of the polymer layers and electrodes provides for increasing polymer layer response with deflection.

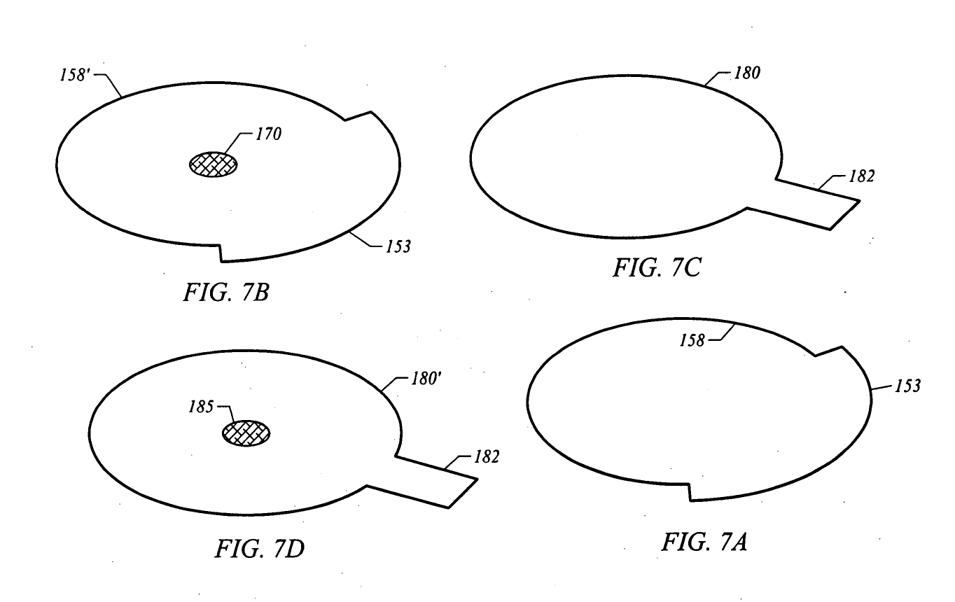

The electrodes 152, 154, 156 and 158 have a single shaped end 153 with a flared, accurate portion to provide a readily identifiable attachment point for the conductors. This design provides for similar manufacturing processes (described below) as well as increased electrical and mechanical reliability. Note that each electrode advantageously has only one shaped end 153 for conductor attachment. By having only one attachment point the electrodes may be stacked as shown in FIG. 6B with reduced likelihood that an electrical short may occur. The conductors for negative potential attach to electrodes on one side and conductors for positive potential attaching to electrodes on the other side (i.e., conductors 160 and 164 at one potential and conductor 162 at the other potential). FIG. 7A-7D illustrate alternative electrode shape embodiments for multi-layer electroactive polymer actuators of the present invention. FIG. 7A illustrates an electrode 158 with an accurate attachment point 153 that is similar to the electrodes illustrated in FIG. 6B above. FIG. 7B illustrates another electrode embodiment that is electrode 158′. Electrode 158′ has an accurate attachment point 153 and includes an inactive portion 170. Inactive portion 170 is a non-conductive area of the electrode 158′. The inactive portion 170 provides an attachment point for a bias element (not shown), such as a metal spring, to be attached and provide bias force to the electroactive polymer actuator while reducing the risk that electrical malfunction will occur by having a conductive bias element adjacent an electrode. Electrodes 180 and 180′ provide alternative electrode shapes having a rectangular single attachment point 182 (FIG. 7C and FIG. 7D). FIG. 7D illustrates an inactive region 185 in the electrode 180′. Inactive regions 185, 170 are provided for illustration and not limitation. The inactive region may be in other shapes instead of the illustrated circular shape and the shape may be similar to or different than the overall shape of the electrode. The size of the inactive region may be a larger percentage of the electrode surface than is illustrated and may also change depending on the type of bias element used.

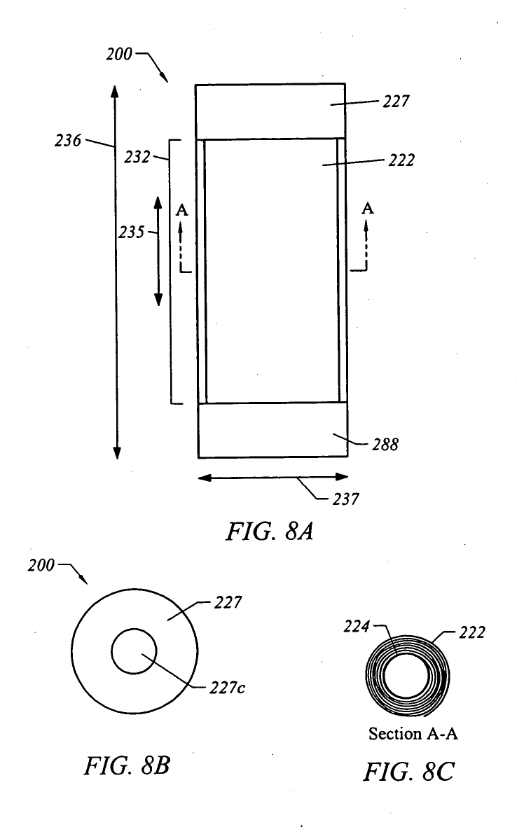

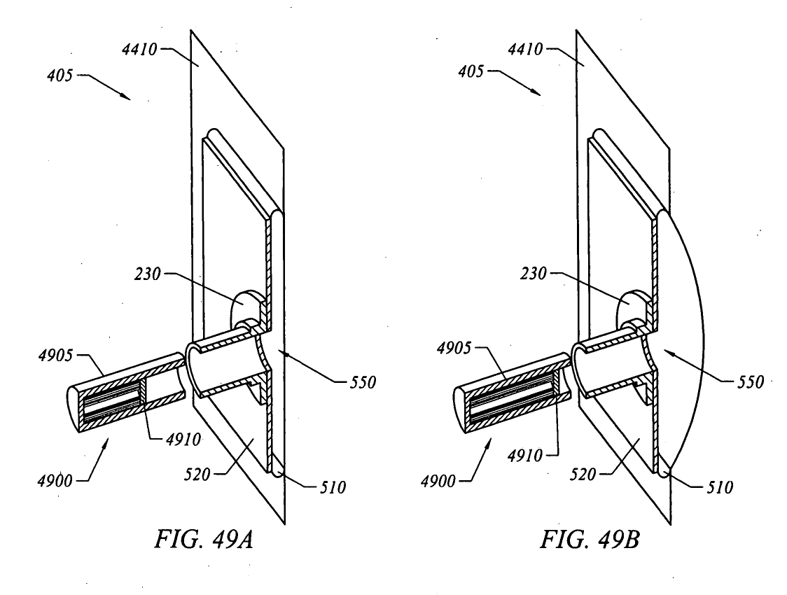

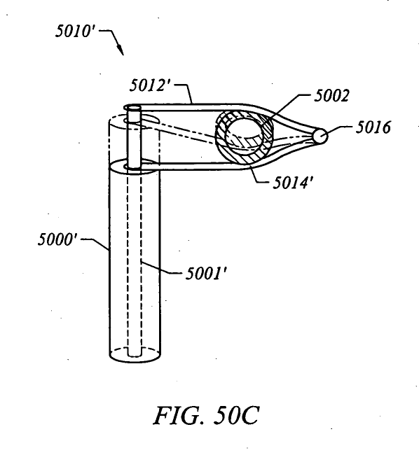

FIGS. 8A-8D illustrate an exemplary embodiment of a rolled electroactive polymer device 200 that may be used in embodiments of the augmentation devices and systems of the present invention. Embodiments of the rolled electroactive polymer device illustrated may be used for actuation of an embodiment of a lumen compression device (e.g., see FIGS. 50A, B and C, 52, 53 and 54) and may also act as part of a fluid conduit (e.g., see FIGS. 48A-C, 49A,B). In addition, rolled electroactive polymer devices may provide linear and/or rotational/torsional motion for vascular augmentation. FIG. 8A illustrates a side view of device 200. FIG. 8B illustrates an axial view of device 200 from the top end. FIG. 8C illustrates an axial view of device 200 taken through cross section A-A of FIG. 8A. FIG. 8D illustrates components of device 200 before rolling. Rolled electroactive polymer actuator 200 comprises a rolled electroactive polymer 222, spring 224, end pieces 227 and 228, electrode connections 242, 241 to provide actuation energy (e.g., electric potential) to the active regions (not shown) of the electroactive polymer 222 and various fabrication components used to hold device 200 together.

As illustrated in FIG. 8C, electroactive polymer 222 is rolled. In one embodiment, a rolled electroactive polymer refers to an electroactive polymer with, or without electrodes, wrapped round and round onto itself (e.g., like a poster) or wrapped around another object or a bias element such as a torsion spring 224. The polymer may be wound repeatedly and at the very least comprises an outer layer portion of the polymer overlapping at least an inner layer portion of the polymer. In one embodiment, a rolled electroactive polymer refers to a spirally wound electroactive polymer wrapped around an object or center. As the term is used herein, rolled is independent of how the polymer achieves its rolled configuration.

1181 As illustrated by FIGS. 8C and 8D, electroactive polymer 222 is rolled around the outside of spring 224. Electrode power connectors 242, 241 are provided to supply actuation energy to electrodes (not shown) to actuate the polymer 222. A plurality of electrodes may be arranged about the polymer 222 as described below in FIG. 8E. Additionally, more than one connector may be provided and individually controlled. Spring 224 provides a bias force that strains at least a portion of polymer 222. The top end 224a of spring 224 is attached to rigid end piece 227. Likewise, the bottom end 224b of spring 224 is attached to rigid end piece 228. The top edge 222a of polymer 222 (FIG. 8D) is wound about end piece 227 and attached thereto using a suitable adhesive. The bottom edge 222b of polymer 222 is wound about end piece 228 and attached thereto using an adhesive. Thus, the top end 224a of spring 224 is operably coupled to the top edge 222a of polymer 222 in that deflection of top end 224a corresponds to deflection of the top edge 222a of polymer 222. Likewise, the bottom end 224b of spring 224 is operably coupled to the bottom edge 222b of polymer 222 and deflection bottom end 224b corresponds to deflection of the bottom edge 222b of polymer 222. Polymer 222 and spring 224 are capable of deflection between their respective bottom top portions.

As is well known, many electroactive polymers perform better when prestrained. For example, some polymers exhibit a higher breakdown electric field strength, electrically actuated strain, and energy density when prestrained. Spring 224 of device 200 provides forces that result in both circumferential and axial prestrain onto polymer 222.

Spring 224 is a compression spring that provides an outward force in opposing axial directions (FIG. 8A) that axially stretches polymer 222 and strains polymer 222 in an axial direction. Thus, spring 224 holds polymer 222 in tension in axial direction 235. In one embodiment, polymer 222 has an axial prestrain in direction 235 from about 50 to about 300 percent. As is described further in the above incorporated patents and patent applications, device 200 may be fabricated by rolling a prestrained electroactive polymer film around spring 224 while it the spring is compressed. Once released, spring 224 holds the polymer 222 in tensile strain to achieve axial prestrain.

Spring 224 also maintains circumferential prestrain on polymer 222. The prestrain may be established in polymer 222 longitudinally in direction 233 (FIG. 8D) before the polymer is rolled about spring 224. Techniques to establish prestrain in this direction during fabrication are described in the above incorporated patents and patent applications. Fixing or securing the polymer after rolling, along with the substantially constant outer dimensions for spring 224, maintains the circumferential prestrain about spring 224. In one embodiment, polymer 222 has a circumferential prestrain from about 100 to about 500 percent. In many cases, spring 224 provides forces that result in anisotropic prestrain on polymer 222.

The application of actuation energy to the polymer layer 222 may be accomplished in a number of ways. For example, an electrode may be attached to each side of the polymer and run the entire length. While such an actuation scheme holds the promise of simplicity, there may be advantages to driving the polymer 222 through the use of a plurality of electrodes spread across the polymer surface. As used herein, an active area exists where an electrode is attached to the polymer. In some rolled electroactive polymer actuators, a plurality of active areas may exist on a single polymer and may be individually actuated or actuated in concert. FIG. 8E illustrates an exemplary multiple active area electroactive polymer actuator 260 having a plurality of active areas on a single polymer 262. The multiple active area electroactive polymer actuator 260 comprises an electroactive polymer 262 having two active areas 262a and 262b. Polymer 262 may be held in place using, for example, a rigid frame (not shown) attached at the edges of the polymer.

Active area 262a has top and bottom electrodes 264 and 266 that are attached, respectively, to the top and bottom surfaces of the polymer 262. Active area 262b has top and bottom electrodes 268 and 270 that are attached, respectively, to the top and bottom surfaces of the polymer 262. Electrodes 264 and 266 provide or receive electrical energy across a portion 262a of polymer 262. Portion 262a may deflect with a change in electric field provided by the electrodes 264 and 266. For actuation, portion 262a comprises the polymer 262 between the electrodes 264 and 266 and any other portions of the polymer 262 having sufficient electrostatic force to enable deflection upon application of voltages using the electrodes 264 and 266. When active area 262a is used as a generator to convert from electrical energy to mechanical energy, deflection of the portion 262a causes a change in electric field in the portion 262a that is received as a change in voltage difference by the electrodes 264 and 266.

Active area 262b has top and bottom electrodes 268 and 270 that are attached, respectively, to the top and bottom surfaces of the polymer 262. Electrodes 268 and 270 provide or receive electrical energy across a portion 262b of polymer 262. Portion 262b may deflect with a change in electric field provided by the electrodes 268 and 270. For actuation, portion 262b comprises the polymer 262 between the electrodes 268 and 270 and any other portions of the polymer 262 having sufficient electrostatic force to enable deflection upon application of voltages using the electrodes 268 and 270. When active area 262b is used as a generator to convert from electrical energy to mechanical energy, deflection of the portion 262b causes a change in electric field in the portion 262b that is received as a change in voltage difference by the electrodes 268 and 270. Wires (not shown) connect the electrodes to a power source and control system for actuation of the active areas simultaneously, sequentially or serially to achieve the desired actuation of the rolled electroactive polymer actuator.

Active areas for an electroactive polymer may be easily patterned and configured using conventional electroactive polymer electrode fabrication techniques. Multiple active area polymers and transducers are further described in U.S. Pat. No. 6,664,718, which is incorporated herein by reference for all purposes. Given the ability to pattern and independently control multiple active areas allows rolled transducers described herein to be utilized advantageously in embodiments of the vascular augmentation devices and systems of the present invention described below.

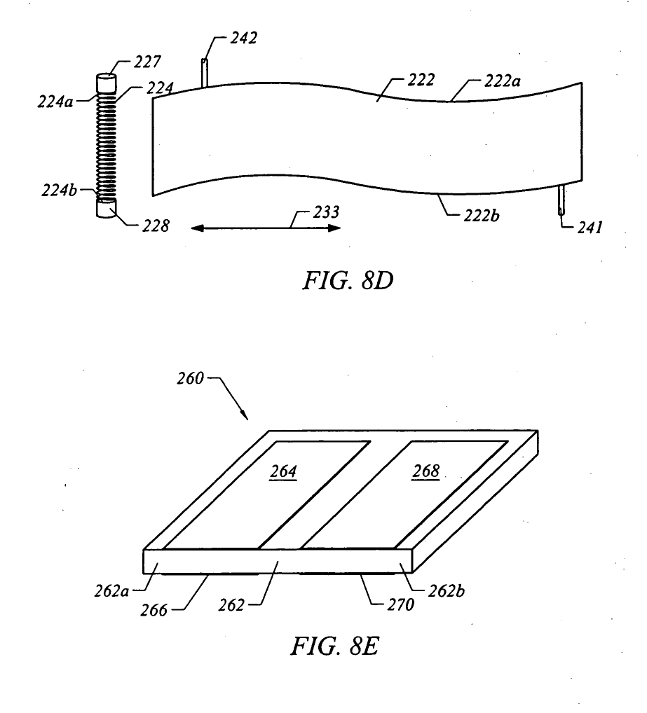

Rolled electroactive polymer actuators may also be configured to have an increased stroke (FIGS. 9A-9C). In one illustrative configuration, a nested arrangement is used to increase the stroke of a rolled electroactive polymer actuator. In a nested arrangement, one or more electroactive polymer rolls are placed in the hollow central part of another electroactive polymer roll.

FIGS. 9A-9C illustrate exemplary cross-sectional views of a nested electroactive polymer device 300, taken through the vertical midpoint of the cylindrical roll, in accordance with one embodiment of the present invention. Nested device 300 comprises three electroactive polymer rolls 302, 304, and 306. Each polymer roll 302, 304, and 306 includes a single active area that provides uniform deflection for each roll. Electrodes for each polymer roll 302, 304, and 306 may be electrically coupled to actuate (or produce electrical energy) in unison, or may be separately wired for independent control and performance. The bottom of electroactive polymer roll 302 is connected to the top of the next outer electroactive polymer roll, namely roll 304, using a connector 305. Connector 305 transfers forces and deflection from one polymer roll to another. Connector 305 preferably does not restrict motion between the rolls and may comprise a low friction and insulating material, such as Teflon. Likewise, the bottom of electroactive polymer roll 304 is connected to the top of the outermost electroactive polymer roll 306. The top of polymer roll 302 is connected to an output shaft 308 that runs through the center of device 300. Although nested device 300 is shown with three concentric electroactive polymer rolls, it is understood that a nested device may comprise another number of electroactive polymer rolls.

Output shaft 308 may provide mechanical output for device 300 (or mechanical interface to external objects). Bearings may be disposed in a bottom housing 312 and allow substantially frictionless linear motion of shaft 308 axially through the center of device 300. Housing 312 is also attached to the bottom of roll 306 and includes bearings that allow travel of shaft 308 through housing 312.

The deflection of shaft 308 comprises a cumulative deflection of each electroactive polymer roll included in nested device 300. More specifically, individual deflections of polymer roll 302, 304 and 306 will sum to provide the total linear motion output of shaft 308. FIG. 9A illustrates nested electroactive polymer device 300 with zero deflection. In this case, each polymer roll 302, 304 and 306 is in an inactivated (rest) position and device 300 is completely contracted. FIG. 9B illustrates nested electroactive polymer device 300 with 20% strain for each polymer roll 302, 304 and 306. Thus, shaft 308 comprises a 60% overall strain relative to the individual length of each roll. Similarly, FIG. 9C illustrates nested electroactive polymer device 300 with 50% strain for each polymer roll 302, 304 and 306. In this case, shaft 308 comprises a 150% overall strain relative to the individual length of each roll. By nesting multiple electroactive polymer rolls inside each other, the strains of individual rolls add up and provide a larger net stroke than would be achieved using a single roll. Nested electroactive polymer rolled devices are then useful for applications requiring large strains and compact packages, such as embodiments of the augmentation devices and systems of the present invention.

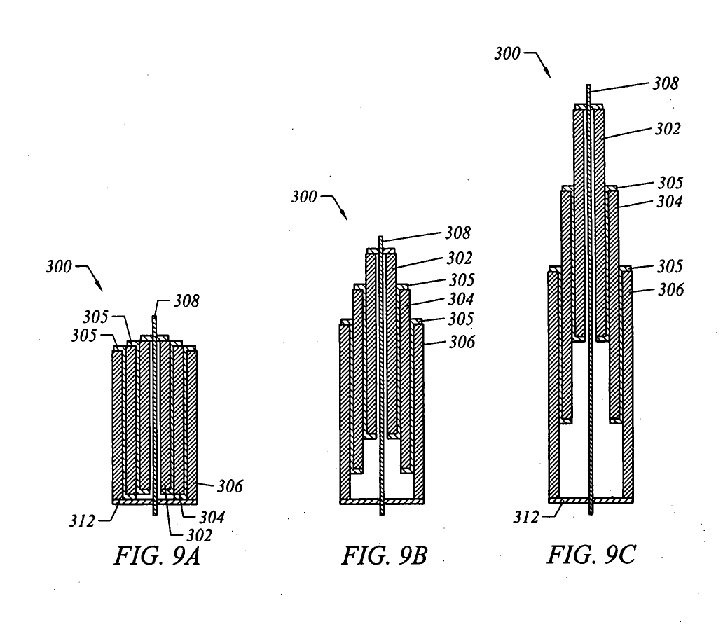

FIGS. 10A and 10B illustrate enlarged cross section views of electroactive polymer actuators. FIG. 10A illustrates a conventional electroactive polymer 350 having a dielectric polymer layer 356 between electrodes 352 and 354. Polymer layer 356 includes a pocket, void, inconsistent micro property or defect 358 that has been enlarged for purposes of illustration and discussion. As electroactive polymeric actuator 350 repeats numerous actuation cycles, the likelihood that defect 358 will become larger and potentially become an open electrical pathway between the electrodes 352 and 354 increases. If defect 358 were to become so large as to create an open electrical pathway between the electrodes 352 and 354 the electroactive polymer actuator 350 would fail to operate. This scenario is one example how actuator reliability is adversely impacted by a non-uniformity in the material either inherent or induced during a manufacturing process. One technique to remedy the problem illustrated in FIG. 10A is to obtain polymer layer material of such high manufacturing quality that defects, such as defect 358, exist in the polymer layer to such a low degree that the likelihood that the defect would create an electrical short is low. However, the costs associated with such a high-quality manufacturing processes would likely result in actuators that are not economically feasible to manufacture. Another disadvantage of the conventional electroactive polymer actuator 350 configuration is that because there is only a single polymer layer 356 between the electrodes any failure of that layer will result in a failure of the actuator 350.

In view of these shortcomings of conventional electroactive polymer actuators, an improved electroactive polymer actuator 360 will now be described with reference to FIG. 10B. Unlike electroactive polymer 350, electrodes 352 and 354 in electroactive polymer 360 are separated by a plurality of polymer layers (362, 364 and 366) rather than only a single polymer layer (356). Polymer layers 362, 364, and 366 are thinner than the single polymer layer 356 but when stacked have the same overall thickness as actuator 350. Polymer layers 362, 364, and 366 also have defects 358. However, because of the randomness of the defects 358 within the polymer layers it is unlikely that defects will appear in adjacent layers in a continuous line to result in an electrical breakdown that traverses each layers in the combined polymer layer stack. The use of the multi-polymer layer approach described herein will improve the dielectric properties and mechanical tear resistance of EAP actuators that advantageously employ this technique. In this manner, the use of lower quality polymer layers having including defects is mitigated by using a plurality of polymer layers, where the failure of any one layer will not necessarily lead to the overall failure of the actuator. Because the advantageous multi-polymer layer design of actuator 360 mitigates the risk posed by polymer layer defects, less expensive, lower commercial grade (i.e., lower quality) polymer layers may be used. As a result, the fabrication of electroactive polymer actuators 360 is possible at lower cost, and with easier manufacturability. While the advantages of a multi-polymer layer actuator design has been described with regard to actuator 360 in FIG. 10B, is to be appreciated that the principles described above and advantages and increased actuator reliability of the multi-polymer layer design may be applied to other actuator designs described herein.

In some embodiments of the electroactive polymer actuators of the present invention the EAP actuator has an anode surface, a cathode surface and an elastomer material separating the anode surface from the cathode surface. In alternative embodiments, an insulating layer is disposed adjacent the anode surface such that the anode surface is between the insulating layer and an elastomer material. In still other alternative embodiments there is an insulating layer disposed adjacent the cathode surface such that the cathode surface is between the insulating layer and an elastomer material.

In some embodiments of the present invention where the EAP is actuated using electrodes the anode and cathode conductivity is about 750 ohms to 1 mega-ohm. In some embodiments, the polymer material in the EAP is an elastomer material that separates the anode surface from the cathode surface and has a dielectric strength is about 1 kV to 10 kV per mil. In another embodiment, the elastomer material separating the anode surface from the cathode surface hardness is about 3 A to 75 A durometer. In still another embodiment, the elastomer material separating the anode surface from the cathode surface tensile strength is about 2 to 75 MPa.

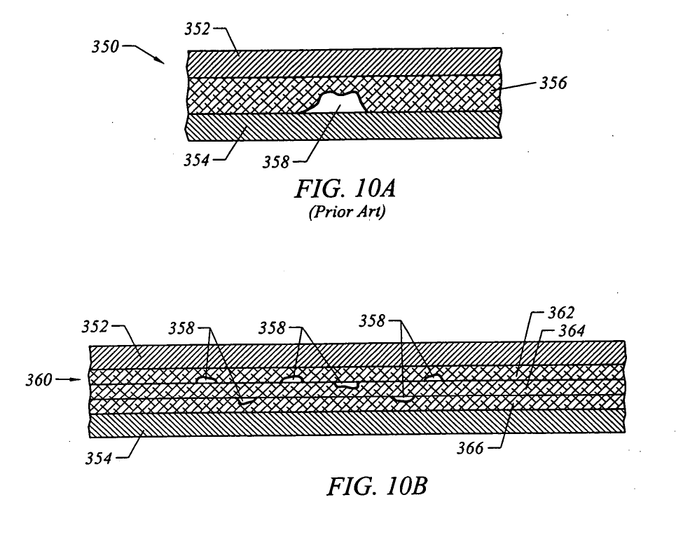

FIG. 11 illustrates a perspective view of an embodiment of a single polymer layer stack electrode electroactive polymer actuator 370. First, a plurality of electrodes, 372, 374 and power connection points 376 are fabricated on a single polymer layer 371. That the each electrode advantageously has only a single power connection point 376 (i.e., see FIG. 6A, 6B above and electrode stack 150). The electrodes may be formed using inexpensive, commercial deposition techniques, such as a silk screening, printing, spraying and the like. The electrodes are formed with sufficient spacing alone. The polymer layer 371 may then be folded along a plurality of creases 378. The polymer layer 371 is folded along creases 378, as indicated by the arrows, resulting in folded portions of the polymer layer 371 being sandwiched between an electrode 378 and an electrode 372. Once polymer layer 371 has been folded, the resulting multi-electrode polymer layer stack may be sealed using an adhesive or other conventional techniques. Advantageously, the electrical power connection points 376 for electrodes 372 are aligned together on the same side, and, at the same time, power connection points 376 for electrodes 378 are also present on the same side. By advantageously using only a single connection point for each electrode the resulting stack of electrodes at the same potential (i.e., anodes or cathodes) can be driven from a single power connection point 376 because once folded along the creases, the power connection points 376 align in a vertical stack.

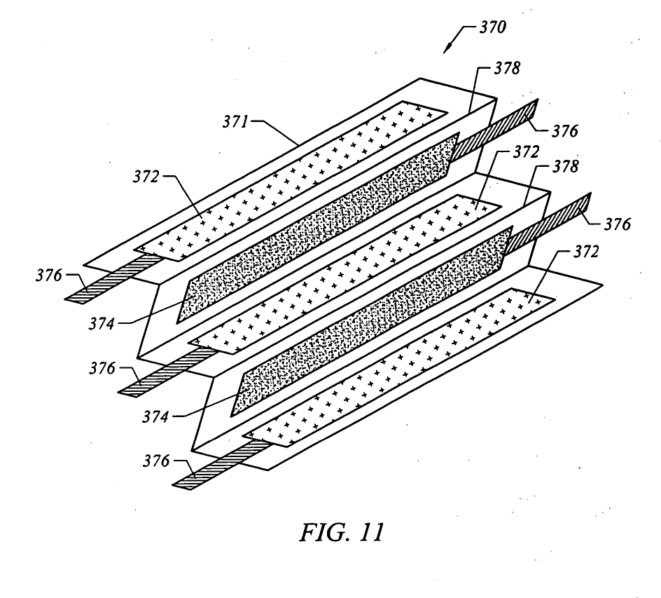

FIG. 12 illustrates an electroactive polymer actuated vascular assist system 400 according to one embodiment of the present invention. In some embodiments, each of the vascular assist system 400 components is implantable within a body. The vascular assist system 400 includes a vascular assist device 405 coupled to a pump 410 via a conduit 415. The vascular assist device 405 is a fluid inflatable cuff having a cover layer coupled to an expandable layer. A cavity is defined by the cover layer and the expandable layer. The vascular assist device 405 is configured to encircle and come into contact with the outer wall of a body lumen 402.

One advantage of some of the embodiments of the vascular assist devices of the present invention is that the devices do not come into contact with the body blood supply (i.e., the vascular assist devices remain outside the vasculature being augmented). In addition, devices and systems of the invention may be turned out without risk of harming the person whose vasculature is being assisted. In most cases, the devices and systems according to embodiments of the invention will fail in a mode that releases a vessel or assume an unaugmented position about the body lumen.

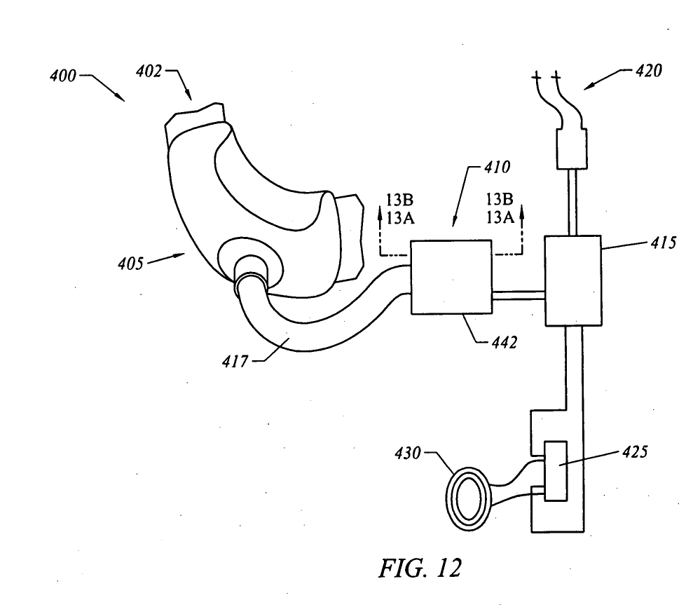

The pump 410 is an electroactive polymer actuated pump. FIGS. 13A and 13B illustrate a section view (A-A of FIG. 12) of the pump 410. A conduit 415 (i.e., a hollow flexible tube) connects the pump 410 to the cuff 405. A bladder 435 is disposed within or operably in relation to the electroactive polymer actuators 440 and 445 within a pump casing 442. The bladder 435 is a flexible non-compliant, semi-compliant or deformable chamber that stores the fluid 417 used to operate vascular assist device 405 (i.e., fill the cavity with fluid 417 to expand the expandable layer and compress a body lumen 402). In operation, actuated of the electroactive polymer actuators 440, 445 manipulates the bladder 435 resulting in fluid 417 movement.

FIG. 13A illustrates the pump, 410 prior to actuation of the electroactive polymer actuators 440, 445. Numerous details of the actuators 440, 445, such as, for example, electrical connections, electrode and polymer layer of positions have been omitted for clarity. When actuation energy is applied to the electroactive polymer actuators 440, 445, the actuators 440, 445 deform and compress the bladder 435. When bladder 435 is compressed, fluid 417 is forced out of the bladder 435 as indicated by arrow 443. The actuators 440, 445 are then unpowered and the elastic forces of the cuff 405 force fluid 417 back into bladder 435 in the direction indicated by arrow 444 (FIG. 13A). The elastic return force of cuff 405 may be the only force used to expand bladder 435 and actuators 440, 445 or the elastic cuff force may be combined with other biasing or return force elements coupled to actuators 440, 445 or bladder 435.

Operation of the pump 410 (i.e., activation and de-activation of actuators 440 and 445) for the actuation of the vascular assist device 405 is controlled by the pacing and pump controller 415. The pacing and pump controller 415 includes a programmable computer and electronics for operating the components of vascular assist system 400. Sensors 420, such as, for example, pressure sensors or electronic sensors, are positioned to detect, in one embodiment, a signal representing the cardiac cycle of a heart in a patient body. A signal representing the cardiac cycle of a heart in a patient body may be, for example, an electrical signal related to the cardiac rhythm, or the blood pressure, such as, in a blood vessel, for example, the aorta or the vena cava or pressure measured elsewhere on the patient body to indicate arterial or venous blood pressure. A battery 425 provides power to the components of the vascular assist system 400. In the illustrated embodiment, internal coils 430 are also provided so that the battery may be charged transcutaneously.

In operation, the pacing and pump control 415 may, for example, interpret the signal representing the cardiac cycle detected by the sensors 420, execute control signals to pump 410 based on the cardiac rhythm to port fluid into or out of the vascular assist device 405, record cardiac activity, or execute pre-programmed routines for the actuation of the vascular assist device 405. For example, to cause compression of a body lumen 402, the pacing and pump controller 415 signals the pump 410 to actuate electroactive polymer actuators 440, 445 and compress the bladder 435. Compression of bladder 435 forces the fluid 417 into the cuff 405 resulting in the inflation of the cuff 405. As will be described in greater detail below, the cuff 405 is positioned in relation to a body lumen, a blood vessel for example, such that cuff 405 inflation results in compression of the body lumen. As will be described below, cuff activation and body lumen compression can be advantageously synchronized with a number of parameters that are related to the cardiac cycle of a heart in a patient body.

A variety of different type of sensors 420 may be used in vascular assist system 400 for monitoring the cardiac cycle of a heart. In one embodiment, the sensor 420 may be a pressure sensor. One suitable pressure sensor may be, for example, a pressure gage that is coupled (i.e., either integrally coupled or removably coupled) directly to the cuff 405. Alternatively, the pressure of the blood in a vessel may be measured with a pressure catheter positioned internally within the vessel. In yet another alternative, the sensor 420 may be a pressure transducer suited for measuring blood pressure within a vessel or any portion of the patient body where blood pressure may be detected and used by the system 400. A suitable pressure transducer may be either internal to or externally disposed about or within the vessel of interest. In an alternative embodiment, the sensor 420 may be an electrical sensor suited for detecting an electrical signal associated with the cardiac cycle of the heart. In some embodiments, the electrical sensor is an electrocardiogram (ECG) lead. It is to be appreciated that some embodiments of the cuff 405 comprise embodiments of the pressure sensor and/or the electrical sensor. The embodiments of the pressure sensor and/or electrical sensor may be disposed directly adjacent the cuff 405 or integrally formed in the cuff 405.

As will be described further below, an embodiment of the sensor 420 may be used to detect a signal related to the cardiac cycle of a heart. The signal is then used by the pacing and pump controller, in some embodiments, as the trigger for the activation of the cuff 405. In one embodiment, the sensor 420 is a pressure sensor and the signal related to the cardiac cycle of the heart is the pressure in a vessel. The vessel measured may also depend on the location of the cuff 405 and the desired augmentation scheme. For example, if arterial augmentation is desired, the cuff 405 will likely be implanted on the arterial side of the heart about the aorta. In this example, the pressure sensor would be disposed to measure aortic pressure. On the other hand, if venous augmentation is desired, the cuff 405 will likely be implanted on the venous side of the heart about the vena cava. In this example, the pressure sensor may be disposed to measure venous pressure in the vena cava (i.e., in either the inferior or superior vena cava) or use a measurement of arterial side pressure.

The fluid 417 used within the vascular assist system 400 may be any of a wide variety of biocompatible fluids. The fluid 417 may be a liquid, such as, for example, saline, water, a glycol, such as for example, ethylene glycol. In addition the liquid may also be a mixture comprising water and a glycol or a mixture comprising saline and a glycol. The system fluid may also be a gas such as a gas that is chemically inert with the materials used to form the components in communication with the fluid. Components in communication with the fluid 417 include, for example, the cuff 405 and the conduit 415. For example, when the cuff 405 is formed from a material such as of silicone, neoprene and copolymers comprising styrene and butadiene then examples of inert gases include carbon dioxide or nitrogen. Alternatively, the system fluid may also be a gas having a density less than air. As used herein, a density less than air refers to a density less than either 1.2928 grams/liter or 0.08071 lb./cu. ft. at a standard temperature and pressure (STP) of 0 degrees C. and 760 mm Hg. Examples of suitable gases having a density less than air are helium (density of 0.1785 grams/liter or 0.01143 lb./cu. ft.); and nitrogen (density of 1.2506 grams/liter or 0.078072 lb./cu. ft.).

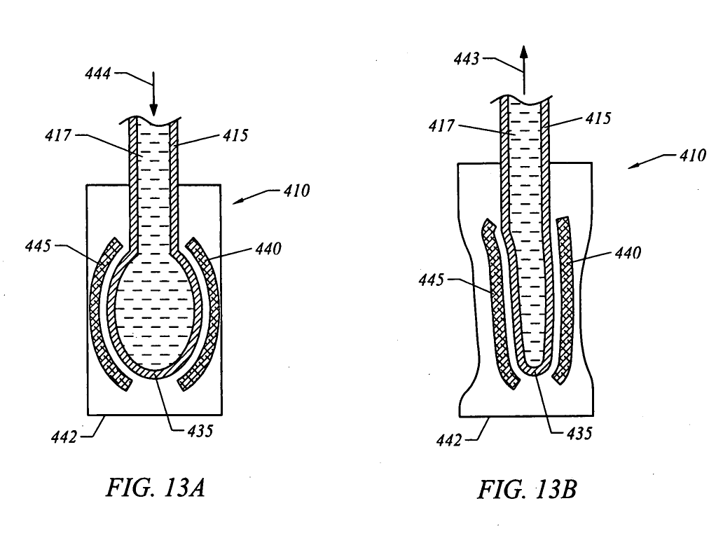

FIGS. 14A, 14B and 14C illustrate an embodiment of an inflatable cuff that may be actuated using an electroactive polymer pump embodiment according to the present invention. The ventricular assist device or inflatable cuff 405 includes a compliant first layer or expandable wall 510 that is configured to be coupled to a second layer or cover layer 520 such that a cavity 550 is defined between the first layer 510 and the second layer 520 (FIGS. 3 and 4). The second layer or cover layer 520 includes an opening 522 for fluid access to the cavity 550, mechanical connection for fluid system via connection 530, a semi-rigid support base for cavity 550 and expandable wall 510 and mechanical support for the fasteners and/or cuff closure system 580 (FIGS. 14A, 14B, 14C and 12).

In some embodiments, the first layer 510 is coupled to the second layer 520 about a perimeter of the first layer 510. In other embodiments, the first layer 510 is coupled to the second layer 520 about a portion of the perimeter of the second layer 520. In another embodiment, a perimeter of the second layer 520 extends beyond the perimeter of the first layer 510. The expandable layer 510 and cover layer 520 could also be thought of, relative to the vasculature, as in inner layer (expandable layer 510) and an outer layer (cover layer 520). Alternatively, the inner layer 510 can be coupled to the outer layer 520 about a perimeter of the inner layer 510. In another embodiment, a perimeter of the outer layer 520 extends beyond the perimeter of the inner layer. Alternatively, the outer layer 520 can include a first edge, a second edge, a third edge and a fourth edge. At least one of the edges can be collocated with an edge along the perimeter of the inner layer 510.

The cover layer or second layer 520 includes a length and a width and the first layer or expandable layer 510 also includes a length and a width. In some embodiments of the device 405, the length of the first layer 510 is less than the length of the second layer 520. In another embodiment of the device 405, the width of the first layer 510 is less than the width of the second layer 520. In another embodiment, the length of the first layer 510 is sufficient for the first layer 510 to partially completely encircle a portion of a blood vessel. The length of the first layer 510 may be long enough to partially encircle, for example, a portion of the ascending aorta, the descending aorta, the superior vena cava, the inferior vena cava or a portion of a blood vessel that also includes a set of intercostal arteries or a set of intercostal veins.

In another embodiment, the length of the second layer 520 is sufficient for the second layer 520 to completely encircle a portion of a blood vessel. The second layer 520 may also include a fist end and a second end. When the second layer 520 is configured to completely encircle a portion of a blood vessel, the first end and the second end of the second layer overlap. The length of the second layer 520 may be long enough to encircle, for example, a portion of the ascending aorta, the descending aorta, the superior vena cava, the inferior vena cava or a portion of a blood vessel that also includes a set of intercostal arteries or a set of intercostal veins. The length of the second layer 510 is configured to partially encircle a blood vessel when installed about a blood vessel.

The cover layer 520 also includes at least one opening 522 in fluid communication with the cavity 550 (FIGS. 2 and 4). The cuff 405 includes a port 530 that can be coupled to the conduit 415 to deliver fluid to the cavity 550. The second layer 520 defines an opening 522 to provide fluid access to the cavity 550. A coupling 530 is provided to couple the conduit 415 to the opening 522 in the second layer 520 (FIGS. 2 and 4). The conduit 415 is coupled to the second layer or cover layer 520 in communication with the opening 522. The conduit 415 is configured to be coupled to the pump 410. As such, the conduit 415 and the fluids therein are in fluid communication with the cavity 550. In response to fluid pressure changes and/or volume changes of the cavity 550, the compliant first layer 510 is configured to deforrn (i.e., expand in response to increasing pressure or volume of the cavity 550). When the vascular assist device 405 is installed about a blood vessel (i.e., FIG. 7), the first layer 510 at least partially encircles the blood vessel. The pump and pacing controller 415 directs the pump 410 to supply fluid to the device 405 in response to and in synchronization with a signal representing the cardiac cycle of a heart in a patient body. Fluid then enters the cavity 550 causing it to increase in volume and/or pressure thus deforming the expandable wall 510. As the first layer 510 deforms (under pressure of the expanding cavity 550), the vessel encircled by the cuff 405 is compressed and blood within the vessel is urged onward. As such, the fluid (i.e., the gas or the liquid) is configured to be selectively communicated in synchronization with the cardiac cycle to the cavity 550 via a conduit 415 in communication with the opening 522 in the cover layer 520.

Embodiments of the vascular assist device of the present invention provide a compliant first layer 510 that is configured to engage internal vasculature. The second layer or cover layer 520 is coupled to the first layer 510 defining a cavity 550. The second layer 520 has a stiffness greater than a stiffness of the first layer 510. In response to changing volume of cavity 550, the first layer is configured to be deformed in response to a change in the volume of the cavity 550. Additionally, the first layer 510 is deformable such that when the pressure inside the cavity 550 increases, the first layer 510 deforms (i.e., expands). The second layer or cover layer 520 is configured to be flexible enough to encircle a blood vessel however, rigid enough not to deform under the range of pressures and volumes experienced by the cavity 550. Through the advantageous selection of the flexibility of the cover layer 520 and the expandable layer 510, the changes in fluid pressure or cavity volume are more likely to deform the expandable wall 510 and result in compression of the vessel of interest.

The advantageous functioning the cover layer and the expandable layer may be accomplished, for example, through selection of the materials selected for each of the layers. The expandable layer material may be selected to have a stiffness less than the stiffness of the cover layer. The expandable layer 510 may be fabricated with a first material and the cover layer 520 may be fabricated with a second material. In some embodiments, the first material is a first silicone elastomer and the second material is a second silicone elastomer. The first silicone elastomer may be a 5-50 A silicone elastomer having a minimum of 500% elongation. The second silicone elastomer is a 65-95 A silicone elastomer having less than a 400% elongation. In an alternative embodiment, the first material may be an elastomer having a hardness of 5-50 shore A and a minimum elongation of 500%. The second material may be an elastomer having a hardness of 65-95 shore A and a maximum elongation of 400%.

To maximize the efficiency of the device 405, the cover or second layer 520 is configured to be flexible, but does not stretch or expand under the pressure inside the cavity 550. The first layer or inner layer 510 is made of a more flexible (i.e., less stiff) material than the cover layer 520. In one particular embodiment, the inner wall or first layer 510 can be made of a 5 to 50 A silicone elastomer with a minimum of 500% elongation and the outer or cover layer 520 can be made out of less compliant silicone such as a 65 to 95 A silicone elastomer with less than 400% elongation. The first and second layers may, for example, be formed from a material that is one of silicone, neoprene and copolymers comprising styrene and butadiene. In some embodiments, the outer layer 520 is fabricated in the same manner as the first layer 510 and can be attached to the inner layer 510 by adhesives such as silicone RTV. The outer layer 520 can also be over-molded on the inner layer 510 by insert molding.

Other suitable materials for the cuff 405 (i.e., suitable materials for the layers 510 and 520) include C-Flex™, santoprene, Kraton™, PVDF, etc. Possible fabrication methods include injection molding, casting, dip molding, insert molding, over molding and blow molding. Kraton™ and C-Flex™ refer generally to thermoplastic elastomers (TPE's) that are copolymers of styrene, butadiene, and other polymers which range in hardness from 5 shore A durometer to 95 shore A durometer. C-Flex™ is commercially available from, for example, Consolidated Polymer Technologies, Inc. (CPT) of Clearwater, Fla. Kraton™ is commercially available from, for example, GLS Corporation of Delaware. Both Kraton™ and C-Flex™ are desirable materials because of their high bio-compatibility, high modulus of elasticity, and easy fabrication.

To improve the performance and durability ofthe cuff 405, the layers 510, 520 and other components in vascular assist system 400 may each be reinforced by an additional material or a reinforcement element. Reinforcement, as used herein, includes the addition of a reinforcing element to a material to prevent rupture, prevent crushing, or adjust the material properties of the material. Examples of how reinforcing elements may be used to alter the material properties of a material include the addition of reinforcing elements to alter the elongation properties of a material, reduce the permeability of a material or improve the strength of a material. In one illustrative embodiment, the second layer or cover layer includes a reinforcement element. The reinforcement element is coupled to the cover layer and configured such that the reinforcement element maintains the length and width of the cover layer as fluid is ported into and out of the cavity 550. As such, the reinforcing element is used to maintain the rigidity of the cover layer 550 so that the desired deformation of the layer 510 occurs. In this regard, the cover layer 550 provides mechanical strength for the advantageous deformation of the expanding layer 520.

In addition, the reinforcing element or elements may be incorporated into the material such that material reinforcement is selective and adjustable. Representative reinforcing materials include polyester, nylon, para-aramid fiber, stainless steel, platinum, superelastic nitinol, and alloys of nickel and titanium. The para-aramid fiber may be commercially available, such as, for example, KevlarTm, and/or polyester fibers. Alternatively, reinforcement may accomplished by simply adjusting the wall thickness a component to that the thicker wall portions of the component act as reinforcing elements. The conduits 415, 528 may also employ reinforcing elements so that the walls of the conduit do not collapse under pressure of tissue growth within the body.

The use of fiber reinforcement elements for the cover layer and/or expandable layers 510, 520 of the device 405 may also reduce the permeability of the layers 510, 520, thus reducing fluid loss through the walls. Additionally, to minimize fluid loss of the vascular assist system 400 the surfaces of the pump 410, cuff 405, and conduit 415 in contact with the fluid used in the system 400 may be coated with impermeable or semi-permeable materials such as polyethylene, polypropylene, etc. Alternatively, the inside surfaces (i.e., surfaces not in direct contact with the patient body) and/or outside surfaces (i.e., surfaces in direct contact with the patent body) of embodiments of the cuff 405, pump 410, conduits 415, 528 and the fluid volume compensator 1900 may be coated with impermeable or semi-permeable materials such as polyethylene, polypropylene, etc. to reduce fluid loss from the system 400. Metallic powder coatings can also be used for the same purpose.

The cover layer or second layer 520 extends beyond the chamber or cavity 550, thereby creating a flexible overlapping set of flaps 570. As described above the cover layer 520 provides an opening 522 and mechanical support for the attachment of coupling 530. In some embodiments of the vascular assist device 405, the cover layer 520 also provides the mechanical attachment point for the fastening means 580 used to secure the vascular assist device 405 about a portion of a vessel. In other embodiments, the vascular assist device 405 is configurable between an uninstalled configuration (i.e., when the fastening means 580 are not coupled, FIGS. 14A, 14B and 14C) and an installed configuration when the fastening means 580 are coupled (i.e., FIG. 12). In the illustrated embodiments, the cuff 405 is configurable between a first, planer configuration (FIGS. 14A, 14B and 14C) and a second configuration in which it is tubular or oval in shape and configured to be positioned around a blood vessel (i.e., a portion of a body lumen 402 as in FIG. 12). It is to be appreciated that other embodiments of the vascular assist device 405 are possible where both the first and second configurations are generally tubular and the difference between the first and second configurations depends on whether or not the fastening elements are coupled (second configuration) or uncoupled (first configuration).

The device 405 is held in position about a vessel by fastening elements 580. The flaps 570 can support the fastening elements 580 for the device 405 (FIGS. 14A, 14B and 14C). The fastening elements 580 have cooperatively configured ends 582 and 584. In the illustrated embodiment, one end 582 has a feature 585 configured to be cooperatively coupled to one of the plurality of features 586 on end 584. When the device 405 is configured about a vessel, the ends 582, 584 may be adjustably and repeatably fastened. The device 405 is adjustably fastened because the feature 585 on end 582 may be coupled to any one of the features 586 depending upon the size (i.e., external diameter) of the vessel. The device 405 is repeatably fastened because the cooperative fastening elements 585, 586 may be coupled and uncoupled repeatably. The embodiments of the vascular assist device having the adjustable and repeatable features may advantageously be employed for a wide variety of vessel sizes (i.e., diameter). A physician implanting the device 405 may install (i.e., secure about a vessel of interest) and test (i.e., activate the device by porting and removing fluid from the cavity 550) the device in a number of different configurations and positions to ensure proper fit and operation.

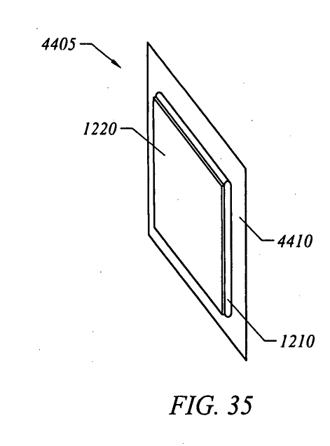

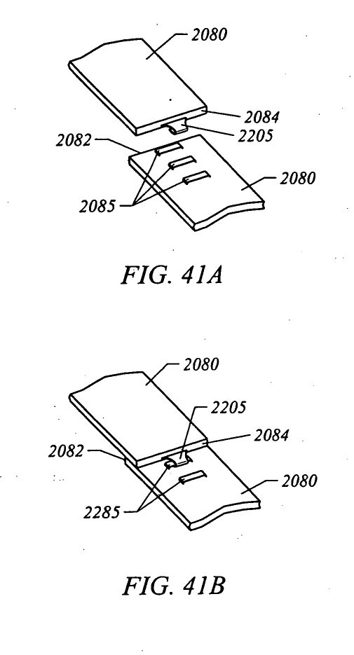

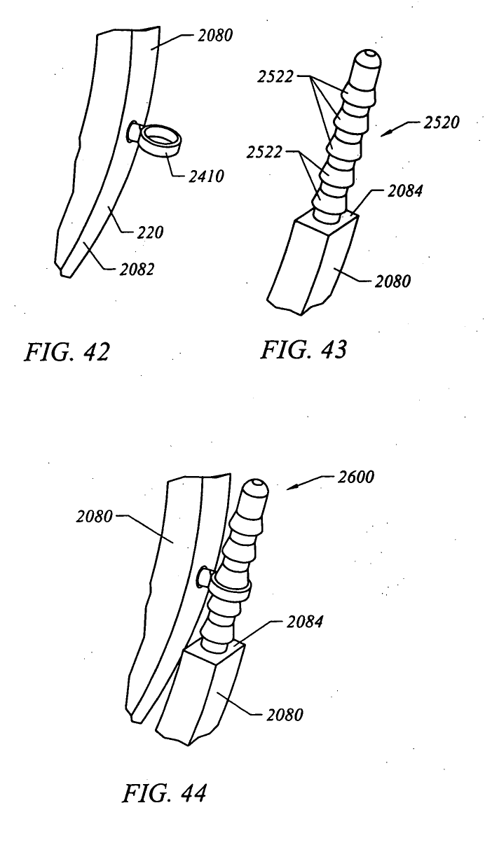

Another aspect of the adjustable quality of the fastening elements 580 is that independent attachment of the ends 582. Independent attachment refers to the ends 582 not being coupled to a correspondingly located feature 586. By reference to FIG. 2, independent attachment means that one end 582 may be attached to a feature 586 near the port 530 while the other end 582 may be attached to a feature 586 near the edge of the layer 520. Note that the left side has three attachment features 586 while the right side has four attachment features 586 with a different spacing between each attachment feature 586. The variability of the attachment features underscores the configurability of the independent attachment feature of fastening elements 580. The independent attachment feature provides an additional dimension of configurability to embodiments of the device 405. It is to be appreciated that by changing or adjusting to which of features 586 the ends 582 attach the device 405 may be configured into a wide array of shapes, such as, generally cylindrical with an adjustable diameter, or variously sized truncated conical shapes having adjustable base and apex diameters. FIGS. 14A, 14B and 14C illustrate one embodiment of a fastening element 580 for discussion purposes. Additional embodiments of the fastener elements 580 and different types of fastening are described in greater detail below with regard to FIGS. 36A-47.

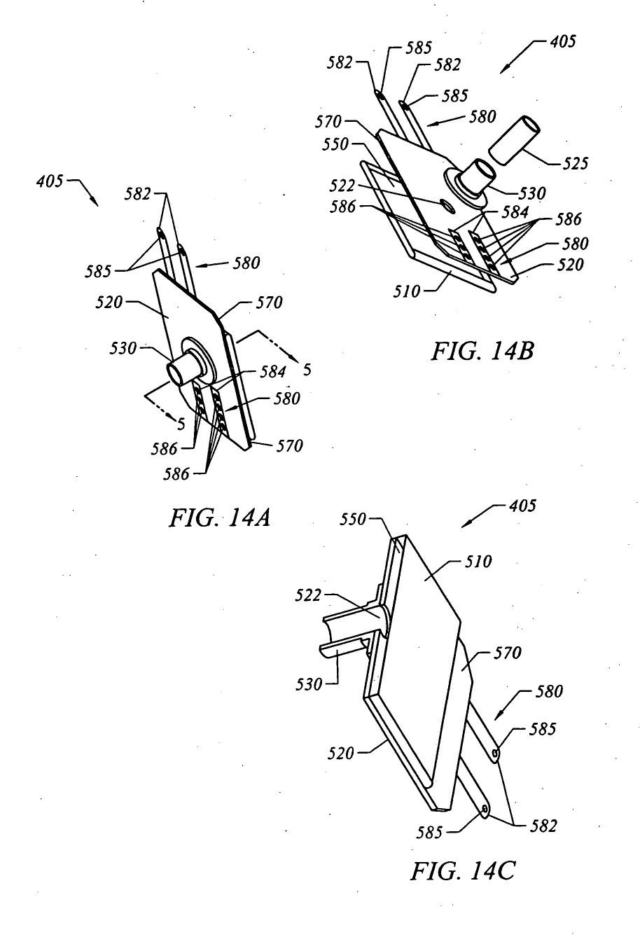

FIG. 15 illustrates a section view of an alternative embodiment of an electroactive polymer actuated pump 410′. Electroactive polymer actuated pump 410′ is situated within and provides similar functionality of electroactive polymer actuated pump 410 described above with regard to FIGS. 12, 13A and 13B. Unlike the electroactive polymer actuated pump 410, electroactive polymer actuated pump 410′ does not use a separate bladder 435 but instead the electroactive polymer layer 421 forms a cavity that contains the fluid 417. Electroactive polymer actuated pump 410′ is illustrated in an inactivated position (solid lines) and an actuated position 421′ (in phantom). Electroactive polymer actuated pump 410′ is connected to conduit 415 via coupling 411. Actuation of the electroactive polymer actuated pump 410′ results in fluid movement from the interior portion of the electroactive polymer actuated pump 410′ to the vascular assist device 405 (not shown) as indicated by arrows 419 and 421 and described above. For clarity, electroactive polymer layer 421 is illustrated as a single layer. It is to be appreciated however, that electroactive polymer actuated pump 410′ is not limited to designs having a single electroactive polymer layer 421 but includes alternative electroactive polymer actuator configurations such as, for example, a stacked electrode electroactive polymer or a multiple active area electroactive polymer actuator or any of the other electroactive polymer actuator designs described herein. The actuation of electroactive polymer actuated pump 410′ is controlled by pacing and pump controller 415 (e.g., see discussion above for EAP pump 410) or other control means to provide vascular augmentation as desired. The outer layer of the electroactive polymer layer 431a and the inner layer of the electroactive polymer layer 431b may be coated with materials to protect the functional integrity of the electroactive polymer layer 421. For example, the outer layer of the electroactive polymer layer 431a may be coated with a compound or material to induce tissue growth or protect or otherwise insulate the body from the electroactive polymer layer 421. The inner layer of the electroactive polymer layer 431b may coated with a compound or material to protect or otherwise insulate the electroactive polymer layer 421 from exposure to the working fluid 417.

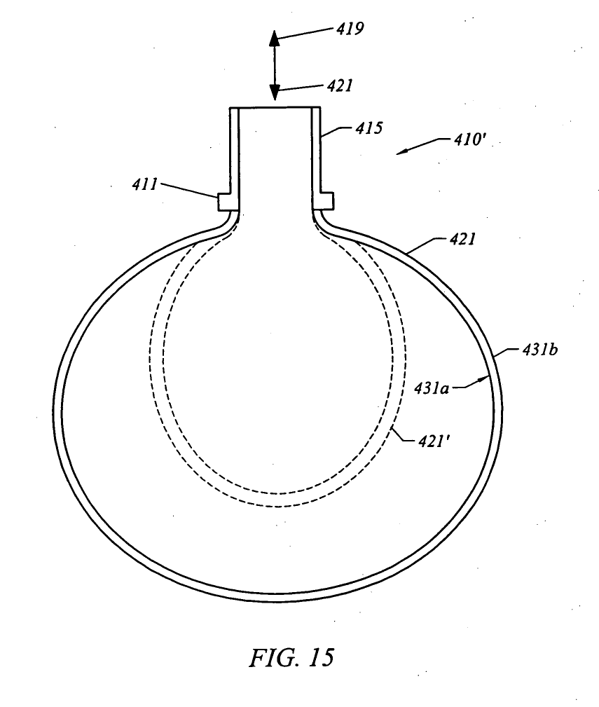

FIGS. 16A, 16B, 16C and 16D illustrate one embodiment of a single chamber, electroactive polymer actuated diaphragm pump 600. Pump 600 has a casing 605 with a connection fitting 620 having a conduit 625 in communication with the pump interior volume 635, 640. An electroactive polymer layer 610 is positioned within the casing 605 and in contact with a bias element 630. In the illustrated embodiment, the bias element 630 is a compression spring. The electroactive polymer layer 610 includes and inactive region 615 similar to the active an inactive regions discussed above in FIGS. 7B and 7D. FIG. 16C illustrates a section view along section A-A of FIG. 16A of the electroactive polymer layer in an actuated condition. When the electroactive polymer layer 610 is in an actuated condition, the bias element 630 is extended. The actuated chamber interior volume 635 is bounded by the electroactive polymer layer interior wall 611 and the casing interior wall 606. FIG. 16D illustrates a section view along section A-A of FIG. 16A of the electroactive polymer layer in an inactivated condition. When the electroactive polymer layer 610 is unactuated, the bias element 630 will pull the electroactive polymer layer 610 down into the positioned illustrated in FIG. 16D. The inactivated chamber interior volume 640 is bounded by the electroactive polymer layer interior wall 611 and the casing interior wall 606. In operation, actuation of the electroactive polymer layer 610 (starting from the condition illustrated in FIG. 16C) pushes out actuated chamber fluid volume 635 through conduit 625 to a conduit (not shown) connected to connection fitting 620 and on to an expandable cuff (see discussion of EAP actuated vascular assist system 400 above in FIG. 12). When the EAP layer 610 is in an inactivated state (FIG. 16D) the inactivated fluid volume 640 is filled by the fluid returning from the cuff (not shown) as well as the release of the stored compression force within bias element 630 (i.e., a compression spring). As discussed above, the actuation of the EAP layer 610 is done under the control of pacing and pump controller 415 to provide the desired vascular augmentation.

FIGS. 16E and 16F illustrate alternative bias arrangements from that illustrated above in FIGS. 16C, D and bias element 630. In general, a negative bias is used when the displacement of the electrode active polymer results in a reduction of chamber volume. In this case, work is done on the fluid during the time the electroactive polymer is active. The negative bias therefore, is used to return the electroactive polymer to a position that increases chamber volume. Positive bias, on the other hand, is used to impart force on the working fluid. In the case of positively biased electroactive polymer electroactive polymer actuation increases the chamber volume and the positive bias element is used to empty the chamber volume and perform work on the fluid. Bias is an important aspect of electroactive polymer design and bias is needed to ensure the electroactive polymer deflects in a predictable or designed manner, as opposed to uncontrolled deformation. Using bias to tailor the specific deflection pattern of an electroactive polymer enables the electroactive polymer to perform useful work. The bias force imparted on the electroactive polymer may be provided by any number of biasing elements such as springs, sponges or other materials that may be compressed and expanded repeatedly and reliably. Alternatively, the bias force may also be provided by the working fluid such as air, nitrogen, carbon dioxide, saline, bodily fluids, and the like. In addition, the fluid providing the bias can be a gas or a liquid. Bias force may be constant such as when a weight is placed on an electroactive polymer layer or the bias may be veritable, such as the proportional return fortune generated by a spring when a sprained is used as the bias element. Bias force may also be provided through the use of an active component, such as a bias element incorporating the use of shape memory alloys. The use of an active component such as a shape memory alloys element would allow the bias force to be altered as needed during operation of the vascular assistance assessed system by sending signals to the shape memory alloys elements to change, alter, or otherwise modify the responsiveness of the shape memory alloy bias member.

Exemplary electroactive polymer pumps using negative bias and positive bias will now be described to reference to FIGS. 16D and 16F. FIGS. 16E and 16F illustrate a chamber body 680 and an EAP layer 684 that together define a chamber volume 682 therebetween. FIG. 16E has a bias element 688 providing a positive bias force on EAP layer 684. Bias element in this illustration is a spring 688 supported by a backing plate 686. Alternatively, FIG. 16F illustrates a bias member 690 exerting a negative bias force on the EAP layer 684. In the illustrated embodiment, the bias member 690 is an open cell foam array or a sponge as used herein

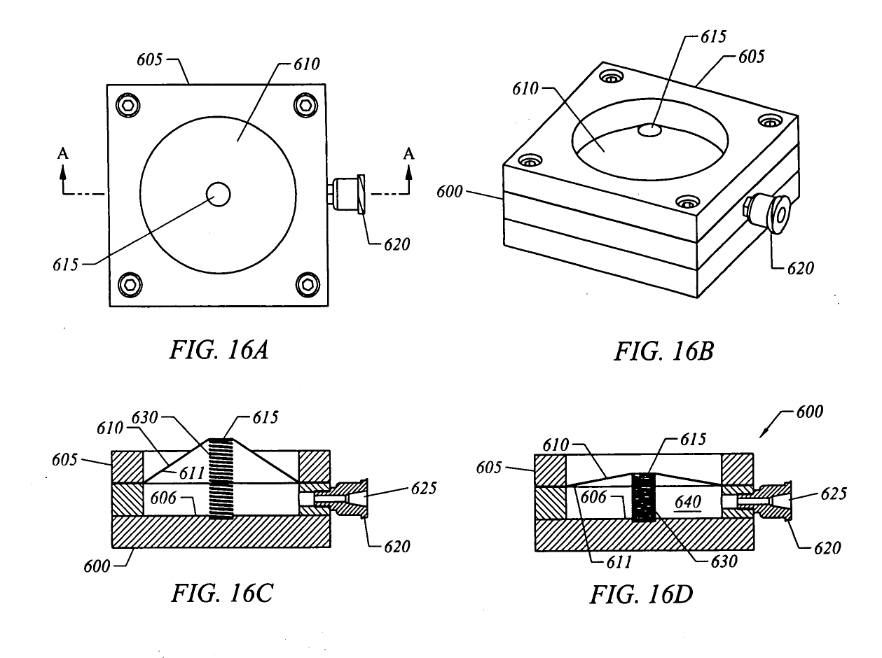

FIGS. 17A, 17B, 17C and 17D illustrate one embodiment of a single chamber, electroactive polymer actuated diaphragm pump 700. Pump 700 has a casing 705 with a connection fitting 620 having a conduit 625 in communication with the pump interior volume 735, 740. An electroactive polymer layer 710 is positioned within the casing 705. Unlike pump 600, there is no bias element. Biasing of pump 700 is provided by the return force imparted on the working fluid by the elastic forces generated as a result of the expansion of the expandable layer in the vascular assist device 405 (see FIG. 12 above). Since there is no bias element used in pump 700, the electroactive polymer layer 710 does not employ an inactive region but is instead an active region. FIG. 17C illustrates a section view along section A-A of FIG. 17A of the electroactive polymer layer in an actuated condition. When the electroactive polymer layer 710 is in an actuated condition, the actuated chamber interior volume 735 is bounded by the electroactive polymer layer interior wall 711 and the casing interior wall 706. FIG. 17D illustrates a section view along section A-A of FIG. 17A of the electroactive polymer layer in an inactivated condition. When the electroactive polymer layer 710 is unactuated, the electroactive polymer layer 710 is positioned as illustrated in FIG. 17D. The inactivated chamber interior volume 740 is bounded by is bounded by the electroactive polymer layer interior wall 711 and the casing interior wall 706. In operation, actuation of the electroactive polymer layer 710 (starting from the condition illustrated in FIG. 17C) pushes out actuated chamber fluid volume 735 through conduit 625 to a conduit (not shown) connected to connection fitting 620 and on to an expandable cuff (see discussion of EAP actuated vascular assist system 400 above in FIG. 12). When the EAP layer 710 is in an inactivated state (FIG. 17D) the inactivated fluid volume 740 is filled by the fluid returning from the cuff (not shown). As discussed above, the actuation of the EAP layer 710 is done under the control of pacing and pump controller 415 to provide the desired vascular augmentation.

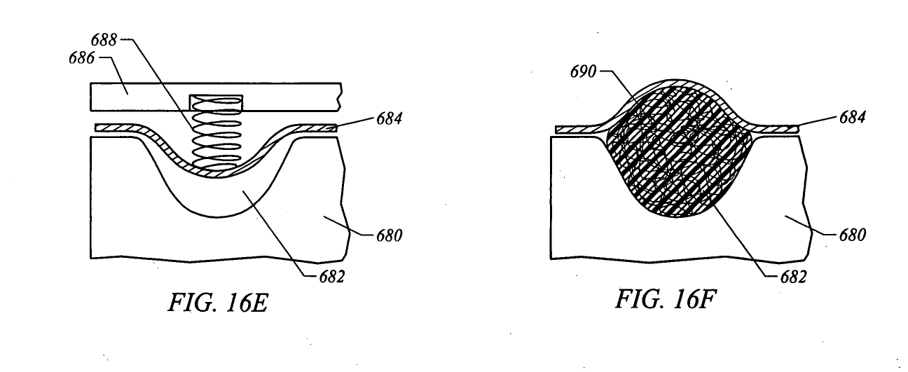

FIGS. 18A, 18B, 18C and 18D illustrate one embodiment of a dual chamber, electroactive polymer actuated diaphragm pump 800. Pump 800 has a casing 805 with a connection fitting 620 having a conduit 625 in communication with the pump interior volume 835, 840. A pair of electroactive polymer layers 810 are positioned within the casing 805. Similar to pump 700, there is no bias element. Biasing of pump 800 is provided by the return force imparted on the working fluid by the elastic forces generated as a result of the expansion of the expandable layer in the vascular assist device 405 (see FIG. 12 above). Since there is no bias element used in pump 800, the electroactive polymer layer 810 does not employ an inactive region but has instead an active region. FIG. 18C illustrates a section view along section A-A of FIG. 18A of the electroactive polymer layer in an actuated condition. When the electroactive polymer layer 810 is in an actuated condition, the actuated chamber interior volume 835 is bounded by the electroactive polymer layer interior wall 811 and the casing interior wall 806. FIG. 18D illustrates a section view along section A-A of FIG. 18A of the electroactive polymer layer in an inactivated condition. When the electroactive polymer layer 810 is actuated, the electroactive polymer layer 810 is positioned as illustrated in FIG. 18D. The inactivated chamber interior volume 840 is bounded by is bounded by the electroactive polymer layer interior wall 811 and the casing interior wall 806. In operation, actuation of the electroactive polymer layer 810 (starting from the condition illustrated in FIG. 18C) pushes out actuated chamber fluid volume 835 through conduit 625 to a conduit (not shown) connected to connection fitting 620 and on to an expandable cuff (see discussion of EAP actuated vascular assist system 400 above in FIG. 12). When the EAP layer 810 is in an inactivated state (FIG. 18D) the inactivated fluid volume 840 is filled by the fluid returning from the cuff (not shown). As discussed above, the actuation of the EAP layer 810 is done under the control of pacing and pump controller 415 to provide the desired vascular augmentation.

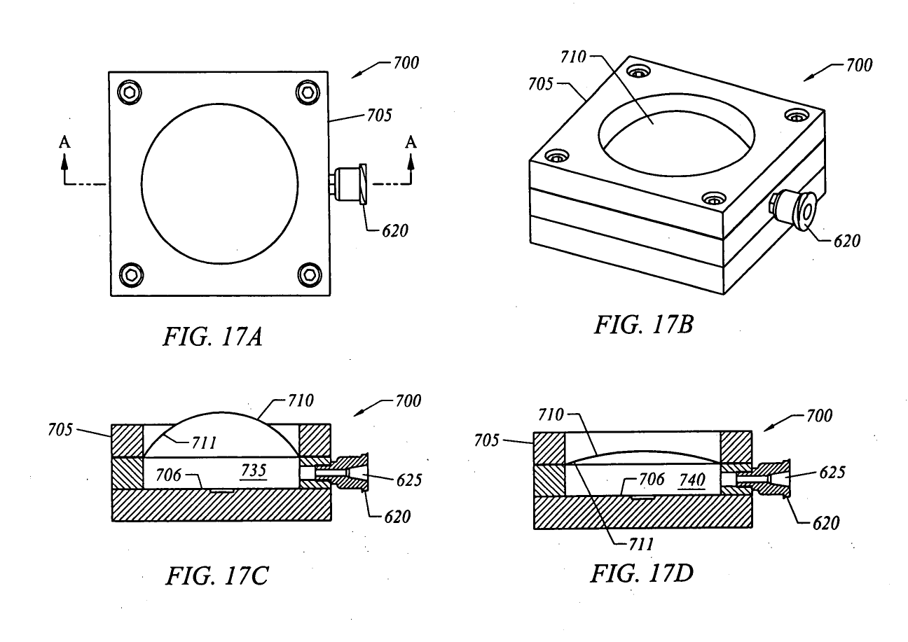

FIGS. 19A through 19D illustrate an embodiment of an electroactive polymer actuated vascular assist device according to the present invention position to augment the descending aorta (FIGS. 19A and 19B) and the ascending aorta (FIGS. 19C and 19D). FIG. 19A illustrates an embodiment of the EAP actuated vascular assist system 400 in position to augment the descending aorta 890. The EAP actuated vascular assist system 400 includes a dual chamber diaphragm pump 800 providing fluid through a conduit 415 into the cavity 550 within vascular assist device 405. Actuation of the electroactive polymer layer 810 within pump 800 (FIG. 19A) inflates cavity 550 and expands expandable layer 510 to compress the descending aorta 890. When the electroactive polymer layer 810 is deactivated, the elastic force stored in the expandable layer 510 urges the fluid out of the cavity 550 and back into the pump chamber volume 835. Additional details of the operation of an EAP actuated vascular augmentation system 400 are described above in FIG. 12 and additional details of the operation of a dual diaphragm pump are described above with regard to FIGS. 18A through 18D. For clarity, some details of the system 400 have been omitted from the above illustration such as the pacing and pump controller 415, battery 425, sensors 420 and transducer 430. Each of the omitted components operates as described above in FIG. 12.

FIGS. 19C through 19D illustrate an embodiment of an electroactive polymer actuated vascular assist device according to the present invention position to augment the ascending aorta (FIGS. 19C and 19D). In this embodiment a shorter vascular assist device 405 is used that is sized and shaped to accommodate the ascending aorta 895. FIG. 19C illustrates an embodiment of the EAP actuated vascular assist system 400 in position to augment the ascending aorta 895. The EAP actuated vascular assist system 400 includes a dual chamber diaphragm pump 800 providing fluid through a conduit 415 into the cavity 550 within vascular assist device 405. Actuation of the electroactive polymer layer 810 within pump 800 (FIG. 19C) inflates cavity 550 and expands expandable layer 510 to compress the ascending aorta 895. When the electroactive polymer layer 810 is deactivated, the elastic force stored in the expandable layer 510 urges the fluid out of the cavity 550 and back into the pump chamber volume 835. Additional details of the operation of an EAP actuated vascular augmentation system 400 are described above in FIG. 12 and additional details of the operation of a dual diaphragm pump are described above with regard to FIGS. 18A through 18D. For clarity, some details of the system 400 have been omitted from the above illustration such as the pacing and pump controller 415, battery 425, sensors 420 and transducer 430. Each of the omitted components operates as described above in FIG. 12.

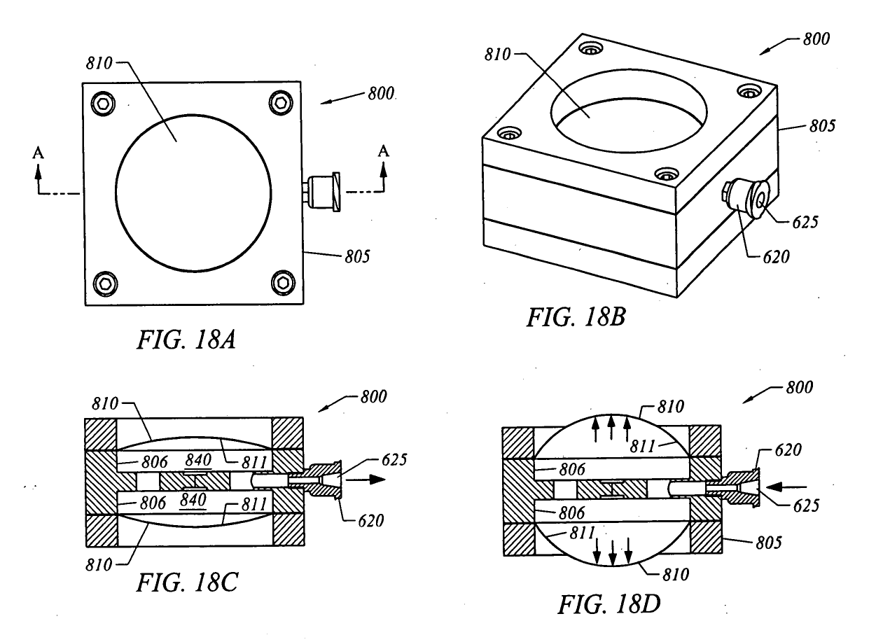

FIG. 20 illustrates an embodiment of an electroactive polymer actuated vascular assist system 400 according to the present invention implanted within a human body. As described above with regard to FIG. 12, the vascular assist system 400 includes an expandable wall assist device 405 connected to a electroactive polymer actuated diaphragm pump 800 via a conduit 415. The expandable wall assist device 405 is illustrated in a position to augment blood flow by compressing the descending aorta 890. In the illustrated embodiment of FIG. 20, sensors 420 are ECG leads that are attached to the heart 880. ECG leads 420, pump 800, and transducer 430 are electrically connected to pump and pacing controller 415. A battery pack 443 and external transducer 442 are also illustrated. The external battery pack 443 and external transducer 442 may be used to recharge an implanted power source (not shown) by capacitively coupling electrical energy from external transducer 442 to the implanted transducer 443.

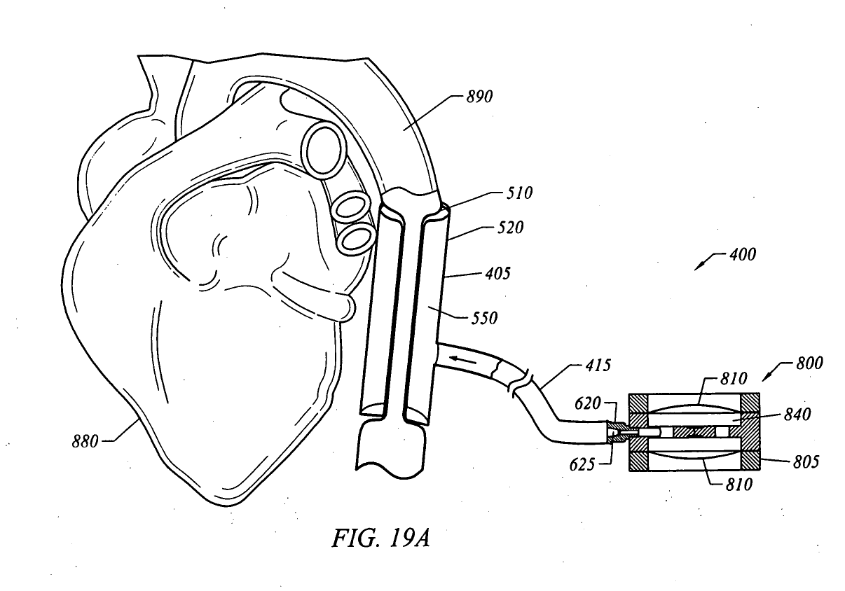

Embodiments of the EAP actuated vascular assist devices and systems of the present invention may also benefit from EAP actuated pumps having higher output volumes to drive larger or more powerful assist devices. However, in a cardiovascular assist situation, the implantable area available within the thoracic cavity places a boundary on space available to place an implantable EAP pump. In view of this need, some EAP pump embodiments of the present invention provide EAP pumps having a compact design footprints and compound or multiplied outputs. A few illustrative embodiments of output multiplied EAP pumps of the present invention will now be described through reference to FIGS. 21 through 24B.

FIG. 21 illustrates a cross section view of a multi-chamber EAP pump 900. EAP pump 900 has a body 905 having a plurality of chamber volumes 909, 910, and 911 formed therein. Each of the plurality of chamber volumes is joined by a fluid conduit 912. That is in turn, coupled to a single output 914. Similar to the design of multiple active area, the EAP 260 of FIG. 8E, a single polymer layer 915 covers all of the plurality of chamber volumes. An active polymer area 920 is created adjacent to each of the plurality of chamber volumes by placing electrode pairs 917 and 919 in proximity thereto. As described earlier with regard to multiple active area EAP 260, each of the electrode pairs 917 and 919 are individually actuable resulting in numerous actuation possibilities for the multi-chamber EAP pump 900. Each of the active areas 920 may be actuated in series, sequentially, simultaneously, or in any other combination to have the desired pump multiplication output. The actuation of the active areas 920 results in fluid movement into and out of the chamber volumes 909, 910 and 911 to produce useful work.

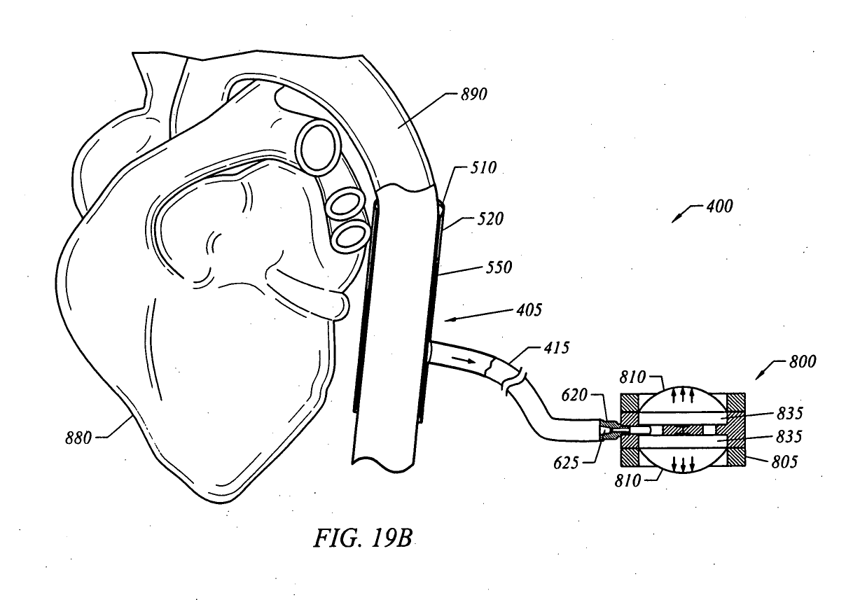

FIG. 22 illustrates a cross section view of a multi-chamber EAP pump 940. EAP pump 940 has a body 945 having a plurality of chamber volumes 946, 947, and 948 formed therein. Each of the plurality of chamber volumes is joined by a fluid conduit 954 that comprises a flow direction control means 955 such as the check valve in the illustrated embodiment. An inlet 955 allows fluids to enter the conduits 955 and chamber volumes 946, 947, and 948. Similarly, an outlet 952 allows fluids to exit under the forces generated through the actuation of EAPs 960, 962, and 964. Similar to the design of EAP actuator 130 in FIG. SA and SB, a single EAP 964, 962, and 960 is provided, respectively, above each chamber volume 948, 947, and 946. As described earlier, each of the EAP actuators 960, 962 and 964 are individually actuable resulting in numerous actuation possibilities for the multi-chamber EAP pump 940. Each of the EAP actuators 960, 962 and 964 may be actuated in series, sequentially, simultaneously, or in any other combination to have the desired pump multiplication output. Pump 940 advantageously has a single input 955 and a single output 952 with direction control means 955 thereby enabling pump 940 to operate as a continuous flow EAP actuated pump. One actuation sequence that would provide force multiplied flow would be through the sequential actuation of, for example, EAP 960 followed in order by EAP 962 and then EAP 964. It is to be appreciated that while the chamber volumes 946, 947 and 948 and EAPs 960, 962 and 964 are illustrated for purposes of discussion as having the same size, other embodiments of the EAP pumps of the present invention may have chamber volumes and EAPs of different sizes. In addition, the actuation force of each of the EAPs and the sizes of each chamber volume may change in order to provide some of the EAP pumps with relatively higher or lower force or higher or lower displacement in order that the output of EAP pump 970 may be customized. Through advantageous combinations of the use of a variety of EAPs, controlled EAP actuation and chamber volumes sizes the pump 970 may have adjustable displacement characteristics to maximize pump response time and/or flow level and/or generated output pressure.

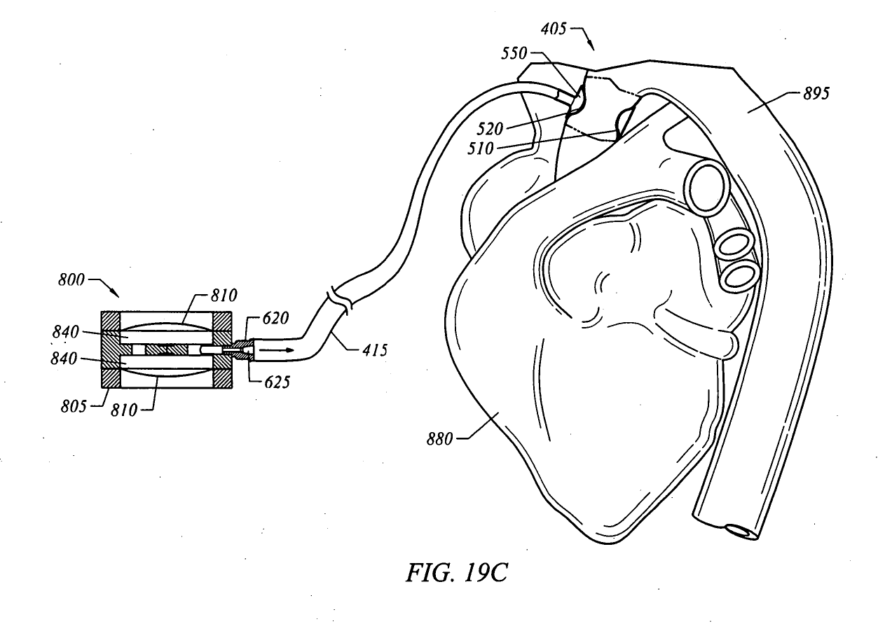

FIGS. 21 and 22 have provided two illustrative embodiments of force multiplied EAP pump embodiments having in-line or series connected EAP actuated chambers and pumps. The EAP actuated pumps of the present invention are not so limited. FIG. 23 represents a multiple chamber compound actuated EAP pump 970. EAP pump 970 includes a body 972 having a plurality of chamber volumes (not shown) but formed within the body 972 beneath each of the plurality of EAPs 984, 986, 980 and 982. The plurality of chamber volumes are connected by fluid conduits 976 to a single outlet 974. The EAP 986 is illustrated in an actuated configuration. Unlike the previously described multiple chamber EAP pumps, the EAP pump 970 has fluid conduits 976 arranged such that the chamber volume of a given EAP is in fluid communication with several other chamber volumes. Thus, the advantageous arrangement of the fluid conduits 976 provides an additional advantage for multiplying the outputs of each of the EAPs 984, 986, 980 and 982. As with other EAPs described herein, the EAPs 984, 986, 980 and 982 may be actuated in series, sequentially, simultaneously, or in any other combination to have the desired pump multiplication output.

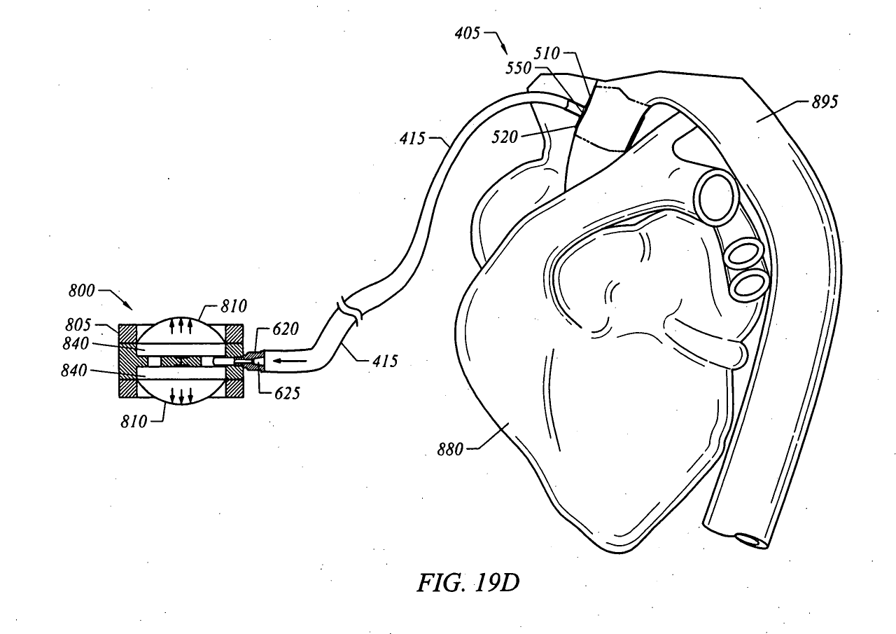

Multiple EAP actuated chamber embodiments of the present invention are not limited to the planar arrays illustrated in FIGS. 24A and 24B. Planar arrays of EAP actuated pumps may also be arranged into three-dimensional arrays. Multiple chamber compound EAP pump 1000 illustrates a plurality of vertically aligned planar arrays 1005. Each planar array includes a plurality of EAPs, chamber cavities and, if adjacent another array, a fluid coupler. The first planar array 1125 includes first layer EAPs 1110, first layer chamber cavities 1125 beneath which are found first fluid couplers 1140. The second planar array 1130 includes second layer EAPs 1115, second layer chamber cavities 1130 beneath which are found second fluid couplers 1145. The third planar array 1135 includes third layer EAPs 1120, third layer chamber cavities 1135. While the illustrated embodiment of stacked multiple chamber array EAP pump 1000 illustrates vertical coupling between the adjacent arrays, it is to be appreciated that the multiple chambers may be linked in other ways between adjacent arrays or to other EAP chambers in a single array. For example, the chamber volumes and EAPs may be linked in horizontal fashion as described above with regard to FIGS. 21 and 22. Additionally, the chamber volumes and EAPs may be cross-connected to chamber volumes in adjacent rows within a single array as described above with regard to FIG. 23. In addition, each of the EAPs within the multi-chamber pump 1000 may be actuated serially, sequentially, simultaneously or an any sequence to produce the desired pumping force multiplication. For clarity, no inlet or outlet is illustrated in pump 1000. It is to be appreciated that the complex array of pumps lends itself to numerous pumping configurations from multiple inputs to single output, single input-single output or each array may have a separate single inlet and single outlet. All of these and other inlet and outlet configurations are included within the scope of the present invention.

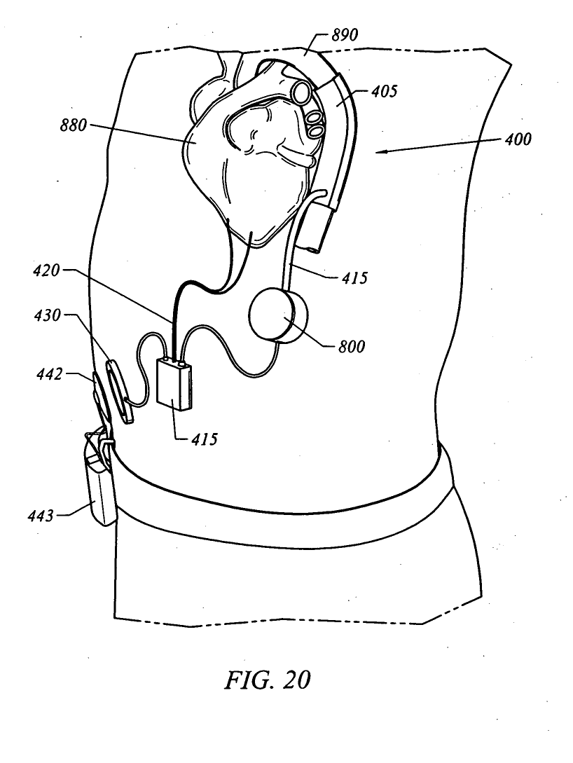

Some embodiments of EAP actuated vascular assist systems and devices of the present invention augment the fluid flow in a body lumen by directly acting on the body lumen. EAP actuated vascular assist system 1200 (FIG. 25) uses EAP based actuation to directly compress a body lumen. EAP actuated vascular assist system 1200 is similar in many regards to EAP actuated vascular assist system 400 described above with reference to FIG. 12. Common components include sensors 420, pacing and controller 415, battery 425 and transducer 430. The key difference between the two systems is EAP cuff 1202. As will be described in greater detail below, EAP cuff 1202 includes an EAP layer that is actuated under the control of pacing and controller 415 to compress the body lumen 402. EAP cuff 1202 is secured about the body lumen 402 using fasteners in the overlapping ends 1203 (described below). Actuation of the EAP cuff 1202 is accomplished using control signals transmitted via control leads 1204 that connect pacing and controller 415 to the electroactive polymer members within the EAP cuff 1202. When EAP cuff 1202 is actuated and the EAP layer deflects away from the outer wall of the cuff, a negative pressure is created between the outer wall or shell of the cuff and the deflecting EAP layer. To compensate for this change in pressure, a compliant chamber 1205 is provided. The compliant chamber 1205 is connected to the interior space between the outer wall of the cuff and the EAP layer via a conduit 1207 and a port 1208. The compliant chamber 1205 is a non-compliant or semi-compliant hollow structure that is maintained at a higher or lower or differential pressure than operating pressures that exist within the cuff during EAP layer actuation. This compliant chamber 1205 is placed in the thoracic cavity of the patient or placed in the chest or abdominal wall of the patient. In some embodiments, the compliant chamber 1205 may be eliminated by coating the shell with a highly compliant elastomeric layer.

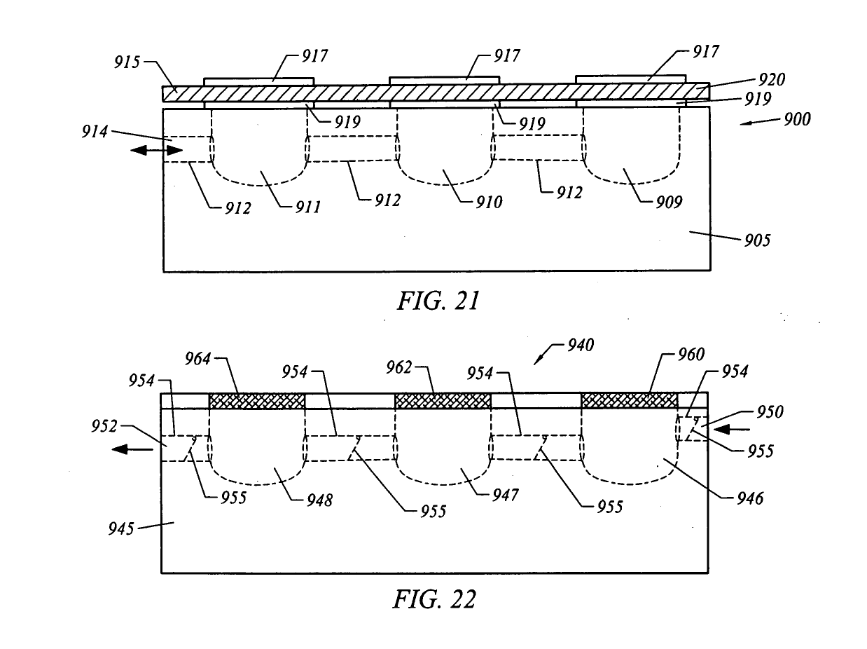

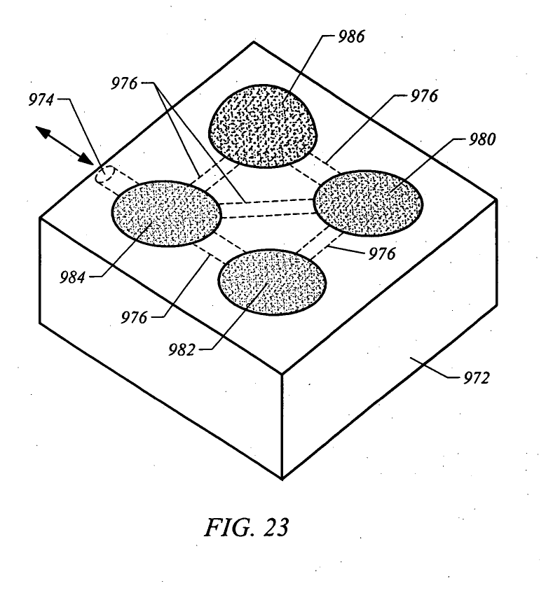

FIGS. 26A, 26B, 27A and 27B illustrate cross section views B-B of FIG. 25 of two alternative EAP layer configurations within EAP cuff 1202. The FIGS. 26A and 26B illustrate an EAP cuff 1202′ having circular EAP layer 1210. FIGS. 27A and 27B illustrate an EAP cuff 1202″ having a plurality of EAP strips 1295. FIG. 26A illustrates the actuation off condition for EAP layer 1210 within 1202′. The EAP layer 1210 is attached to the outer casing 1220 at several attachment points 1293. A flexible layer 1226 is disposed between and separates the inner wall of the EAP layer 1210 and the wall of body lumen 402. The flexible layer 1226 may be formed from any of a wide variety of flexible, compliant biocompatible materials to protect the wall of the lumen 402 from potential damage from EAP layer 1212. FIG. 26B illustrates the EAP cuff 1202′ in an actuated state. In an actuated state, the EAP layer 1210 deflects away from the outer wall 1220 and urges the flexible layer 1226 against and into compression with the wall of lumen 402. Compression of the lumen wall urges the fluid 1221 within the lumen.

Unlike EAP cuff 1202′, EAP cuff 1202″ uses a plurality of EAP strips 1295, rather then a single EAP layer 1210. EAP strips 1295 are attached between the inner wall of the outer casing 1220 and the flexible layer 1226. FIG. 27 A illustrates the EAP cuff 1202″ in a voltage off condition. FIG. 27B illustrates the EAP 1202″ in an actuated condition where each of the EAP strips 1295 has been actuated and urges the flexible layer 1226 into compression against the lumen 402. Compression of the lumen 402 results and augmentation of the flow of fluid 1221 within the lumen.

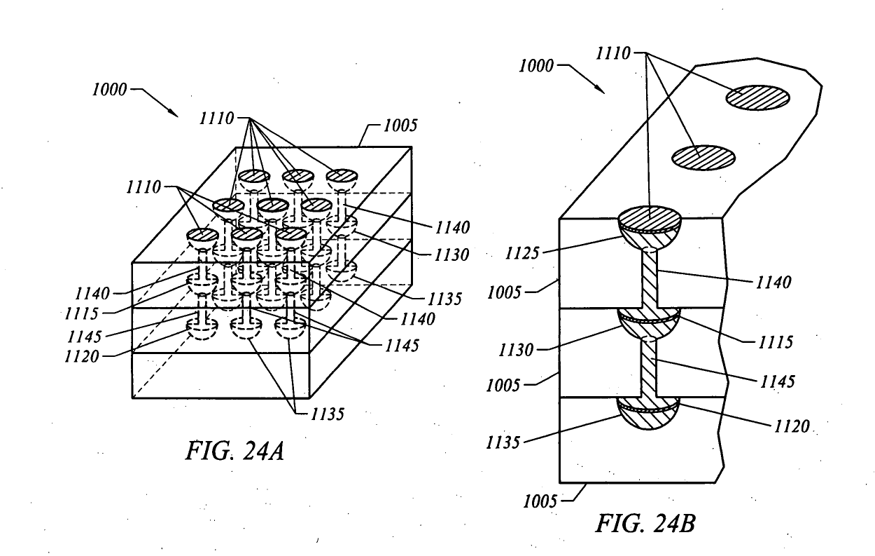

FIGS. 28A and 28B illustrate various views of an embodiment of a minimally invasive EAP actuated cuff. FIG. 28A illustrates a section view of a “C” shaped minimally invasive EAP actuated cuff 1247. Minimally invasive EAP actuated cuff 1247 is similar in design and operation to the actuator of FIG. 16F and like reference numbers will be used. The minimally invasive EAP actuated cuff 1247 includes an EAP layer 684 coupled to a base layer 680 and biased by biasing material 690 (i.e., sponge or open cell material). A strap 1287 that secures the EAP cuff 1247 in place about the lumen 402. The term “C” shape refers to the general shape formed by the backing layer 680 and the strap 1287. It is not necessary that the minimally invasive EAP actuated cuff 1247 be “C” shaped as other embodiments of the cuff 1247 will have other shapes that are sized and shaped to engage the internal vasculature of a body. The strap 1287 may utilize any of the below described removable fasteners. In the illustrated embodiment, the EAP layer 684 is in an actuated condition and compressing lumen 402. FIG. 28B illustrates a plurality of minimally invasive EAP actuated cuffs 1247 disposed along a lumen 402. In the arrangement of FIG. 28B, the plurality of minimally invasive EAP actuated cuffs 1247 may be actuated using similar system arrangements described above for actuating the EAP layer(s) 684 within each of the cuffs. Note how the use of a plurality of cuffs allows for the effective actuation of a large portion of the lumen 402. More importantly, the minimally invasive EAP actuated cuff 1247 is sized and designed for insertion about body lumens using known minimally invasive surgical techniques. For example, rather than opening the thoracic cavity to implant a single large assist device (i.e., assist device 402) a trocar may be positioned in proximity to the body lumen of interest, for example, the descending aorta, and the cuffs 1247 transitioned down the trocar and manipulated into position about the aorta (i.e., as illustrated in FIG. 28B). Using this technique, the other components of the vascular assist system may be implanted elsewhere in the thoracic cavity without having to expose the heart and aorta. While illustrated using an EAP layer 684, it is to be appreciated the other EAP layers, bias elements and arrangements are possible. For example, the EAP layer used in minimally invasive EAP actuated cuff 1247 may be an arrangement to accommodate EAP layer 1210 (FIGS. 26A and 26B) or EAP layer strips 1295 (FIGS. 27A and 27B). One important consideration for the design of minimally invasive EAP actuated cuff 1247 is for the cuff to be sized and shaped for implantation in a body about a lumen transcutaneously.

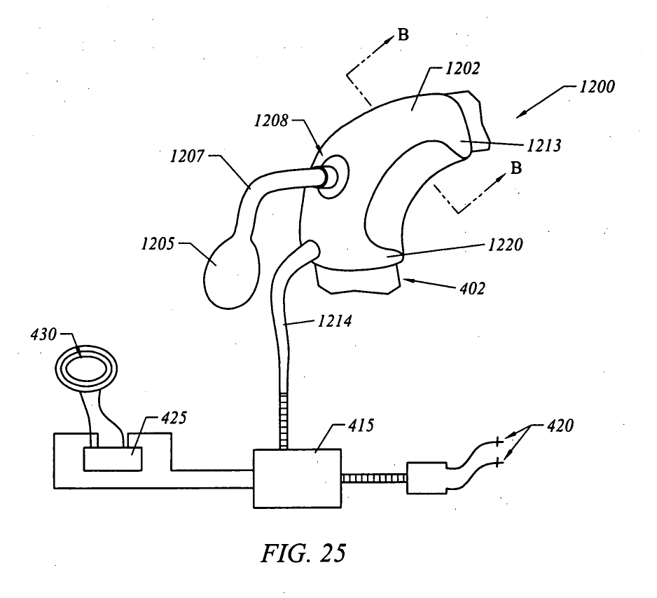

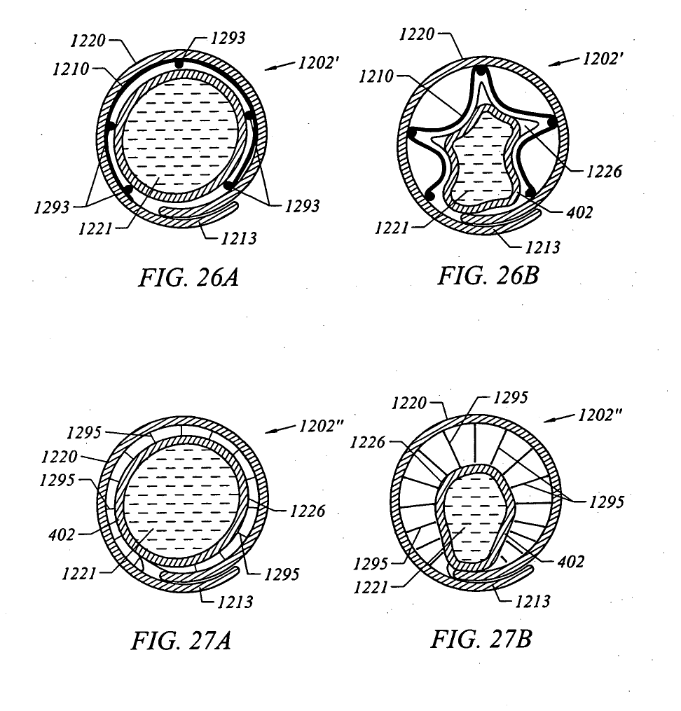

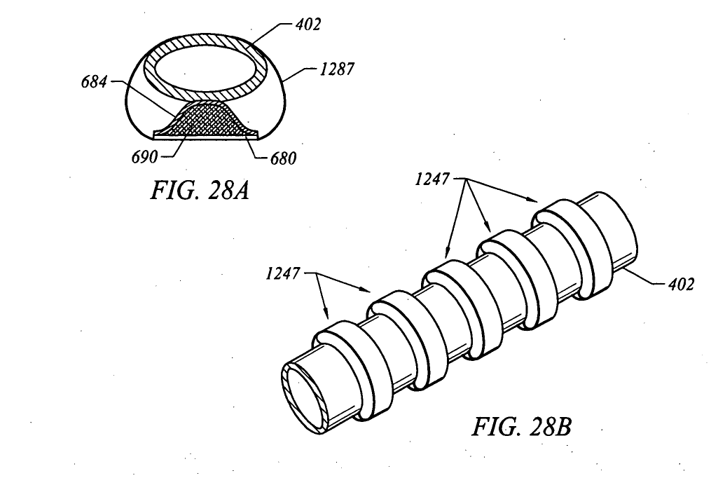

Additional details and alternative embodiments of the EAP cuff 1202 will now be discussed. FIGS. 29, 30 and 31 illustrate several views of an embodiment of the EAP cuff 1202. The cover layer or second layer 1220 is sufficiently long to surround the vasculature being augmented by the EAP cuff 1202, thereby creating a flexible overlapping set of flaps 1270. The cover layer 1220 provides mechanical support for the attachment of coupling 230 and the EAP layer 1210. In some embodiments of the EAP cuff 1202, the cover layer 1220 also provides the mechanical attachment point for the fastening means 1280 used to secure the EAP cuff 1202 about a portion of a vessel. In other embodiments, the EAP cuff 1202 is configurable between an uninstalled configuration (i.e., when the fastening means 1280 are not coupled, FIGS. 29 and 30) and an installed configuration when the fastening means 1280 are coupled (i.e., FIG. 25). In the illustrated embodiments, the EAP cuff 1202 is configurable between a first, planer configuration (FIGS. 29 and 30) and a second configuration in which it is tubular or oval in shape and configured to be positioned around a blood vessel (i.e., a portion of the ascending aorta 20 as in FIG. 25). It is to be appreciated that other embodiments of the EAP cuff 1202 are possible where both the first and second configurations are generally tubular and the difference between the first and second configurations depends on whether or not the fastening elements are coupled (second configuration) or uncoupled (first configuration).

The EAP cuff 1202 is held in position about a vessel by fastening elements 1280. The flaps 1270 can support the fastening elements 1280 for the EAP cuff 1202 (FIGS. 2, 3 and 4). The fastening elements 1280 have cooperatively configured ends 1282 and 1284. In the illustrated embodiment, one end 1282 has a feature 1285 configured to be cooperatively coupled to one of the plurality of features 1286 on end 1284. When the EAP cuff 1202 is configured about a vessel, the ends 1282, 1284 may be adjustably and repeatably fastened. The EAP cuff 1202 is adjustably fastened because the feature 1285 on end 1282 may be coupled to any one of the features 1286 depending upon the size (i.e., external diameter) of the vessel. The EAP cuff 1202 is repeatably fastened because the cooperative fastening elements 1285, 1286 may be coupled and uncoupled repeatably. The embodiments of the vascular assist device having the adjustable and repeatable features may advantageously be employed for a wide variety of vessel sizes (i.e., diameter). A physician implanting the EAP cuff 1202 may install (i.e., secure about a vessel of interest) and test (i.e., activate the EAP layer 1210) the device in a number of different configurations and positions to ensure proper fit and operation.

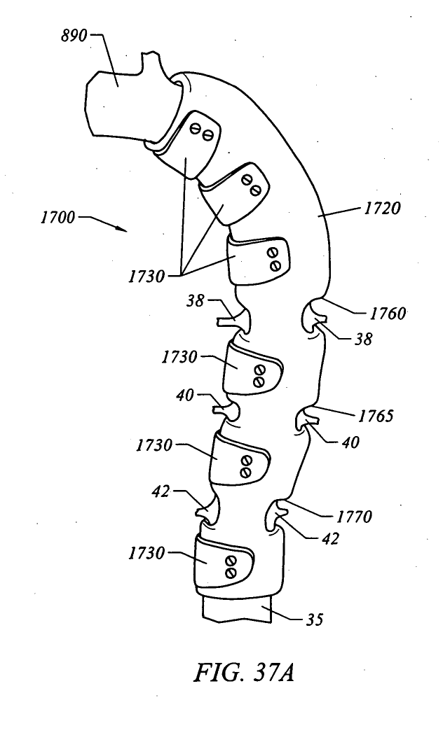

Another aspect of the adjustable quality of the fastening elements 1280 is that independent attachment of the ends 1282. Independent attachment refers to the ends 1282 not being coupled to a correspondingly located feature 1286. By reference to FIG. 29, independent attachment means that one end 1282 may be attached to a feature 1286 near the middle of layer 1220 while the other end 1282 may be attached to a feature 1286 near the edge of the layer 1220. Note that the left side has three attachment features 1286 while the right side has four attachment features 1286 with a different spacing between each attachment feature 1286. The variability of the attachment features underscores the configurability of the independent attachment feature of fastening elements 1280. The independent attachment feature provides an additional dimension of configurability to embodiments of the EAP cuff 1202. It is to be appreciated that by changing or adjusting to which of features 1286 the ends 1282 attach the EAP cuff 1202 may be configured into a wide array of shapes, such as, generally cylindrical with an adjustable diameter, or variously sized truncated conical shapes having adjustable base and apex diameters. FIGS. 29, 30 and 31 illustrate one embodiment of a fastening element 1280 for discussion purposes. Additional embodiments of the fastener elements 1280 and different types of fastening are described in greater detail below with regard to FIGS. 38-46.

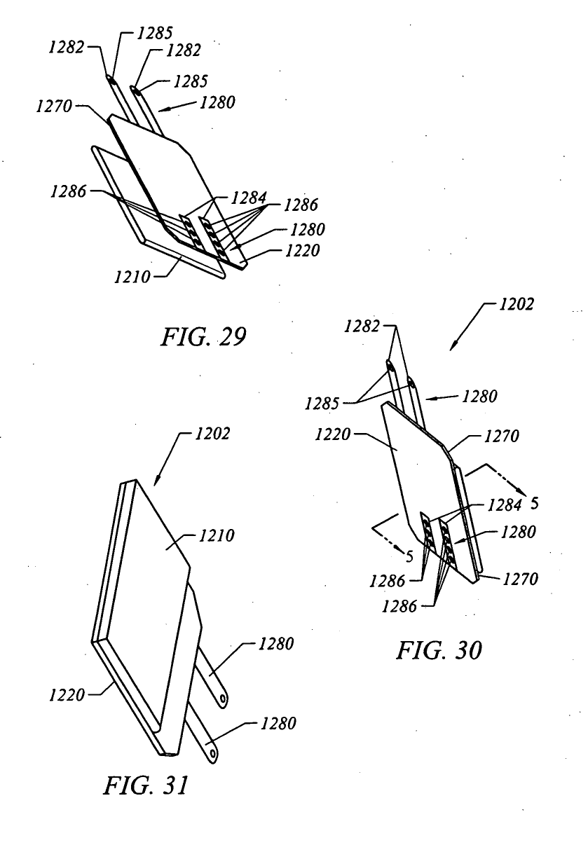

FIGS. 32A and 32B illustrate alternative embodiments of vascular assist EAP devices of the present invention. FIG. 32A illustrates a vascular assist EAP device 8500 having a cover layer 8520 and an EAP layer 8510. The cover layer 8520 has a generally rectangular shape while the EAP layer 8510 has a generally trapezoidal shape and may, advantageously, comprise multiple electrode pairs and active areas (omitted for clarity but as described above with multiple active area EAP actuator 260 in FIG. 8E). FIG. 32B illustrates a vascular assist EAP device 8550 having a cover layer 8555 and an expanding layer 8560. The cover layer 8555 has a generally trapezoidal shape and the EAP layer 8560 generally rectangular shape.

The vascular assist EAP devices 500 and 550 may also represent how embodiments of the device of the present invention may be modified to, for example, more readily engage and augment a variety of vessel types. The vascular assist EAP device 8500 illustrates a rectangular cover layer 8520 that may be an advantageous shape from the standpoint of ease for fastening the device 8500 about the vessel (FIG. 32A). The EAP layer 8510 has a trapezoidal shape having a base 8512 and an apex 8514. The trapezoidal shape may advantageously augment curved vasculature such as, for example, the ascending aorta.

Electrode placement and actuation sequence of the trapezoidal shape EAP layer 8510 may also be used to further enhance the blood flow augmentation. The vascular assist EAP device 8500 may be coupled to the fluid conduit (not shown) in a manner such that electrodes (not shown) proximate to the apex 8514 are actuated initially with subsequent electrode actuation propagating towards the base 8512. In this manner, when the vascular assist EAP device 8500 is coupled to a vessel of interest, the device 500 may be positioned so that the EAP layer actuation direction of the device (i.e., from apex 8514 towards base 510) is aligned with the direction of fluid flow in the vessel. As such, the vascular assist EAP device 8500 may be coupled to a vessel of interest in such a way that the fluid movement resulting from EAP actuation augmentation is in a direction from the apex 8514 towards the base 8512.

Alternatively, the vascular assist EAP device 8500 may be coupled to the fluid conduit (not shown) in a manner such that electrode placement and active area actuation begins proximate to the base 8512 and then propagates towards the apex 8514. In this manner, then the vascular assist EAP device 8500 is coupled to a vessel of interest, the device 500 may be positioned so that the augmentation direction of the device (i.e., from base 510 towards apex 8514) is aligned with the direction of fluid flow in the vessel. As such, the vascular assist EAP device 8500 may be coupled to a vessel of interest in such a way that the fluid movement resulting from augmentation is in a direction from advantageous electrode and active area actuation the from base 8510 towards apex 8514.

The vascular assist EAP device 8550 also illustrates how the shape of the cover layer 8555 may shaped to be more easily engaged with the vessel of interest (FIG. 32B). The cover layer 8555 has a trapezoidal shape with a base 8556 and apex 8558. The trapezoidal shape is useful in providing a wide array of non-cylindrical shapes when the edges 570 and 575 are joined together about the vessel of interest. Rectangular and trapezoidal shapes have been used with the illustrative embodiments in FIGS. 32A and 32B to illustrate these additional advantages and highly configurable nature of the vascular assist EAP devices of the present invention. Both the cover layer and the EAP layer may have other shapes, such as oval, elliptical, polygonal or irregular shapes to achieve the vessel engagement, flow augmentation, and electrode/active area actuation features described above.

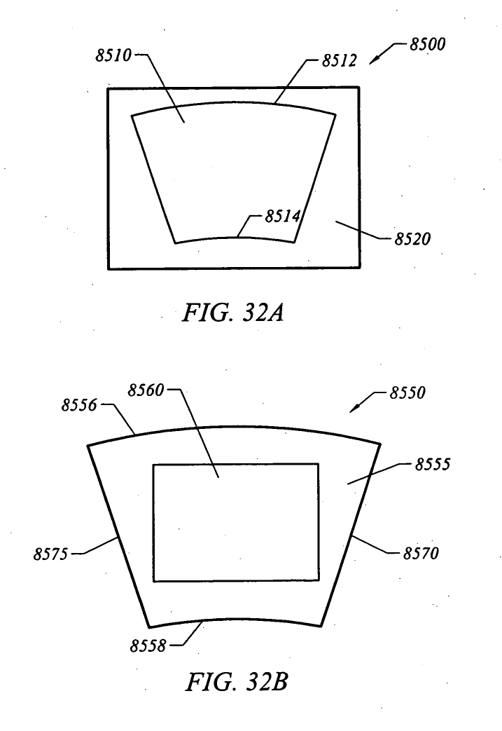

FIG. 33 is a perspective view of an embodiment of the vascular assist EAP cuff 1202 sized and in position to augment blood flow through the ascending aorta 895. The fasteners 1285 have been advantageously secured to the appropriate position on ends 1284 to ensure proper placement and fit on the ascending aorta 895.

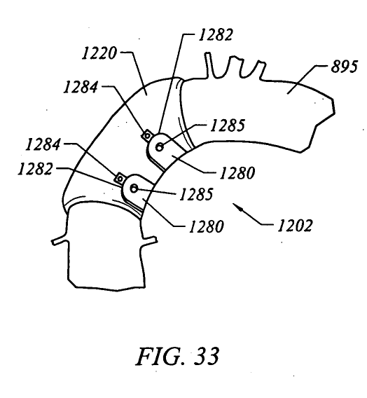

Alternative fastening means for securing EAP cuffs in position about the vasculature are possible. For example, a fabric layer 4392 may be incorporated into a vascular assist EAP device 4390 and then sutured together as the fastening means for securing vascular assist EAP device 4390 in place about a vessel (FIGS. 34A and 34B). The vascular assist EAP device 4390 is similar in all respects to the embodiments of the vascular assist EAP device 1202 described above and like reference numbers have been used. A fabric layer 4392 is incorporated into the vascular assist device 4390 between the cover layer 1220 and the EAP layer 1210 as illustrated in FIG. 34B. The fabric layer 4392 includes an end 4394 and a looped end 4393. The fabric layer 4392 may have a thickness on the order of a few microns and can be fabricated from a material such as PTFE, nylon or polyester. When the vascular assist EAP device 4390 is positioned about a vessel, the end 4394 and the looped end 4393 are sutured together thereby securing the vascular assist EAP device 4390 in place. In this way, suturing in another fastening means that may be used to secure a vascular assist device embodiment about a vessel.

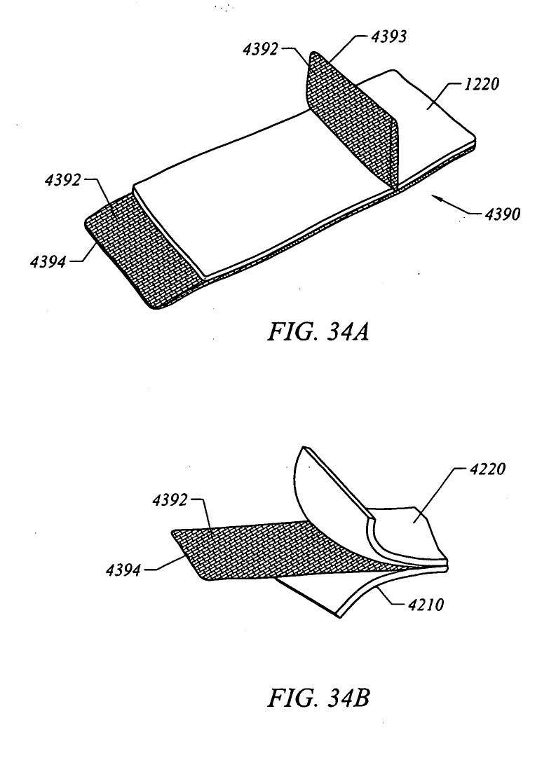

Several of the embodiments of the vascular assist EAP device of the invention have thus far been described where the EAP layer 1210 is in direct contact with the vessel to be augmented by the vascular assist EAP system. Depending on a number of factors such as, for example, vessel wall strength and the patients' physiology, there may be circumstances when another layer could be used to protect the vessel wall by being positioned between the EAP layer 1210 and the vessel wall. In some instances, the patient's vessel wall health may be less than optimal or a physician may want additional protection of the vessel from the augmentation activity of the device. In either case and for perhaps other reasons, embodiments of the vascular EAP augmentation systems of the invention can also provide a vascular engaging layer that is disposed between the EAP layer 1210 and the vessel wall. The vascular assist EAP device 4405 is one embodiment of a vascular assist EAP device of the invention that provides a vessel wall protection feature (FIG. 35). The vascular assist device 4405 is similar to the other vascular assist device embodiments described above. The vascular assist device 4405 also includes a vascular engaging layer 4410 positioned adjacent to the EAP layer 1210. The a vascular engaging layer 4410 is larger than both the expandable layer 210 and the cover layer 1220. The vascular engaging layer 4410 is bonded, affixed or other wise joined to the EAP layer 1210 such that the vascular engaging layer 4410, the EAP layer 1210 and the cover layer 1220 form a unitary structure. For example, the vascular engaging layer 4410 may be insert-molded to the EAP layer 1210. Alternatively or additionally, a primer may be applied to improve the adhesion of the vascular engaging layer 4410 to the EAP layer 1210. The vascular engaging layer 410 can have a thickness on the order of a few microns and can be fabricated from a fabric-type material such as PTFE, nylon or polyester. The vascular engaging layer 4410 may be a graft layer.

The vascular engaging layer 4410 is sufficiently long to encircle a vessel (i.e., the aorta or the vena cava). When the vascular assist device 4405 is positioned about a vessel, the vascular engaging layer 4410 encircles a vessel and is sutured together. As such, the vascular assist device 4405, like the vascular assist device 4390, employs sutures as the fastening means to secure the vascular assist device in place about the vessel of interest. While the vascular assist device 4405 illustrates an embodiment where the vascular engaging layer 4410 is integrally formed to the layer 1210, it is to be appreciated that the vascular engaging layer 4410 may advantageously employed with the other embodiments of the EAP devices described herein. For example, before an EAP cuff 1202 is installed about a body lumen, a vascular engaging layer 4410 was first fastened about the body lumen using sutures. It is to be appreciated that the vessel engaging layer 4410 or graft layer may be a separate piece from the EAP cuff 1202 or may be integrally formed with an EAP cuff by coupling it to the EAP layer. Thus, an embodiment of the vascular engaging layer 4410 may be used with any of the EAP actuated vascular assist embodiments of the present invention to achieve the vessel protection feature described above.

The embodiments of the vascular assist EAP device of the invention thus far have included continuous cuff shapes that are particularly suited to engaging and augmenting vessels having few or no protuberances or tributary vessels attached. Segmented cuffs, however, may be advantageously utilized to augment vessels having naturally occurring or artificially implanted vessels attached. Examples of naturally occurring vessels are the descending aorta with arterial intercostal and the vena cava with venous intercostal. An example of an artificially implanted vessel is the ascending aorta with a bypass graft attached thereto. In each of these cases it is desirous to augment the main vessel (i.e., aorta or vena cava) without harm to the attached vasculature (i.e., intercostal or bypass graft). The embodiments of the segmented cuffs of the present invention provide the advantages of the earlier described cuff embodiments with the added benefit of providing configurable augmentation to reduce or eliminate harm to naturally or artificially attached vasculature.

Embodiments of the segmented EAP actuated cuff of the present invention will now be described with regard to FIGS. 36A and 36B. The segmented EAP actuated cuff 1500 of the present invention is configured similar to the earlier cuff embodiments with regard to the material selection for the cover and expanding layer, fastening elements and fluid connections. The segmented EAP actuated cuff 1500 is segmented in that it includes openings or cutouts between the tabs. The specific shape of the cutout is referred to herein as the tab spacing profile. The tab spacing profile is used to configure the segmented cuff such that the cuff may wrap around a vessel of interest while not harming or obstructing flow into naturally occurring or artificially implanted vessels. Additionally, the segmented portions may also be used to avoid protuberances or other obstacles along the length of the vasculature to which the segmented EAP actuated cuff 1500 is attached. These openings or tab shape profiles are defined on opposing sides of the segmented cover layer 1520. The tab shape profiles are configured as notches or recesses defined along the opposing edges 1525 and 1530 of the segmented cover layer 1520. It is to be appreciated that embodiments of the segmented cuff are possible where the EAP layer 1510 is also segmented (i.e., multiple active areas and electrode pairs as described above). In an embodiment in which the edges of the EAP layer 1510 and outer 1520 segmented layer are coterminous, the openings or tab spacing profiles are defined in both the inner 1510 and outer 1520 segmented layers.

Returning to FIG. 36A, the segmented EAP actuated cuff 1500 includes a segmented cover layer 1520 and an expandable layer 1510 that are structurally and operationally similar to the cover layer 1220 and EAP layer 1210 described in other EAP cuff embodiments. The segmented cover layer includes a first end 1525 and a second end 1530. The first end 1525 and the second end 1530 each have at least two tabs (i.e., 1535, 1540 and 1545). In the illustrative embodiment of FIG. 14A, three tabs (i.e., 1535, 1540 and 1545) are shown. Each of the tabs (i.e., 1535, 1540 and 1545) has a width. The sum ofthe widths of all the tabs (i.e., 1535, 1540 and 1545) on one end (either end 525 or 530) is less than the width of the segmented cover layer 1520. At least two tabs on the first and second ends are configured to be removable coupled such that the segmented cuff is reconfigurable between a first configuration in which the at least two tabs on the first and second ends are separate and a second configuration in which the at least two tabs on the first and second ends are coupled. Any of the fastening elements described above or below may be provided on segmented cover layer 1520 to removeably couple the first and second ends 1525, 1530.

Another feature of the segmented EAP actuated cuff 1500 is the advantageous use of tab spacing profiles to further accommodate naturally occurring or artificially implanted vessels. Tab spacing profiles (1560 and 1570) have a width and are used to describe the spatial relationship between adjacent tabs. A tab spacing profile is used to describe the distance between the adjacent tabs (i.e., spacing profile width) and the shape of the notches formed by the tab profile between adjacent tabs. The tab spacing profile may be used to configure the resulting segmented cuff shape when the segmented cuff is implanted about a vessel. When the segmented EAP actuated cuff 1500 is installed about a vessel, the illustrative tab spacing profiles 1560 and 1570 will produce elongate rectangular segmented spaces to accommodate naturally occurring or artificially implanted vessels. It is to be appreciated that numerous tab spacing profiles are possible to accommodate a wide variety of vessel sizes and configurations. For any segmented cuff configuration the width of the segmented cuff is the sum of the widths of each of the tabs and the widths of the tab spacing profiles. For example, the width of segmented EAP actuated cuff 1500 is equal to the sum of the width of tabs 1535, 1540, and 1545 and the width of tab spacing profiles 1560 and 1570. The representative embodiment of FIG. 36A also illustrates how a variety of tab widths may be utilized in a segmented cuff. As illustrated, tab 1545 is much wider than tabs 1535 and 1540. The representative embodiment of FIG. 36A also illustrates the use of two similar tab spacing profiles. Tab spacing profile 1560 between tab 1535 and tab 1540 is the same as the tab spacing profile 1570 between tab 1540 and tab 1545.

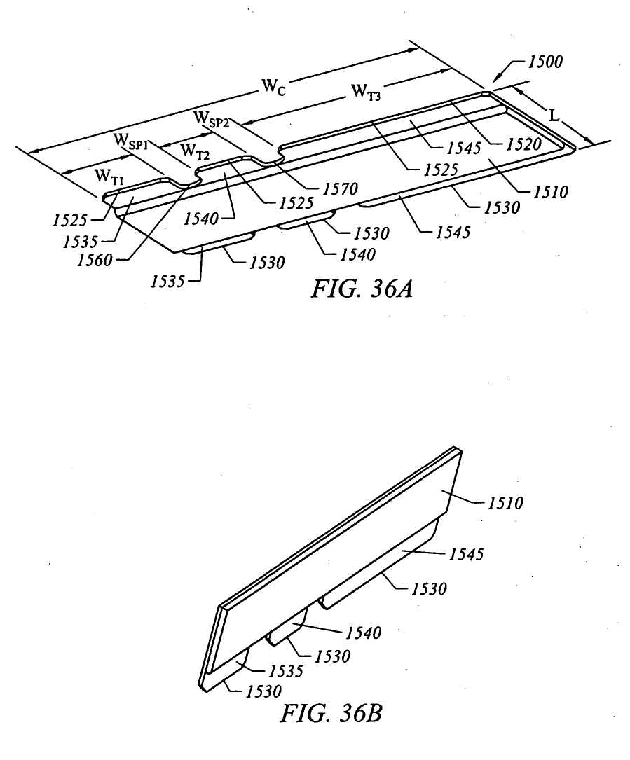

Additional advantages of the segmented EAP cuff embodiments of the present invention will be appreciated with reference to FIGS. 37A and 37B. The segmented cuff embodiments 1700 and 1850 provide additional details regarding the configurability of the EAP cuffs of the present invention and their ability to accommodate naturally occurring or artificially implanted vessels along the vessel of interest. While the applicable to artificially occurring vessels (i.e., bypass grafts) the illustrative embodiments will described and illustrated how segmented paths of the present invention may be used to accommodate naturally occurring vessels, such as, intercostal pairs 38, 40 and 42. Segmented cuff 1700 is secured in place around the descending aorta 890 using fastening elements 1730. The segmented cuff 1700 includes tab spacing profiles 1760, 1765 and 1770 to accommodate the intercostal pairs, respectively, 38, 40 and 42. Segmented EAP cuff 1700 may, advantageously, contain an EAP layer having a plurality of active areas and individually actuable electrode pairs (see EAP actuator 260 of FIG. 8E) to provide customized vessel actuation as described above with regard to FIGS. 32A. and 32B.

In contrast to the single segmented EAP cuff 1700, a group of EAP cuffs 1850 may be used to provide actuation to vessels have a natural and artificial tributaries. Like segmented EAP cuff 1700, EAP cuff group 1850 is positioned to augment the descending aorta in the vicinity of the intercostal. Here, a first EAP cuff 1830 is selected to fit on the descending aorta 890 above intercostal pair 38. A second EAP cuff 1840 is selected to fit between intercostal pairs 38 and 40. Similarly, EAP cuffs 1850 and 1860 are selected to fit between intercostal 40, 42 in the case of EAP cuff 1850 and below the intercostal 42 in the case of EAP cuff 1860. In another advantageous embodiment, EAP cuff 1830 is replaced by several EAP actuators 1247 and EAP cuffs 1840, 1850, 1860 a replaced by EAP actuators 1247 to allow for transcutaneous placement of aortic augmentation along the intercostal.

Turning now to FIGS. 38A through 47 various alternative fastener embodiments for attaching a removably coupling EAP cuffs and cuffs of the present invention about a vessel of interest will be described. As described above, fastening means 1280 is provided to secure the ends of the cover layer about the vessel of interest. When the cover layer includes a first end and a second end, the first end and the second end are configured to be removeably coupled. Thus, the vascular assist device is reconfigurable between an uninstalled configuration in which the first and second ends are separate and an installed configuration in which the first and second ends are coupled. The various anchoring, fastening, or connection mechanisms described below may be used for disposing embodiments of the cuffs of the present invention around the vasculature to be augmented. It is to be appreciated that each of the fastening means described herein allow the cuff embodiment to be moved into and out of its second or operational configuration with ease. Each of the fastening means and securing means embodiments below can be readily adjusted, repositioned and/or removed as will be described further in the discussion that follows.

The various fastening element embodiment have a number of features in common. With the exception of cuff embodiments using sewed or sutured ends (FIG. 34A and 34B), the cover layer of each cuff includes at least one pair of cooperative fastening elements. The fastening element embodiments may are repeatably configurable between an uninstalled configuration and an installed configuration. When the vascular assist device or cuff embodiment is in the uninstalled configuration, the at least one pair of cooperative fastening elements are uncoupled. When the vascular assist device or cuff embodiment is in the installed configuration, the at least one pair of cooperative fastening elements are coupled. As earlier described, one of the fastening elements in the at least one pair of cooperative fastening elements includes a plurality of fastening positions. The plurality of fastening positions are configured such that the size of the device in the installed configuration may be adjusted by changing to which of the plurality of fastening positions the other fastening element is coupled.

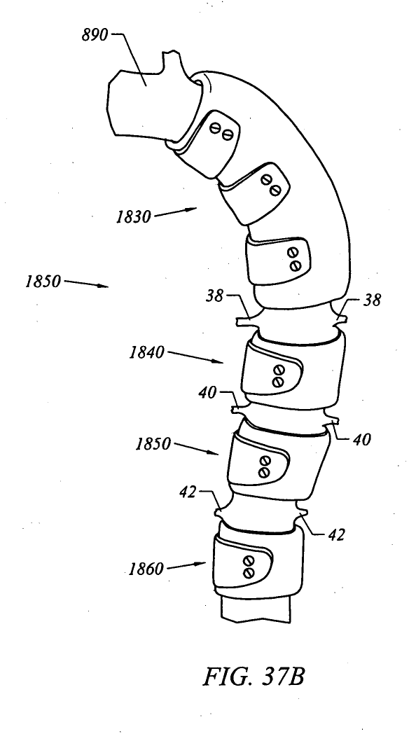

FIGS. 38A through 39B illustrate a fastener embodiment 2000 using a screw 2040 and screw receiving plate 2084 having plural positions 2085. The fastener embodiment 2000 may be attached to the flaps 1270. The ends of the fastening elements 2082, 2084 are placed into an overlapping position (i.e., ends 2082 and 2084 overlap) when the cuff is installed about a vessel (not shown) (FIG. 39A). As the end 2084 (i.e., end with the fastening plate 2087) is moved between the fastening positions 2085 on the end 2082, the size of the cuff is adjusted. When the hole 2086 is positioned above the desired receiving hole 2085, a fastener 2040 is placed through the hole 2086 and fastened to the plate 2084. The hole 2086 in the plate 2087, fastener 2040 and receiving holes 2085 are all similarly sized and threaded to operate together to secure an embodiment of the cuff about a vessel.

In the illustrated embodiment, the plate 2084 and 2087 may be metal plates integrally formed within or between layers of the fastening elements 2080. The metal strips 2084, 2087 may be stainless steel or other suitable materials such as titanium, titanium alloys, nylon, ABS, etc. The strips can be inserted in the flaps 227 during or after fabrication of the second layer 1220. To improve adhesion of the metal strips 510, 8520 to the flaps 227 of the second layer 1220, the stainless steel strips 510, 8520 can be coated with a primer.

In use, when the EAP cuff 1202 is positioned around the vessel, the appropriate opening 2085 is selected based on the size (i.e., circumference) of the vessel of interest (i.e., the aorta). A screw 2040 is inserted into the opening 2086 and threaded into the selected opening 2085. The fastener 2000 can be readily adjusted and/or removed by removing the screw 2040 and removing or repositioning the EAP cuff 1202. The screw 2040 is dimensioned such that it securely engages the threaded opening 2085, but does not extend past the cover layer. In other words, the screw 2040 does not compress the vessel.

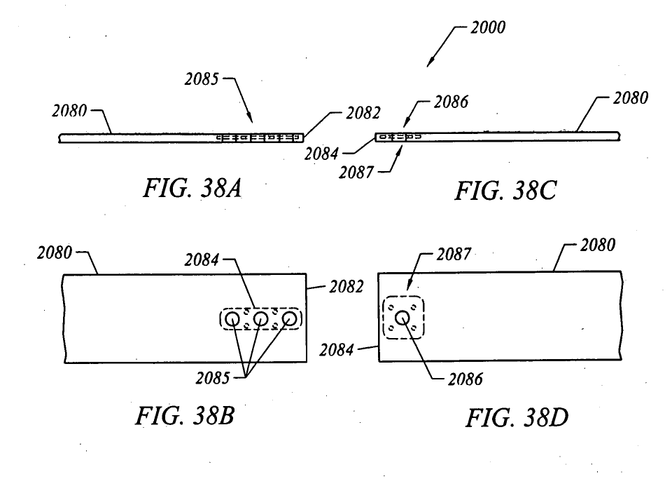

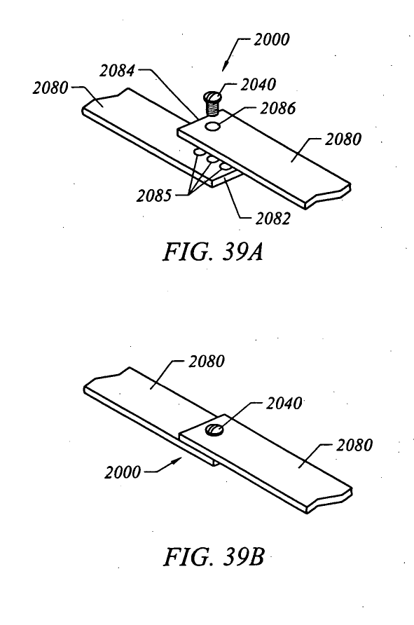

FIGS. 40A-40D and 41A and 41B are hook 2205 and anchor bars 2285 fasteners that illustrate an embodiment of a connection mechanism 2200 that can be disposed on opposing flaps 1270 described above. The connection mechanism 2200 includes at least one anchor bar 2285 in one end 2082 of the opposing flap 1270. In the illustrated embodiment, three anchor bars 2285 are illustrated. The anchor bar 21285 is a raised strip that is coupled to the second layer 1220 at two ends and defines a clearance between the anchor bar 2285 and the second layer 1220. The other flap 227 includes a metal strip 2287 with a buckle 2084 defined thereon on the other end 2084. The anchor bar 21285 and the buckle 2205 may be stainless steel or other suitable materials such as titanium, titanium alloys, nylon, ABS, etc. The anchor bar 21285 and the buckle 2205 can be inserted in the flaps 227 during or after fabrication of the second layer 1220. To improve adhesion of the anchor bar 21285 and the buckle 2205 to the flaps 227 of the second layer 1220, the anchor bar 21285 and the buckle 2205 can be coated with a primer.

In use, when the EAP cuff 1202 is positioned around the aorta, the appropriate anchor bar 21285 is selected based on the size (i.e., circumference) of the vessel. The buckle 2205 is positioned to engage the selected anchor bar 2285 through the clearance defined between the anchor bar 2285 and the second layer 1220. The connection mechanism 2200 can be readily adjusted and/or removed by disengaging the buckle 2205 from the anchor bar 2285 and removing or repositioning the EAP cuff 1202.

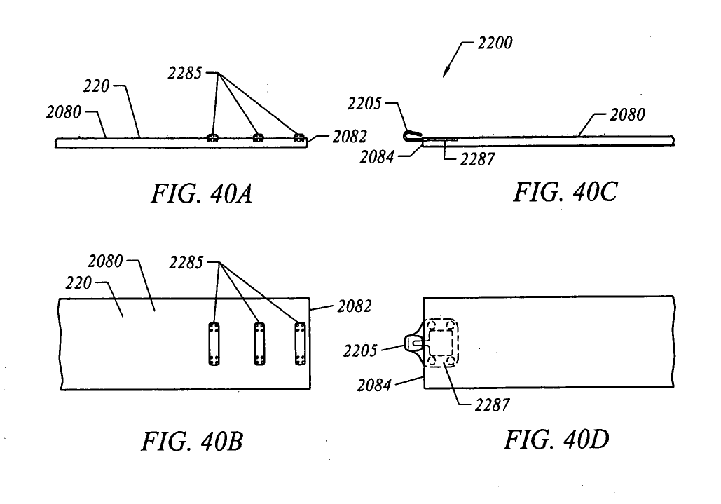

FIGS. 42, 43 and 44 illustrate an embodiment of a lock-tie wrap fastener 2600 components of the lock-tie wrap fastener 2600 can be disposed on opposing flaps 1270 described above. The connection mechanism 2600 includes a locking ring 2410 on one of the opposing flaps having end 2082. The locking ring 2410 is a raised ring that has one end embedded in the second layer 1220 of the EAP cuff 1202. The other flap 227 includes a mating element 28520 that is has multiple identical locking portions 2522. Each locking portion 2522 is configured to be pushed through the locking ring 2410, but is unable to be pulled back through the locking ring 2410. In this manner, one end 2084 with the mating element 28520 can be pushed through the other end 2082 having locking ring 240 until a secure fit is achieved. The locking ring 2410 and mating element 28520 may be stainless steel or other suitable materials such as titanium, titanium alloys, nylon, ABS, etc. The locking ring 2410 and the mating element 28520 can be inserted in the flaps 1270 during or after fabrication of the second layer 1220. To improve adhesion of the locking ring 2410 and the mating element 28520 to the flaps 1270 of the second layer 1220, the locking ring 2410 and the mating element 28520 can be coated with a primer. There is provided a cuff securing device wherein the mating fasteners include positive-locks. While the illustrative embodiment uses generally circular positive lock features, it is to be appreciated that other positive lock features are possible. The positive lock feature is the feature that holds the mating pieces in place and could have virtually any shape such as, for example, ring, square or other shape so long as holds the mating pieces into a unidirectionally oriented relationship.

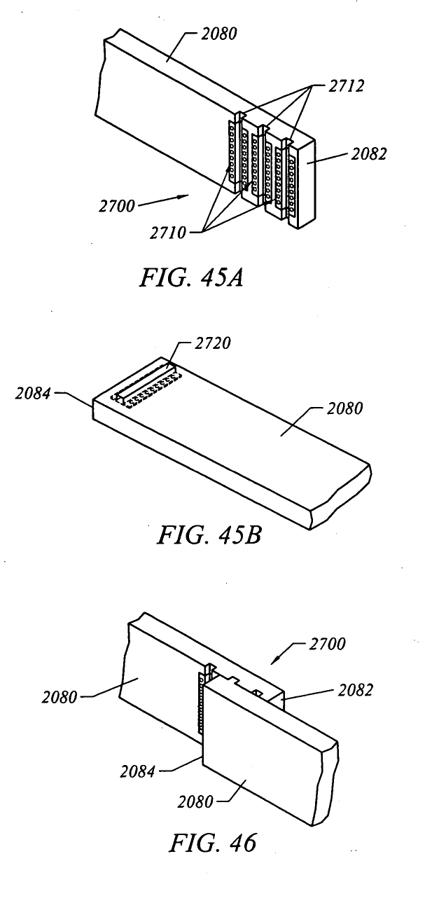

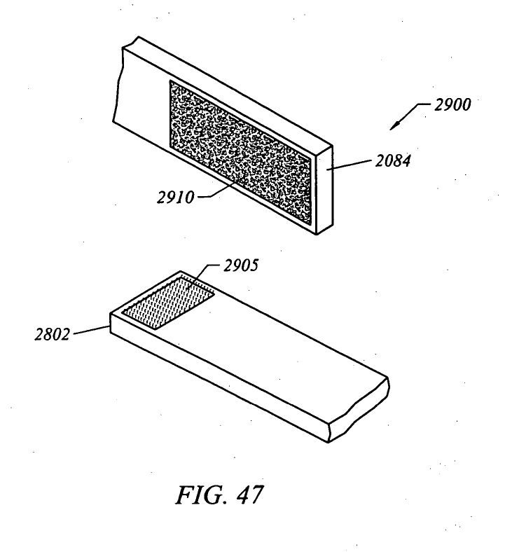

FIGS. 45A, 45B and 46 illustrate an embodiment of a connection mechanism 2700 that can be disposed on opposing flaps 227 described above. The connection mechanism 2700 includes embedded magnetic material 2710 in one of the opposing flaps. The other flap 1270 includes an embedded magnet 2720. The magnetic material 2710 and the magnet 2720 can be inserted in the flaps 1270 during or after fabrication of the second layer 1220. To improve adhesion of the magnetic material 2710 and the magnet 2720 to the flaps 1270 of the second layer 1220, the magnetic material and the magnet may be coated with a primer.

In the illustrated embodiment, the magnetic material 2710 is disposed about channels or grooves 2712 defined along the flap 2080. Moreover, the magnet 2720 is disposed externally to the opposing flap adjacent end 2084. In this manner, the magnet can engage the groove 2712 to achieve a secure coupling in which there is a greater interface between the magnetic material 2710 and the magnet 2720.

In use, when the EAP cuff 1202 is positioned around a vessel, the magnet 2720 is aligned with the appropriate groove 2712 based on the size (i.e., circumference) of the vessel. The magnet 2720 is positioned to engage the selected groove 2712 and the corresponding embedded magnetic material. The magnetic connector 12700 can be readily adjusted and/or removed by disengaging the magnet 2712 from the groove 2712 and removing or repositioning the EAP cuff 1202. Accordingly, there are embodiments of the magnetic coupler system 2700 where the cover layer 2080 includes at least one pair of cooperative magnetic fastening elements. In a representative embodiments, at least one of the mating fasteners is magnetic. In another representative embodiment, there is provided a magnetic coupling system where one of the cooperative mating fasteners is a magnet and the other mating fastener is formed from a magnetically attractive material.

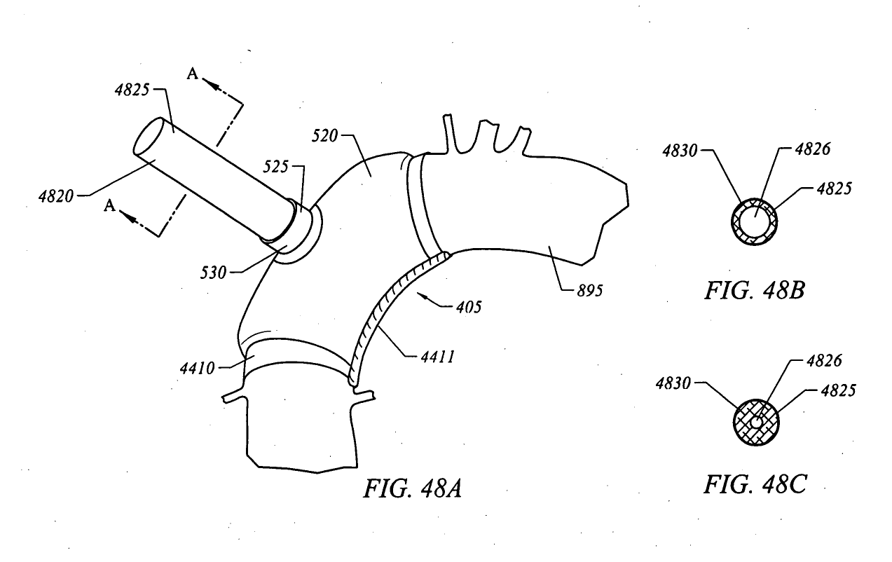

FIG. 47 illustrates an embodiment of a fastening system 2900 for use with cuff embodiments of the present invention. One flap 1270 with end 2082 includes plural fastening hooks 2905. The flap 1270 having the other end 2084 includes plural eyes or loops 2910 configured to engage with the plural hooks 2905. The plural hooks 2095 and plural loops 2910 may be, for example, strips of suitably sized Velcro™. The hook and loop material may be inserted into the flaps 227 during or after fabrication of the second layer 1220. To improve adhesion of the hook and loop material to the second layer 1220, the hook and loop material may be coated with a primer or other suitable adhesive.

In use, when the EAP cuff 1202 is positioned around a vessel, a portion of the plural hooks 2095 is aligned with the appropriate portion of the plural loops 2910 based on the size (i.e., circumference) of the vessel. The plural hooks 2095 are positioned to engage the selected portion of the plural loops 2910. The fastening system 2900 can be readily adjusted and/or removed by disengaging the plural hooks 2095 from the portion of the plural loops 2910. Thus, there is provided an embodiment of a fastener having mating fasteners that include a hook and a loop. In an alternative embodiment, the there is provided an embodiment of a fastener having mating fasteners that include a plurality of hooks and a plurality of loops.

A number of different fastener embodiments have been described. It is to be appreciated that cuff embodiments of the present invention may employ a single fastening system or multiple fastening systems to be secured about a vessel. In addition, the multiple fastening systems are not limited to including fastening elements of one type. A cuff may be secured about a vessel using two different fastening systems. In addition, the fastening systems of the present invention are not limited to the generally orthogonal orientation relative to the cover layer 1220 as illustrated in some embodiments. Fastening systems may be configured in an angular arrangement on the cover layer 1220. In some embodiments, the angular arrangement of a fastening system may be used to further conform the cover layer 1220 about the curves. Accordingly, the fastening system embodiments of the present invention may include a mixture of securing systems and angular orientations to ensure greater compliance when secured about a vessel of interest.

Rolled electroactive polymer actuators (described above in FIGS. 8A-8D and 9A-9C) may also be advantageously utilized in EAP actuated vascular assist systems of the present invention. FIG. 48A illustrates a rolled EAP actuator 4820 having a rolled EAP layer (shown in FIGS. 48B, 4C) inside of casing 4825 and defining an actuator volume 4826. Actuator volume 4826 is coupled via fittings 530, 525 to the cavity (not shown) within cuff 405. Cuff 405 is positioned on a vascular protecting layer 4410 and sutured 4411 in place on the ascending aorta 895. The rolled EAP actuator 4820 is controlled using a system similar to system 400 (FIG. 12) where EAP pump 410 is replaced by rolled EAP actuator 4820. In the illustrated embodiment, rolled EAP actuator 4820 is a radial compression rolled EAP actuator. When actuated, rolled EAP layers 4825 compress radically against the actuator volume 4826 reducing it to the size illustrated in FIG. 48C. The radial compression action of the rolled EAP 4820 (FIG. 48C) forces fluid (not shown) in the actuator volume 4826 into the cuff interior to inflate the cuff and compress the ascending aorta as described above. When rolled EAP layer 4825 shifts to a voltage off or actuation off condition, the fluid within cuff 405 is forced out by the elastic forces of the cuff to return rolled EAP layers 4825 to an inactivated state (FIG. 48B).

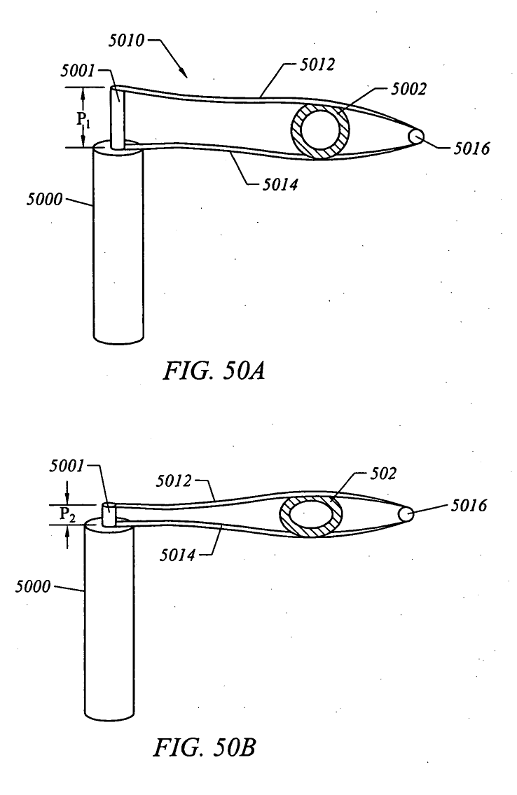

FIGS. 49A and 49B illustrate another rolled EAP actuator embodiment coupled to a cuff 405. Rolled EAP actuator 4900 has been constructed such that actuation of the EAP layers within it results in axial movement of the rolled EAP layers. For clarity the details of the interior workings of rolled EAP actuator 4900 have been omitted for clarity. One end of the rolled EAP layers is fixed to casing 4905 and the other to moveable piston 4910. When actuated, piston 4910 moves with the force of the axial deflection of the rolled EAP layers. The piston moves from its position in FIG. 49.A to its position in FIG. 49B. As the piston 4910 moves, fluid is forced into the cavity within the cuff 405, expanding the expandable layer and compressing a body lumen (not shown).

FIGS. 50A and 50B illustrate another EAP actuated vascular assist embodiment actuated by a rolled EAP actuator. Rolled EAP actuator 5000 is an axial actuation actuator similar to rolled EAP actuator 4900 (FIGS. 49A, 49B). Instead of driving a piston 4910, rolled EAP actuator 5000 is coupled to a vessel compression lever 5010. Vessel compression lever 5010 includes an arm 5012 between pivot point 5016 and the end of shaft 5001 and an arm 5014 between pivot point 5016 and the rolled EAP actuator 5000. Vessel compression lever 5010 is disposed about a body lumen 5002. When the rolled EAP actuator 5000 is actuated, arm 5014 deflects upward along shaft 5001 and compresses lumen 502. A bias spring (not shown) inside rolled EAP actuator 5000 returns the actuator and arm to position P₁, ready for the next actuation. FIG. 50C illustrates another rolled actuator 5000′ that actuates a different style of vessel compression lever 5010′ having arms 5012′, 5014′ The system moves from an actuated position (vessel 5002 compressed, in phantom) and an inactivated position (vessel 5002 uncompressed, in solid lines.)

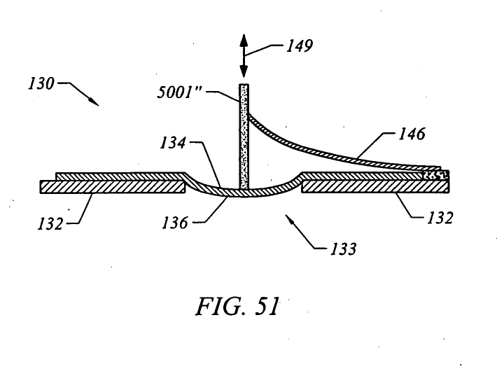

The EAP diaphragm pumps described earlier may also be used to drive a shaft coupled to a vessel compression lever, FIG. 51 illustrates an embodiment of the diaphragm pump 130 described about configured to drive as shaft 5001″ connected to a vessel compression lever (not shown but as described above with respect to FIGS. 50A-50C.)

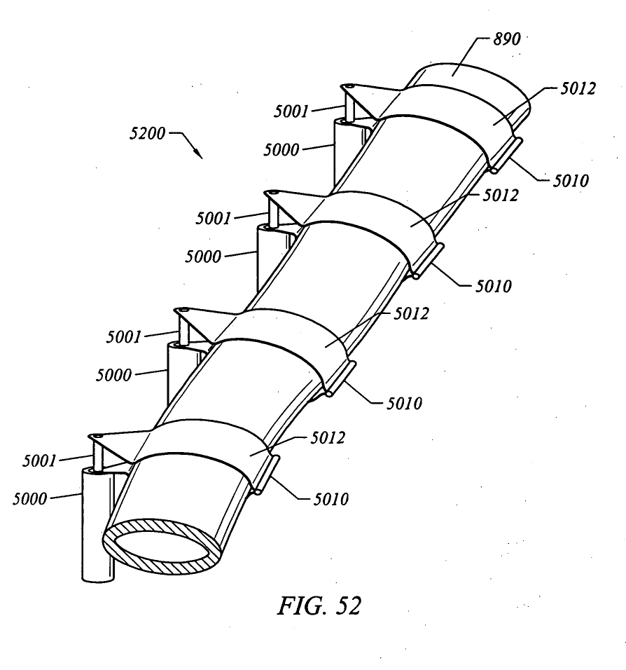

FIG. 52 illustrates an alterative embodiment ofthe rolled EAP system discussed above in FIGS. 50A and 50B. Multiple rolled EAP vascular augmentation system 5200 is similar to the systems discussed about except that the components of each rolled EAP compression system (i.e., rolled EAP actuator 5000, piston 5001 and vessel compression lever 5010) are sized and configured to be transcutaneously implanted onto the internal vasculature. As illustrated, the plurality of rolled actuators is in position to augment blood flow in the descending aorta. Each of the rolled EAP actuators may controlled using the techniques described above for actuators under the control of pacing and pump controller 415 (FIG. 12) as well as individual control for sequential, series or actuation of the actuators 5000 in any order desired.

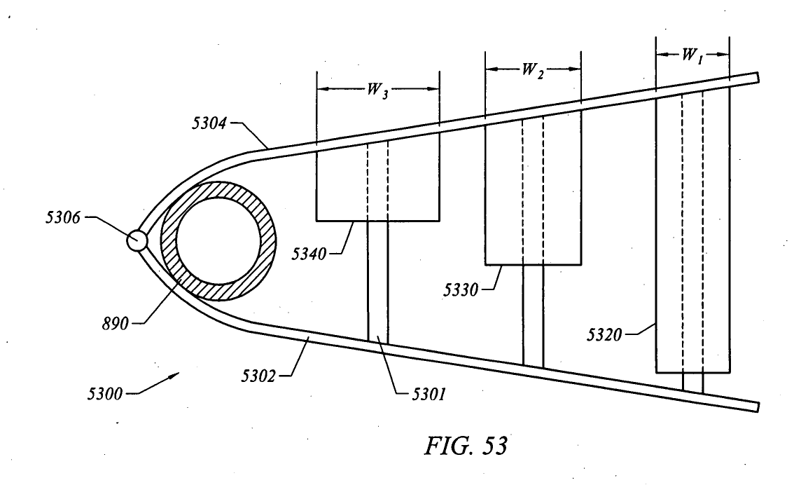

FIG. 53 represents another rolled EAP actuator vessel compression embodiment of the present invention. Rolled EAP actuator vessel compression system 5300 includes a vessel compression device 5301 with anns 5302, 5304 connected at pivot point 5306 and disposed about body lumen 890. One advantageous aspect of rolled EAP actuator vessel compression system 5300 is the use of different sized rolled EAPs 5320, 5330 and 5340. Rolled EAP 5320 is sized and shaped to have low force and large displacement. It may contain about 20 rolls of EAP layers. Rolled EAP 5330 is sized and shaped to have a higher force and lower displacement than the rolled EAP 5320. It may contain about 40 rolls of EAP layers. Rolled EAP 5340 is sized and shaped to have the highest force and lowest displacement. It may contain about 60 rolls of EAP layers. Accordingly, the size, displacement, and force profiles for each rolled EAP actuator may be adjusted depending on the number of rolls and length of the polymer layers.

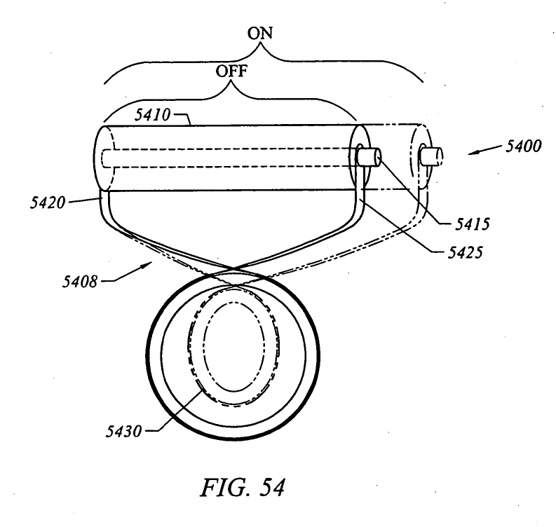

FIG. 54 illustrates another rolled EAP embodiment actuating a vessel compression device. Rolled EAP actuation system 5400 includes a rolled EAP 5410 that is connected to two arms 5420 and 5425 of a vessel compression device 5408. Rolled EAP 5410 is an axial deflecting rolled EAP. As such, when actuated the shaft end 5415 moves as indicated for the “ON” condition. As illustrated, the “ON” condition compresses the body lumen 5430 (as shown in phantom) and the “OFF” condition releases the body lumen (heavy lines). One advantage of the embodiment in FIG. 54 is than if power to rolled EAP 5410 fails, the device fails in a condition where the vessel is not compressed.

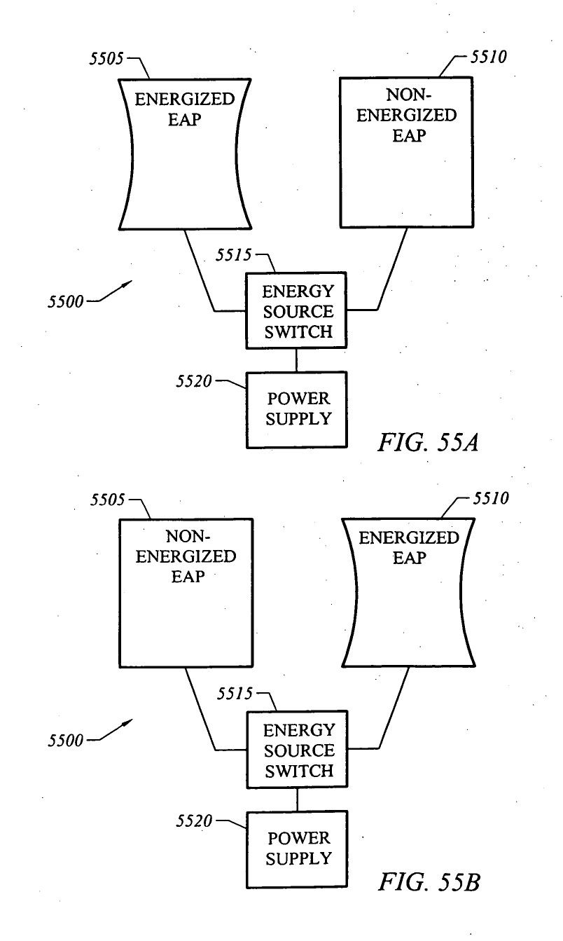

FIGS. 55A and 55B schematically illustrate an energy efficient operating scheme for high energy utilization. A generic EAP actuator system 5500 includes an opposing pair of EAP actuators 5605 and 5510 connected to an actuation power 5520 source via energy source switch 5515. One way to increase the efficiency of an EAP actuator is through the use of another capacitor or energy storage device. Here, the second storage device is another EAP actuator. Through the use of a second EAP actuator, energy may be shuttled between the two EAP actuators. FIG. 55A illustrates the case where EAP actuator 5505 is actuated and, then when it shifts to a non-energized mode (FIG. 55B), the energy stored within the EAP layers is mechanical energy that is converted back to electrical energy and transferred via energy source switch 5515 to the EAP actuator 5510 as it is being energized (shifting from FIG. 55A to FIG. 55B). By capturing and utilizing the energy occurring as a result of the elastic deflection inherent in EAP actuators, less energy is required to cyclically actuate a pair of EAP actuators that operate in concert as described above.

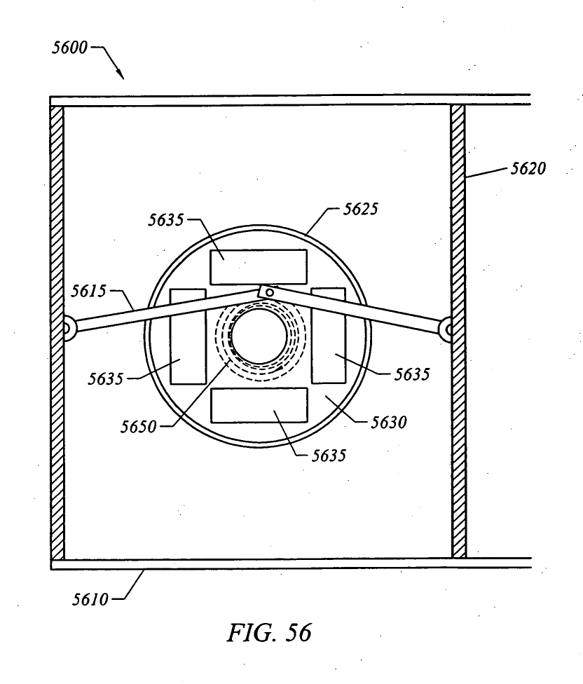

FIG. 56 illustrates a highly energy efficient EAP actuator system 5600. Highly efficient EAP actuator system 5600 includes a high efficiency EAP actuator 5625 having a polymer layer 5630 and a plurality of electrodes 5635 and active areas distributed about the polymer layer 5630. The advantageous cyclic actuation of the active areas 5635 results in the EAP layer motion lines (dashed lines 5630 in the middle of polymer layer 5630). A shaft 5615 is coupled to the central portion of the polymer layer 5630 to convert the cyclic motion of the polymer layer 5630 into mechanical energy by actuation piston 5620. As piston 5620 actuates it can be used to pump fluid that can in turn be used to actuate the inflatable cuffs of the present invention. The highly energy efficient system 5660 may be coupled to a cuff in a manner similar to the arrangement of actuation system 4900 in FIGS. 49A, 49B. Additional details are available in a previously incorporated by reference US Patent Application to Pelrine et al., “Energy Efficient Electroactive Polymers and Electroactive Polymers Devices,” U.S. patent application Ser. No. 09/779,373, filed on Feb. 7, 2001.

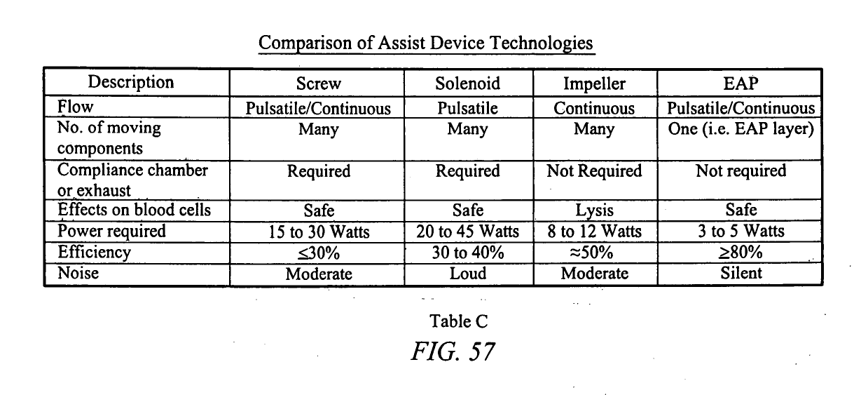

FIG. 57 contains “Comparison of Assist Device Technologies” (Table C) that compares many of the conventional vascular assist systems currently available to the EAP actuated vascular assist devices of the present invention. EAP actuated vascular assist devices have numerous advantages over the existing assist devices. Several exemplary conventional devices will now be discussed in turn. Another aspect of the EAP systems of the present invention is to provide improved EAP actuation means into conventional vascular assist systems thereby upgrading the performance and reliability of the conventional assist systems.

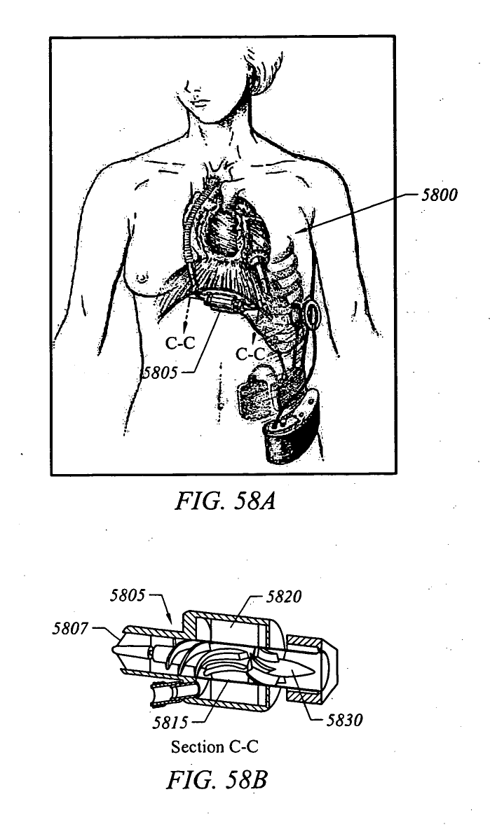

FIGS. 58A and 58B illustrate a left ventricle assist system 5800 that utilizes an impeller 5805 in contact with the blood stream to provide vascular augmentation. FIG. 58B illustrates the impeller 5805 along section C-C of FIG. 58A. The impeller 5805 includes numerous mechanically complex components such as a flow straightener 5807, inducer 5815 diffuser 5830 and motor 5820. The left ventricle assist system 5800 may be greatly simplified using any of a wide variety of EAP pumps described in this application. Replacing the screw impeller 5805 with, for example, an EAP actuated diaphragm pump (FIGS. 16, 17 and 18) or a multi-chamber EAP pump (FIGS. 21-24) would greatly simply left ventricle assist system 5800.

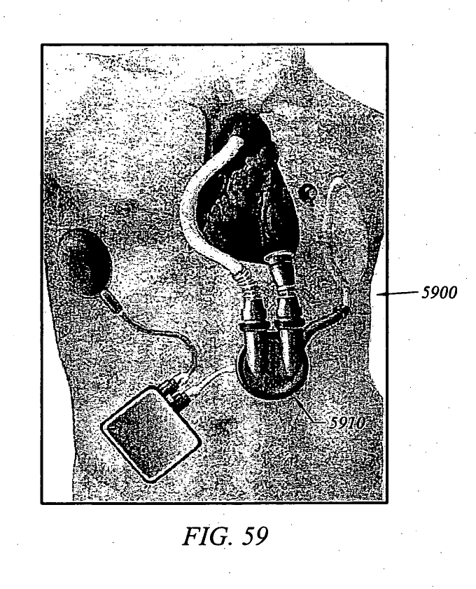

FIG. 59 illustrates a vascular assist system 5900 that utilizes a solenoid driven pump 5910 as the motive force to augment blood movement. Like the impeller 5805 discussed above, the impeller 5910 is equally as cumbersome and complicated. Similarly, vascular assist system 5900 may be greatly simplified using any of a wide variety of EAP pumps described in this application. Replacing the impeller 5910 with, for example, an EAP actuated diaphragm pump (FIGS. 16, 17 and 18) or a multi-chamber EAP pump (FIGS. 21-24) would greatly simply vascular assist system 5900.

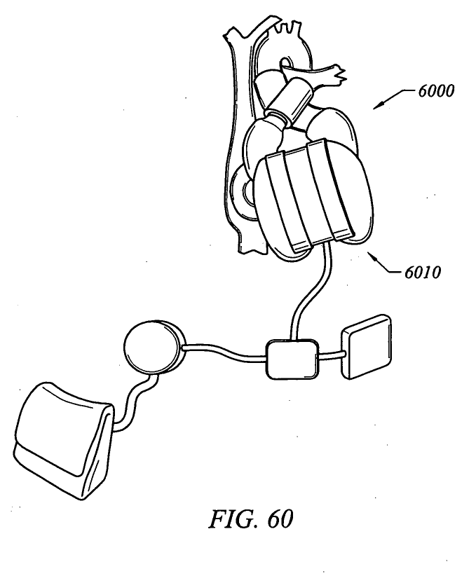

FIG. 60 illustrates a total artificial heart 6000 (TAH) and its related pumping unit 6010. Pumping unit 6010 is as complex as the above-described impellers 5805 and 5910. Like the conventional systems described above, the TAH 6000 could also be greatly improved by replacing pumping unit 6010 with an EAP actuated vascular assist system of the present invention. Similarly, vascular assist system 6000 (TAH) may be greatly simplified using any of a wide variety of EAP pumps described in this application. Replacing the pumping unit 6010 with, for example, an EAP actuated diaphragm pump (FIGS. 16, 17 and 18) or a multi-chamber EAP pump (FIGS. 21-24) would greatly simply the total artificial heart 6000.

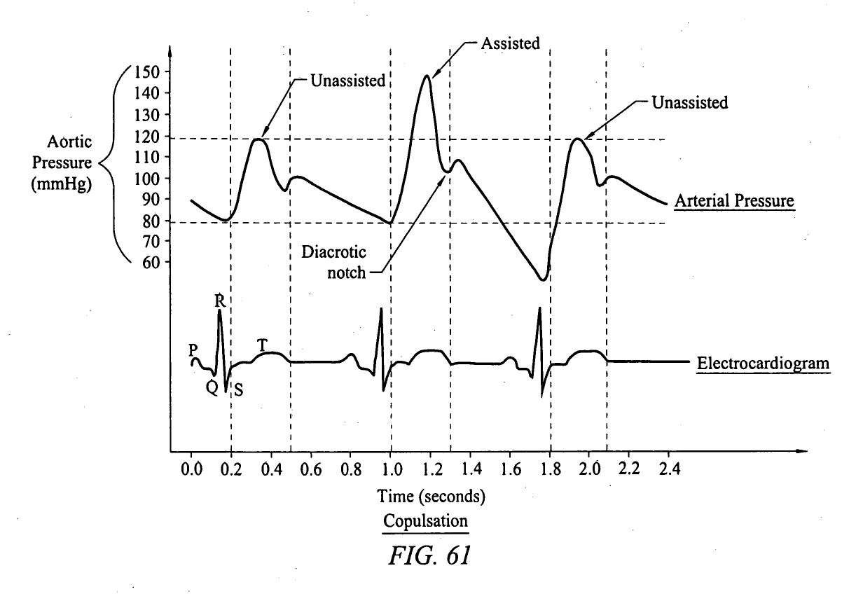

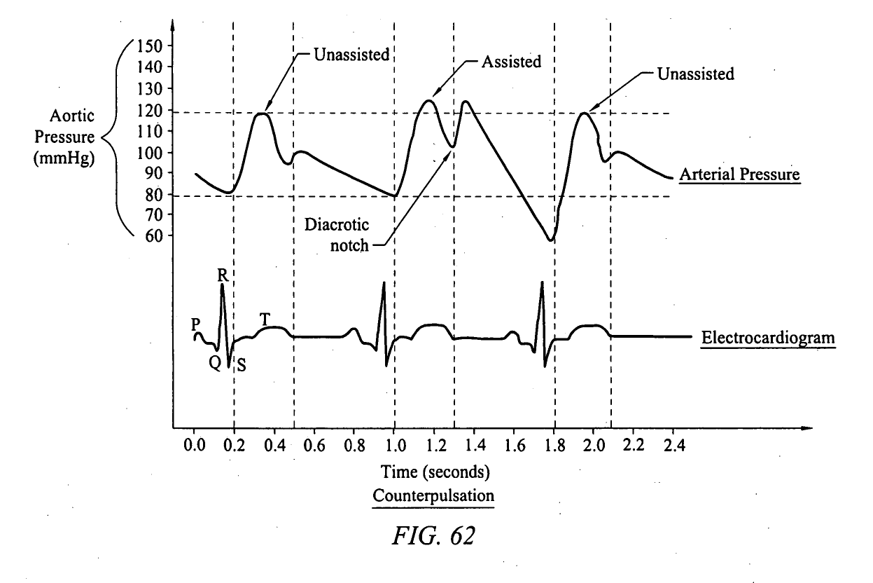

Referring now to FIGS. 61 and 62, exemplary electrocardiogram (ECG) readouts are illustrated. FIG. 61 illustrates a comparison of arterial pressure and a corresponding ECG readout when an embodiment of an EAP actuated vascular assist system of the present invention is providing augmentation is a copulsation pattern. FIG. 62 illustrates a comparison of arterial pressure and a corresponding EKG readout when an embodiment of an EAP actuated vascular assist system is providing augmentation is in a counterpulsation manner. Similar results achieved using the other embodiments of the electroactive polymer augmentation systems and devices described above.

In FIG. 61 the ECG is processed by the pacing and pump controller 415 and an R-wave is detected. Next, the pacing and pump controller 415 determines the heart rate using the R-R intervals. In order to inflate the cuff to provide copulsation, the pacing and pump controller 415 triggers the pump at about 90% rise of the R-wave. Depending on the desired dwell-time (i.e., length of time the cuff is inflated) the signal ON duration can be programmed. In this augmentation pattern, the pump shuttles the fluid from the reservoir to the cuff and inflates the cuff during the ventricular systole. In this matter, the cuff helps the heart by pushing the blood at a higher pressure. An additional benefit of this augmentation pattern is that it makes the blood flow away from the aorta faster into the side branches. When the desired dwell time (i.e., duration that cuff is inflated) has elapsed, the pacing and pump controller 320 signals for the pump to shuttle fluid back from the cuff into the reservoir (i.e., the cuff deflates). As the cuff deflates, the augmented vessel wall also relaxes. This action reduces the pressure in the aorta thus reducing the workload for the heart for the following beat.

FIG. 61 illustrates 1:2 augmentation. 1:2 augmentation means that there is one assisted heartbeat for every two unassisted heartbeats. There are three heart beats shown. First and the third heart beats (t=0.2 and t=1.8) are un-assisted and the second heart beat (t=1.0) is assisted. End-systolic pressure of the assisted beat (i.e., about 125 mm Hg) is higher compared to that of an unassisted beat (i.e., about 120 mm Hg). This increase in end-systolic pressure is known as systolic augmentation. Systolic augmentation is desired because it helps the blood flow faster at a higher pressure. The end-diastolic pressure in the second assisted beat (t=1.8, about 60 mm Hg) is lower that of an unassisted beat (t=1.0, about 80 mm Hg). This reduction in end-diastolic pressure is known as after-load reduction. As a result of after load reduction, there is less pressure in the aorta and the heart does not have to work as hard to pump the blood for the following beat. After load reduction thus reduces the workload of the heart. While the above embodiments are described using triggering based on the ECG readings, it is to be appreciated that augmentation in a co-pulsation pattern may also be triggered based on blood pressure, either venous pressure or arterial pressure.

As with FIG. 61, the ECG in FIG. 62 is processed by the pump and pacing controller 415 and an R-wave is detected. Next, the pump and pacing controller 415 determines the heart rate using the R-R intervals. In order to actuate the EAP elements to provide counterpulsation the pump and pacing controller 415 calculates the Q-T interval for the heart rate and triggers at the appropriate moment based on the response time of the EAP actuated system being used. The trigger may occur, for example, at the end of the T-wave. Depending on the desired dwell-time the signal ON duration can be programmed. An EAP actuated pump shuttles the fluid from the reservoir to the cuff and inflates the cuff during the ventricular diastole. This increases the blood flow into the coronaries and other side branch arteries. When the EAP element is deactivated, the elastic force of the cuff shuttles the fluid back from the cuff into the reservoir as the cuff deflates. This action reduces the pressure in the aorta thus reducing the work load for the heart for the following beat.

FIG. 62 shows 1:2 augmentation. There are three heart beats shown. First and the third heart beats are un-assisted (t=0.3 and t=2.0) and the second heart beat is assisted (t=1.2). Peak pressure after the diacrotic notch in the assisted beat (t=1.4, about 125 mm Hg) is greater than the peak pressure of an unassisted beat (t=0.5, less than about 100 mm Hg). This increase in secondary peak pressure provides the desired diastolic augmentation. Diastolic augmentation is desired because it increases the blood flow into the coronaries and other arteries. The end-diastolic pressure in the second assisted beat (t=1.8, about 60 mm Hg) is lower that of an unassisted beat (t=1, about 80 mm Hg). This reduction in end-diastolic pressure provides the benefits of after-load reduction as discussed above. While the above embodiments are described using triggering based on the ECG readings, it is to be appreciated that augmentation in a counter pulsation augmentation pattern may also be triggered based on blood pressure, either venous pressure or arterial pressure.

In addition, the R-R interval is calculated by a using a rolling average of R-waves based on real time heart rate changes. As the heart rates changes, so then changes the R-R interval. The pump and pacing controller 415 has software programs and electronics to record and average the R-R interval and adjust the system and cuff as needed. It is to be appreciated therefore that the augmentation patterns provided above may also advantageously utilize the rolling R-R wave averages.

As discussed above, the cuff embodiments, including EAP actuated cuffs and the EAP actuated vascular augmentation system embodiments above may be used to in a method for augmenting blood flow in a patient body. First, detect a first cardiac cycle trigger. Next, port fluid into the cavity of the cuff or actuate the cuff so as to elastically deform the first layer or otherwise compress a blood vessel in response to the first cardiac cycle trigger. Then, port the fluid out of the cavity in response to a second cardiac cycle trigger. The first cardiac is related to an ECG of the patient. Alternatively, the first cardiac trigger is related to the increasing portion of the R-wave. In another alternative embodiment, the first cardiac trigger occurs at 90% of the increasing R-wave amplitude. In another embodiment, the first cardiac trigger is related to the ECG of the patient and selected so that the step of porting a fluid into the cavity so as to elastically deform the first layer coincides with the ventricular systole. In yet another embodiment, the first cardiac trigger is related to the Q-T interval, to the decreasing portion of the T-wave or the end of the T-wave. In yet another embodiment, the first cardiac trigger is related to the T-wave and selected so that the step of porting a fluid into the cavity so as to elastically deform the first layer coincides with the ventricular diastole.

In yet another embodiment, the second cardiac cycle trigger is a predetermined time limit. In yet another embodiment, the second cardiac cycle trigger is based on the R-R interval. There is also provided an additional embodiment where the second cardiac cycle trigger is related to aortic pressure, a predetermined time limit, or is based on the R-R interval. In another embodiment, the first and the second cardiac cycle triggers are selected to operate the cuff in copulsation mode. In another embodiment, the cavity inflates during the ventricular systole of the heart. In yet another embodiment, the first and the second cardiac cycle triggers are selected to operate the cuff in counterpulsation mode.

There is also provided another method for augmenting blood flow in a body where a cardiac cycle trigger is detected. Fluid is ported into a cavity so as to elastically deform the first layer in response to the cardiac cycle trigger. The vessel is held compressed for a known duration and then fluid is ported out of the cavity in order to allow the vessel to relax. This method may utilize the cardiac trigger and augmentation modes described above.

In an alternative embodiment, the method may be performed in a copulsation manner wherein the cardiac trigger is related to the aortic pressure and selected so that the step of porting a fluid into the cavity so as to elastically deform the first layer coincides with the ventricular systole. Alternatively, the method may be performed in a counterpulsation manner, wherein the cardiac trigger is related to detecting R-wave of the ECG, computing the Q-T interval and triggering the pump to coincide with the end of the T-wave for porting the fluid into the cavity so as to elastically deform the first layer and compress the blood vessel. In yet another alternative, the method may be performed in a counterpulsation manner, wherein the cardiac trigger is related to detecting the peak aortic pressure and computing the duration for the aortic valve to close and triggering the pump for porting the fluid into the cavity so as to elastically deform the first layer and compress the blood vessel to coincide with the aortic valve closing.

In yet another alternative embodiment, there is provided a method for augmenting blood flow in a vessel of a patient that includes changing the pressure of a fluid in the cavity based on a signal associated with the cardiac cycle; deforming the first layer in response to the changing pressure of the fluid in the cavity; and deforming the walls of a vessel at least partially encircled by the first layer in response to the deforming of the first layer. This method may also utilize any of the above mentioned trigger and timing sequences described above. In addition, there is provided an embodiment where the method includes a signal associated with the cardiac cycle is related to the ECG of the patient and selected so that the step of deforming the walls of a vessel at least partially encircled by the first layer in response to the deforming of the first layer coincides with the ventricular systole. Alternatively, the changing the pressure of a fluid in the cavity is occurring so that the pressure in the cavity is increasing during the ventricular systole of the heart. Alternatively, the signal associated with the cardiac cycle is related to the T-wave and selected so that the step of changing the pressure of a fluid in the cavity coincides with the ventricular diastole. Embodiments of the present method may be operated in either or both of co-pulsation or counter pulsation mode.

In yet another embodiment, there is provided a method for augmenting blood flow in a body that includes sensing the R wave in the ECG of the body and then computing the QT interval to determine a calculated T wave. Thereafter, the calculated T wave or a signal related to the calculated T wave is used to actuate an electroactive polymer based vascular assist system. This synchronization technique may be used to actuate an electroactive polymer system to augment blood flow in a counterpulsation or co-pulsation mode. Alternatively, this synchronization technique may be used to activate an electroactive polymer system to augment blood flow during diastole or during systole. Any of a wide variety of electroactive polymer based vascular assist systems may be actuated using the synchronization technique described above. For example, in one embodiment, actuating the electroactive polymer based system augments blood flow by using electroactive polymer actuation to pump a fluid into an expanding wall cuff disposed about a body lumen. In another embodiment, actuating the electroactive polymer based system augments blood flow by using electroactive polymer actuation to compress a body lumen. In yet another embodiment, actuating the electroactive polymer based system augments blood flow by using electroactive polymer actuation to compress a deformable bladder.

In yet another embodiment, there is provided a method for augmenting blood flow in a body that includes sensing a pressure wave related to a hemodynamic pressure in the body and, based on a portion of the pressure wave, actuating an electroactive polymer based system to augment blood flow in the body. This technique may be utilized, for example, using the venous pressure or arterial pressure. This synchronization technique may also be advantageously used to activate any of the above-described electric of polymer based vascular assist systems and components. For example, in one embodiment, actuating the electroactive polymer based system augments blood flow by using electroactive polymer actuation to pump a fluid into an expanding wall cuff disposed about a body lumen. In another embodiment, actuating the electroactive polymer based system augments blood flow by using electroactive polymer actuation to compress a body lumen. In yet another embodiment, actuating the electroactive polymer based system augments blood flow by using electroactive polymer actuation to compress a deformable bladder.

### Conclusion

While various embodiments of the present invention have been described above, it should be understood that they have been presented by way of example, and not limitation. Thus, the breadth and scope of the present invention should not be limited by any of the above-described exemplary embodiments, but should be defined in accordance with the following claims and their equivalence.

The previous description of the preferred embodiments is provided to enable any person skilled in the art to make or use the present invention. While the invention has been particularly shown and described with reference to preferred embodiments thereof, it will be understood by those skilled in art that various changes in form and details may be made therein without departing from the spirit and scope of the invention.

What is claimed is:

1. A device for engaging a body lumen, comprising:
a first layer comprising an electroactive polymer and coupled to a second layer; and
the second layer having a length sufficient to at least partially encircle a body lumen and a stiffness greater than that of the first layer.

2. The device according to claim 1 wherein the electroactive polymer is a dielectric electostrictive electroactive polymer.

3. The device according to claim 2 wherein the electroactive polymer comprises a polymer selected from the group consisting of: silicone, latex, styrene, co-polymers of styrene, styrene butadiene styrene, isoprene and acrylate.

4. The device according to claim 1 wherein the electroactive polymer is an ion-exchange polymer metal composite.

5. The device according to claim 4 wherein the ion-exchange polymer metal composite comprises ionomers selected from the group consisting of: perfluorosulfate, perfluorocarboxylate, polyvinylidene fluoride and combinations thereof.

6. The device according to claim 4 wherein the ion-exchange polymer metal composite comprises electrode material selected from the group consisting of: conductive carbon, graphite, platinum, gold, silver and combinations thereof.

7. The device according to claim 1 wherein the electroactive polymer has an anode surface, a cathode surface and an elastomer material separating the anode surface from the cathode surface.

8. The device according to claim 7 further comprising an insulating layer disposed adjacent the anode surface such that the anode surface is between the insulating layer and the elastomer material.

9. The device according to claim 7 further comprising an insulating layer disposed adjacent the cathode surface such that the cathode surface is between the insulating layer and the elastomer material.

10. The device according to claim 7 wherein the anode and cathode conductivity is about 750 ohms to 1 mega-ohm.

11. The device according to claim 7 wherein the elastomer material separating the anode surface from the cathode surface dielectric strength is about 1 kV to 10 kV per mil.

12. The device according to claim 7 wherein the elastomer material separating the anode surface from the cathode surface hardness is about 3 A to 75 A durometer.

13. The device according to claim 7 wherein the elastomer material separating the anode surface from the cathode surface tensile strength is about 2 to 75 MPa.

14. The device according to claim 1 wherein the first layer is attached to the second layer forming a second layer attached portion and a second layer unattached portion wherein during activation of the first layer the first layer unattached portion is separated from the second layer.

15. The device according to claim 1 wherein the first layer is attached to the second layer forming a first layer attached portion and a first layer unattached portion wherein during first layer activation the first layer remains attached to the second layer at a single attachment point.

16. The device according to claim 1 wherein the body lumen is selected from the group consisting of: the ascending aorta, the descending aorta, a set of intercostals arteries, a set of intercostals veins, the superior vena cava, the inferior vena cava, the pulmonary vein and the pulmonary artery.

17. The device according to claim 1 wherein the second layer is shaped to fully or partially encircle a body lumen.

18. The device according to claim 1 wherein the second layer is “C” shaped.

19. The device according to claim 18 further comprising a strap having a first end and a second end is attached to the second layer across the open portion of the “C” shape.

20. The device according to claim 19 wherein the first end of the strap is attached to the second layer.

21. The device according to claim 20 wherein the second end of the strap and a portion of the second layer form cooperating portions of a mating fastener.

22. The device according to claim 21 wherein the cooperating portions of the mating fastener is selected from the group consisting of: the mating fasteners are magnets; the mating fasteners have one mating fastener that is a magnet and the other mating fastener is formed from a magnetically attractive material; the mating fasteners are opposite sides of a buckle; the mating fasteners are a screw and a screw-receiving opening; the mating fasteners are a hook and a loop; the mating fasteners comprise a plurality of hooks and a plurality of loops; the mating fasteners include a locking ring and a mating element;
and the mating fasteners include a positive-lock set.

23. The device according to claim 1 further comprising a third layer coupled to the first layer.

24. The device of claim 23, wherein the third layer is a vascular graft.

25. The device of claim 24, wherein the vascular graft is made from a polymer selected from the group consisting of: polyester, nylon, polytetrafluoroethylene and polyvinylidene fluoride.

26. The device according to claim 1 wherein a portion of the device is coated with a tissue growth inducing polymeric material.

27. The device according to claim 26 wherein the tissue growth inducing material is one of poly-L-lysine and poly-D-lysine.

28. The device of claim 1 wherein the second layer further comprises a reinforcement element configured to maintain the length and width of the second layer.

29. The device of claim 28 wherein the reinforcement element is fabricated from at least one if polyester, nylon, para-amid fiber, stainless steel, platinum, syorelastic nitinol and alloys of nickel and titanium.

30. The device of claim 1 wherein the length of the second layer is sufficient for the second layer to completely encircle a portion of a body lumen.

31. The device of claim 30 the second layer further comprising a first end and a second end wherein when the second layer is configured to completely encircle a portion of a body lumen, the second layer first end and the second end overlap.

32. The device of claim 31 further comprising a mating fastener disposed within the portion of the second layer where the first end and the second end overlap.

33. The device of claim 32, wherein the first end and the second end include cooperating portions of a mating fastener.

34. The device of claim 32, wherein the first end and the second end are configured to be sewn together.

35. The device of claim 32, wherein the mating fasteners are magnets.

36. The device of claim 32, wherein at least one of the mating fasteners is magnetic.

37. The device of claim 36, wherein a one the mating fasteners is a magnet and the other mating fastener is formed from a magnetically attractive material.

38. The device of claim 32, wherein the mating fasteners are opposite sides of a buckle.

39. The device of claim 32, wherein the mating fasteners are a screw and a screw-receiving opening.

40. The device of claim 32, wherein the mating fasteners are a hook and a loop.

41. The device of claim 40, wherein the mating fasteners comprise a plurality of hooks and a plurality of loops.

42. The device of claim 32, wherein the mating fasteners include a locking ring and a mating element.

43. The device of claim 32 wherein the mating fasteners include a positive-lock.

44. The device according to claim 31 wherein the portion of the body lumen is selected from the group consisting of: the ascending aorta, the descending aorta, a set of intercostals arteries, a set of intercostals veins, the superior vena cava, the inferior vena cava, the pulmonary vein and the pulmonary artery.

45. The device of claim 1 wherein the second layer further comprising a first end and a second end, wherein each of the first end and the second end have at least two tabs, each of the tabs in the at least two tabs has a width wherein the sum of the widths of all the tabs in the at least two tabs on the first end is less than the width of the device.

46. The device of claim 45, wherein the at least two tabs on the first and second ends are configured to be removably coupled such that the device is reconfigurable between a first configuration in which the at least two tabs on the first and second ends are separate and a second configuration in which the at least two tabs on the first and second ends are coupled.

47. The device of claim 45 wherein a tab spacing profile is provided between adjacent tabs in the at least two tabs, the tab spacing profile having a width wherein the sum of the tab spacing profile widths and the widths of all of the tabs in the at least two tabs equals the width of the device.

48. The device of claim 47 wherein the tab spacing profile between each of the tabs in the at least two tabs is the same.

49. The device according to claim 1 further comprising a sensor for detecting a signal representing cardiac rhythm and a controller to actuate the electroactive polymer layer in response to the signal.

50. A system for compressing a lumen, comprising:
a cuff having an expandable layer and a cover layer, the cover layer coupled to the expandable layer defining a cavity there between, the cavity having a volume, the cover layer defining an opening in fluid communication with the cavity; and
an electroactive polymer pump having an output in communication with the opening, wherein, the electroactive polymer pump moves a fluid to expand the expandable layer in synchronization with a portion of a cardiac cycle.

51. The system according to claim 50 further comprising a sensor for sensing a signal related to the cardiac cycle and a controller wherein the controller actuates the electroactive polymer pump in response to the signal related to the cardiac cycle.

52. The system according to claim 51 wherein the cuff, the electroactive polymer pump, the sensor, the power source and the controller are all implantable within a body.

53. The system according claim 51 wherein the cuff, the electroactive polymer pump, the sensor, induction coil, power source and the controller are all implantable within a body.

54. The system according claim 51 wherein the cuff, the electroactive polymer pump, the sensor, induction coil and the controller are all implantable within a body.

55. The system according to claim 51 herein the controller actuation results in copulsation of a portion of the cardiac cycle.

56. The system according to claim 51 wherein the controller actuation results in counterpulsation of a portion of the cardiac cycle.

57. The system of claim 50, the cover layer further comprising a first end and a second end the ends having a pair of mating fasteners selected from the group consisting of: the mating fasteners are magnets; the mating fasteners have one mating fastener that is a magnet and the other mating fastener is formed from a magnetically attractive material; the mating fasteners are opposite sides of a buckle; the mating fasteners are a screw and a screw-receiving opening; the mating fasteners are a hook and a loop; the mating fasteners comprise a plurality of hooks and a plurality of loops; the mating fasteners include a locking ring and a mating element; and the mating fasteners include a positive-lock set.

58. The system according to claim 50 wherein the cuff is sized to partially encircle a lumen selected from the group consisting of: the ascending aorta, the descending aorta, a set of intercostal arteries, a set of intercostal veins, the superior vena cava, the inferior vena cava, the pulmonary vein and the pulmonary artery.

59. The system according to claim 50 wherein the cuff is sized to completely encircle a lumen selected from the group consisting of: the ascending aorta, the descending aorta, a set of intercostal arteries, a set of intercostal veins, the superior vena cava, the inferior vena cava, the pulmonary vein and the pulmonary artery.

60. A device for compressing a lumen in a body comprising:
a cuff having a complaint layer and a semi-compliant layer coupled to the compliant layer so as to form a cavity there between; and
an electroactive polymer pump in communication with the cavity.

61. A device according to claim 60 wherein the electroactive polymer pump further comprises an electroactive polymer covering a chamber wherein actuation of the electroactive polymer causes the volume of the chamber to change.

62. The device of claim 61 wherein the electroactive polymer pump has a single chamber.

63. The device of claim 61 wherein the electroactive polymer pump has more than one chamber.

64. The device of claim 62 or 63 having a positive bias.

65. The device of claim 62 or 63 having a negative bias.

66. The device of claim 62 or 63 having a mechanical bias.

67. The device of claim 62 or 63 having a pressure differential bias.

68. The device according to claim 63 wherein each one of the more than one chamber is connected serially to at least one of another of each one of the more than one chamber.

69. The device according to claim 63 wherein each one of the more than one chamber is connected in parallel to at least one of another of each one of the more than one chamber.

70. The device according to claim 63 wherein each one of the more than one chamber is connected to another one of the more than one chamber using a combination of parallel and serial connections.

71. The device of claim 63 wherein the chambers in the more than one chamber are connected in line.

72. The device of claim 71 wherein the electroactive polymer pump has a single output port.

73. The device of claim 63, the electroactive polymer pump further comprising:
an inlet port;
an outlet port; and
a check valve between adjacent chambers of the more than one chamber.

74. The device of claim 61 the electroactive polymer pump comprising a plurality of chambers arranged in a planar array having more than one horizontal row, and a plurality of fluid channels connecting the plurality of chambers wherein at least one chamber is connected via a fluid channel to another chamber in a different horizontal row.

75. The device of claim 73 having a single port.

76. The device of claim 71, 72 or 73 arranged into an array.

77. The device of claim 76 wherein the chambers are fluidly coupled vertically.

78. The device of claim 76 wherein the chambers are fluidly coupled horizontally.

79. The device of claim 60 wherein the electroactive polymer pump is a rolled electroactive polymer pump.

80. The device of claim 79 the rolled electroactive polymer pump defining an interior volume in communication with the fluid wherein activation of the rolled electroactive polymer pump forces the fluid into the cavity.

81. The device of claim 79 wherein the rolled electroactive polymer pump is coupled to a drive member so that activation of the rolled electroactive polymer pump moves the drive member wherein movement of the drive member forces the fluid into the cavity.

82. The device of claim 81 wherein the rolled electroactive polymer is a multiple stage electroactive polymer.

83. The device of claim 60 wherein the electroactive polymer pump utilizes efficient polymer actuation configurations.

84. The device of claim 83 herein activation of the electroactive polymer pump utilizing efficient polymer actuation configurations drives a piston that forces fluid into the cavity.

85. The device of claim 60 further comprising a controller configured to receive a signal associated with the cardiac cycle of a heart and generate an actuation signal for the electroactive polymer pump in response thereto.

86. A method for augmenting flow in a body lumen comprising:
detecting a cardiac cycle trigger;
pumping a fluid through the actuation of an electroactive polymer;
deforming at least a portion of a body lumen in response to the cardiac cycle using the pumped fluid.

87. A method for augmenting flow in a body lumen according to claim 86 wherein deforming a portion of a body lumen is performed by porting the pumped fluid into a deformable cuff to deform at least a portion of a body lumen.

88. A method for augmenting flow in a body lumen according to claim 86 wherein the cardiac trigger is related to an ECG of a human.

89. A method for augmenting flow in a body lumen according to claim 86 wherein the first cardiac trigger is related to the increasing portion of the R-wave.

90. A method for augmenting flow in a body lumen according to claim 89 wherein the first cardiac trigger occurs at 90% of the increasing R-wave amplitude.

91. A method for augmenting flow in a body lumen according to claim 86 wherein the actuation of the electroactive polymer causes the deforming at least a portion of a body lumen to coincide with the ventricular systole.

92. A method for augmenting flow in a body lumen according to claim 86 wherein the cardiac cycle trigger is related to aortic pressure.

93. A method for augmenting flow in a body lumen according to claim 86 wherein the actuation of the electroactive polymer causes the deforming at least a portion of a body lumen to augment flow in a copulsation mode.

94. A method for augmenting flow in a body lumen according to claim 86 wherein the deforming at least a portion of a body lumen occurs during the ventricular systole of the heart.

95. A method for augmenting flow in a body lumen according to claim 86 wherein the cardiac trigger is related to the Q-T interval.

96. A method for augmenting flow in a body lumen according to claim 86 wherein the first cardiac trigger is related to the decreasing portion of the T-wave.

97. A method for augmenting flow in a body lumen according to claim 86 wherein the first cardiac trigger occurs at the end of the T-wave.

98. A method for augmenting flow in a body lumen according to claim 86 wherein the cardiac trigger is related to the T-wave and selected so that deformation of at least a portion of a body lumen coincides with the ventricular diastole.

99. A method for augmenting flow in a body lumen according to claim 86 wherein the body lumen is a blood vessel selected from the group consisting of: the ascending aorta, the descending aorta, a set of intercostal arteries, a set of intercostal veins, the superior vena cava, the inferior vena cava, the pulmonary vein and the pulmonary artery.

100. A method for augmenting blood flow in a vessel comprising:
enlarging a cavity formed between a first layer and a second layer by activating an electroactive polymer;
deforming the first layer in response to enlarging the cavity; and
deforming the walls of a vessel adjacent the first layer in response to the deforming of the first layer.

101. A method for augmenting blood flow in a vessel according to claim 100 wherein the deforming the walls of a vessel adjacent the first layer coincides with a portion of a cardiac cycle.

102. A method for augmenting blood flow in a vessel according to claim 100 wherein increasing the pressure in the cavity results in deforming the first layer so as to constrict the vessel.

103. A method for augmenting blood flow in a vessel according to claim 100 wherein increasing the pressure in the cavity constricts the vessel.

104. A method for augmenting blood flow in a vessel according to claim 100 wherein activating an electroactive polymer coincides with a portion of the cardiac cycle.

105. A method for augmenting blood flow in a vessel according to claim 100 wherein deforming the walls of a vessel adjacent the first layer is related to the ECG of the patient.

106. A method for augmenting blood flow in a vessel according to claim 100 wherein deforming the walls of a vessel adjacent the first layer is related to the increasing portion of the R-wave.

107. A method for augmenting blood flow in a vessel according to claim 100 wherein deforming the walls of a vessel adjacent the first layer coincides with the ventricular systole.

108. A method for augmenting blood flow in a vessel according to claim 100 wherein deforming the walls of a vessel adjacent the first layer is related to a change in aortic pressure.

109. A method for augmenting blood flow in a vessel according to claim 100 wherein deforming the walls of a vessel adjacent the first layer is selected such that the blood flow in the vessel is augmented in a copulsation mode.

110. A method for augmenting blood flow in a vessel according to claim 100 wherein activating the electroactive polymer occurs so that the cavity is enlarging during the ventricular systole of the heart.

111. A method for augmenting blood flow in a vessel according to claim 100 further comprising the activating the electroactive polymer is response to a signal associated with a cardiac signal.

112. A method for augmenting blood flow in a vessel according to claim 111 wherein the signal associated with the cardiac cycle is related to the Q-T interval.

113. A method for augmenting blood flow in a vessel according to claim 111 wherein the signal associated with the cardiac cycle is related to the decreasing portion of the T-wave.

114. A method for augmenting blood flow in a vessel according to claim 111 wherein the signal associated with the cardiac cycle occurs at the end of the T-wave.

115. A method for augmenting blood flow in a vessel according to claim 111 wherein the signal associated with the cardiac cycle is related to the T-wave and selected so deforming the walls of a vessel adjacent the first layer coincides with the ventricular diastole.

116. A method for augmenting blood flow in a vessel according to claim 111 wherein the signal is selected such that the blood flow in the vessel is augmented in a counterpulsation mode.

117. A system for compressing a lumen in a body, comprising:
a cuff having a compliant layer and a semi-compliant layer coupled to the compliant layer to form a cavity there between;
an electroactive polymer diaphragm pump having an output; and
a conduit connecting the output and the cavity, wherein activation of the electroactive polymer diaphragm pump expands the compliant layer.

118. The system according to claim 117 wherein the cuff is sufficiently long to completely encircle a lumen selected from the group consisting of: the ascending aorta, the descending aorta, a set of intercostals arteries, a set of intercostals veins, the superior vena cava, the inferior vena cava, the pulmonary vein and the pulmonary artery.

119. The system according the claim 117 wherein the compliant layer is fabricated with a first material and the semi-compliant layer is fabricated with a second material.

120. The system of claim 119, wherein the first material is a first silicone elastomer and the second material is a second silicone elastomer.

121. The system of claim 120, wherein the first silicone elastomer is a 5-50 A silicone elastomer having a minimum of 500% elongation.

122. The system of claim 120, wherein the second silicone elastomer is a 65-95 A silicone elastomer having less than a 400% elongation.

123. The system according to claim 117 wherein the electroactive polymer pump is a single chamber pump.

124. The system according to claim 117 wherein the electroactive polymer pump is a multi-chamber pump.

125. The system according to claim 123 or 124 wherein the electroactive polymer pump has a negative bias.

126. The system according to claims 123 or 124 wherein the electroactive polymer pump has a positive bias.

127. The system according to claim 117 further comprising a sensor for detecting a cardiac signal and a controller for activating the electroactive polymer pump in response to the cardiac signal.

128. A device for compressing a lumen in a body comprising:
a cuff having a compliant layer and a semi-compliant layer and a cavity formed between the compliant layer and the semi-compliant layer;
a deformable fluid reservoir containing a fluid;
a conduit coupling the fluid reservoir to the cavity; and
an electroactive polymer layer including a first electrode, a second electrode and a polymer layer disposed between the first electrode and the second electrode, wherein activation of the electroactive polymer layer deforms the deformable fluid reservoir to urge the fluid into the cavity.

129. The device of claim 128 wherein the activation of the electroactive polymer layer urges fluid into the cavity with sufficient force to deform the compliant layer.

130. The device of claim 128 wherein the electroactive polymer layer partially encircles the deformable fluid reservoir.

131. The device of claim 128 wherein the electroactive polymer layer and the deformable fluid reservoir have the same shape.

132. The device of claim 131 wherein the electroactive polymer layer substantially encompasses the deformable fluid reservoir.

133. The device of claim 128 wherein the shape is spherical.

134. A system, comprising:
an electroactive polymer pump;
controller configured to receive a signal associated with the cardiac cycle of a heart and actuate the electroactive polymer pump in response thereto;
a cuff comprising,
a compliant first layer configured to engage internal vasculature; and
a second layer coupled to the first layer and having a stiffness greater than a stiffness of the first layer and having an opening formed therein;
the compliant first layer and the second layer being coupled to form a cavity bounded by the first layer and the second layer, the cavity being in communication with the opening in the second layer; and
a conduit coupled between the opening and the electroactive polymer pump, wherein actuation of the electroactive polymer pump moves a fluid into the cavity and deforms the first layer.

135. The system of claim 134 wherein the signal associated with the cardiac cycle is related to systole.

136. The system of claim 134 wherein the signal associated with the cardiac cycle is related to diastole.

137. The system of claim 134 wherein the signal associated with the cardiac cycle is related to a change in aortic pressure.

138. The system of claim 134 wherein the signal associated with the cardiac cycle is related to a change in arterial pressure.

139. The system of claim 134 wherein the signal associated with the cardiac cycle is related to a change in venous pressure.

140. The system of claim 134 wherein the electroactive polymer pump is a dielectric electostrictive electroactive polymer pump or an ion-exchange polymer metal electroactive polymer pump.

141. The system of claim 134 wherein the electroactive polymer pump is a rolled electroactive polymer pump.

142. The system of claim 134 wherein the electroactive polymer pump is a diaphragm pump.

143. The system of claim 134 wherein the electroactive polymer pump is a multi-chamber diaphragm pump.

144. The device according to claim 134, the electroactive polymer pump comprising an anode and a cathode wherein the anode and cathode conductivity is about 750 ohms to 1 mega-ohm.

145. The device according to claim 134, the electroactive polymer pump comprising an anode and a cathode wherein an elastomer material separating an anode surface from a cathode surface has a dielectric strength of about 1 kV to 10 kV per mil.

146. The device according to claim 134, the electroactive polymer pump comprising an anode and a cathode wherein an elastomer material separating an anode surface from a cathode surface has a hardness of about 3 A to 75 A durometer.

147. The device according to claim 134, the electroactive polymer pump comprising an anode and a cathode wherein an elastomer material separating an anode surface from a cathode surface has a tensile strength of about 2 to 75 MPa.

148. A system for compressing a blood vessel, comprising:
a cuff having an expandable layer and a cover layer, the cover layer coupled to the expandable layer defining a cavity there between; and
a rolled electroactive polymer pump configured to move a fluid into the cavity to expand the expandable layer in synchronization with a portion of a cardiac cycle.

149. The system according to claim 148 further comprising a sensor for sensing a signal related to the cardiac cycle and a controller wherein the controller actuates the roller electroactive polymer pump in response to the signal related to the cardiac cycle.

150. The system according to claim 149 wherein the cuff, the rolled electroactive polymer pump, the sensor and the controller are all implantable within a body.

151. The system according to claim 149 wherein the controller actuation results in copulsation of a portion of the cardiac cycle.

152. The system according to claim 149 wherein the controller actuation results in counterpulsation of a portion of the cardiac cycle.

153. The system of claim 148, the cover layer further comprising a first end and a second end the ends having a pair of mating fasteners selected from the group consisting of: the mating fasteners are magnets; the mating fasteners have one mating fastener that is a magnet and the other mating fastener is formed from a magnetically attractive material; the mating fasteners are opposite sides of a buckle; the mating fasteners are a screw and a screw-receiving opening; the mating fasteners are a hook and a loop; the mating fasteners comprise a plurality of hooks and a plurality of loops; the mating fasteners include a locking ring and a mating element; and the mating fasteners include a positive-lock set.

154. The system according to claim 148 wherein the cuff is sized to partially encircle a blood vessel selected from the group consisting of: the ascending aorta, the descending aorta, a set of intercostal arteries, a set of intercostal veins, the superior vena cava, the inferior vena cava, the pulmonary vein and the pulmonary artery.l

155. The system according to claim 148 wherein the cuff is sized to completely encircle a blood vessel selected from the group consisting of: the ascending aorta, the descending aorta, a set of intercostal arteries, a set of intercostal veins, the superior vena cava, the inferior vena cava, the pulmonary vein and the pulmonary artery.

156. The system according to claim 148 arranged within a human body such that expansion of the expandable layer compresses a portion of a blood vessel, the blood vessel selected from the group consisting of: the ascending aorta, the descending aorta, a set of intercostal arteries, a set of intercostal veins, the superior vena cava, the inferior vena cava, the pulmonary vein and the pulmonary artery.

157. The system according to claim 148 further comprising a shaft coupled to the rolled electroactive polymer pump wherein actuation of the rolled electroactive polymer pump deflects the shaft.

158. The system according to claim 157 wherein the deflection of the shaft drives a piston to move fluid into the cavity.

159. The system according to claim 148 further comprising a cavity formed within the rolled electroactive polymer pump in communication with the cavity defined by the first layer and the second layer.

160. The system according to claim 159 wherein actuation of the rolled electroactive polymer pump compresses the cavity within the rolled electroactive polymer pump and moves fluid into the cavity defined by the first layer and the second layer.

161. A system for compressing a blood vessel, comprising:
a pair of lever arms coupled at a pivot point; and
a rolled electroactive polymer coupled to an output shaft wherein actuation of the rolled electroactive polymer moves the output shaft; and wherein one of the lever arms is attached to the output shaft.

162. The system according to claim 161 wherein actuation of the rolled electroactive polymer causes a portion of the lever arms to move apart.

163. The system according to claim 161 wherein actuation of the rolled electroactive polymer causes a portion of the lever arms to move together.

164. The system according to claim 161 wherein the lever arms are sized to compress a blood vessel selected from the group consisting of: the ascending aorta, the descending aorta, a set of intercostal arteries, a set of intercostal veins, the superior vena cava, the inferior vena cava, the pulmonary vein and the pulmonary artery.

165. The system according to claim 161 wherein when the lever arm partially encircles a blood vessel actuation of the rolled electroactive polymer compresses the blood vessel between the lever arms.

166. The system according to claim 161 wherein when the lever arms are disposed about a blood vessel the blood vessel is positioned between the pivot point and the output shaft.

167. The system according to claim 161 wherein when the lever arms are disposed about a blood vessel the pivot point is positioned between blood vessel and the output shaft.

168. The system according to claim 161 further comprising a first pair of lever arms coupled at a first pivot point; and a first rolled electroactive polymer coupled to a first output shaft wherein actuation of the first rolled electroactive polymer moves the first output shaft; and wherein one of the lever arms in the first pair of lever arms is attached to the first output shaft and a second pair of lever arms coupled at a second pivot point; and a second rolled electroactive polymer coupled to a second output shaft wherein actuation of the second rolled electroactive polymer moves the second output shaft; and wherein one of the lever arms in the second pair of lever arms is attached to the second output shaft, wherein the pair of lever arms, the first pair of lever arms and the third pair of lever arms are disposed about a blood vessel such that actuation of the rolled electroactive polymer, the second rolled electroactive polymer and the second rolled electroactive polymer compresses the blood vessel.

169. The system according to claim 168 wherein the rolled electroactive polymer, the second electroactive polymer and the third electroactive polymer are actuated simultaneously to compress a blood vessel.

170. The system according to claim 168 wherein the rolled electroactive polymer, the second electroactive polymer and the third electroactive polymer are actuated sequentially to compress a blood vessel.

171. A device for compressing a blood vessel, comprising:
a first layer comprising an electroactive polymer and coupled to a second layer;
the second layer having a length sufficient to at least partially encircle a body lumen and a stiffness greater than that of the first layer;
a cavity formed between the first layer and the second layer; and
a bias element disposed within the cavity and configured to expand the electroactive polymer when the electroactive polymer is in an non-actuated state.

172. The device according to claim 171 wherein the bias element is a foam material.

173. The device according to claim 171 wherein the bias element is a spring.

174. The device according to claim 171 wherein the bias element comprises a fluid.

175. A device for compressing a blood vessel in a body, comprising:
a deformable bladder containing a fluid;
a cuff having an expandable layer and a cover layer, the cover layer coupled to the expandable layer to define a cavity there between; and
a “C” ring electroactive polymer actuator disposed about the bladder such that actuation of the electroactive polymer actuator deforms the bladder and forces fluid into the cavity.

176. A device for compressing a blood vessel in a body according to claim 175 further comprising a plurality of “C” ring electroactive polymer actuators.

177. A device for compressing a blood vessel in a body according to claim 176 wherein the plurality of “C” ring electroactive polymer actuators are actuated serially.

178. A device for compressing a blood vessel in a body according to claim 176 wherein the plurality of “C” ring electroactive polymer actuators are actuated simultaneously.

179. A device for compressing a blood vessel in a body according to claim 176 wherein the plurality of “C” ring electroactive polymer actuators are actuated sequentially.

180. A device for compressing a blood vessel in a body according to claim 175 wherein the “C” ring electroactive polymer actuator is actuated in response to a cardiac signal.

181. A device for compressing a blood vessel in a body according to claim 175 wherein the “C” ring electroactive polymer actuator is actuated in response to a signal.

182. A method for augmenting blood flow in a body, comprising:
sensing the R wave of the ECG of the body;
computing the QT interval to the end of the T wave; and
actuating an electroactive polymer based vascular assist system in relation to the T wave.

183. A method for augmenting blood flow in a body according to claim 182 wherein the actuation of an electroactive polymer system augments blood flow in a counterpulsation mode.

184. A method for augmenting blood flow in a body according to claim 182 wherein the actuation of the electroactive polymer system augments blood flow during diastole.

185. A method for augmenting blood flow in a body according to claim 182 wherein the actuation of an electroactive polymer system augments blood flow in a co-pulsation mode.

186. A method for augmenting blood flow in a body according to claim 182 wherein the actuation of the electroactive polymer system augments blood flow during systole.

187. A method for augmenting blood flow in a body according to claim 182 wherein actuating the electroactive polymer based system augments blood flow by using electroactive polymer actuation to pump a fluid into an expanding wall cuff disposed about a body lumen.

188. A method for augmenting blood flow in a body according to claim 182 wherein actuating the electroactive polymer based system augments blood flow by using electroactive polymer actuation to compress a body lumen.

189. A method for augmenting blood flow in a body according to claim 182 wherein actuating the electroactive polymer based system augments blood flow by using electroactive polymer actuation to compress a deformable bladder.

190. A method for augmenting blood flow in a body, comprising:
sensing a pressure wave related to a hemodynamic pressure in the body; and
based on a portion of the pressure wave, actuating an electroactive polymer based system to augment blood flow in the body.

191. A method for augmenting blood flow in a body according to claim 190 wherein the pressure in the body is venous pressure.

192. A method for augmenting blood flow in a body according to claim 190 wherein the pressure in the body is arterial pressure.

193. A method for augmenting blood flow in a body according to claim 190 wherein actuating the electroactive polymer based system augments blood flow by using electroactive polymer actuation to pump a fluid into an expanding wall cuff disposed about a body lumen.

194. A method for augmenting blood flow in a body according to claim 190 wherein actuating the electroactive polymer based system augments blood flow by using electroactive polymer actuation to compress a body lumen.

195. A method for augmenting blood flow in a body according to claim 190 wherein actuating the electroactive polymer based system augments blood flow by using electroactive polymer actuation to compress a deformable bladder.

196. A method of forming a stacked electroactive polymer actuator, comprising:
forming a plurality of adjacent electrodes on a single polymer layer; and
folding the polymer layer so that adjacent electrodes are stacked so that at least a single polymer layer exists between each adjacent electrode.

197. A system for augmenting blood flow, comprising:
a conventional vascular assist system selected from the group consisting of: an impeller driven left ventricle assist device, a solenoid driven vascular assist device and a total artificial heart, the conventional vascular assist system being modified to include an electroactive pump as the motive force for the movement of blood through the vascular assist device.
